(19) United States
(12) Patent Application Publication
(10) Pub. No.: US 2009/0069302 A1
(43) Pub. Date: Mar. 12, 2009

(54) CHEMICAL COMPOUNDS

(75) Inventors: Jerzy Ryszard Szewczyk, Durham, NC (US); Christopher P. Laudeman, Durham, NC (US); Thomas Dock, Durham, NC (US); Zibin Chen, Durham, NC (US); Anderson Evans, Collegeville, PA (US); Yue H. Li, Collegeville, PA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadephia, PA (US)

(21) Appl. No.: 12/160,792

(22) PCT Filed: Jan. 19, 2007
(86) PCT No.: PCT/US2007/060759

§ 371 Date: Jul. 14, 2008

Related U.S. Application Data
(60) Provisional application No. 60/760,840, filed on Jan. 20, 2006.

(51) Int. Cl. A61K 31/551; C07D 241/04; A61K 31/495; A61K 31/18; C07C 311/16; C07D 243/08

(52) U.S. Cl. 514/218; 544/383; 514/252.12; 540/575; 564/85; 514/603

(57) ABSTRACT

The present invention relates generally to novel therapeutic compounds and AXOR 109 agonists, and processes for the manufacture and use of the same.

### FIELD OF THE INVENTION

The present invention relates generally to novel therapeutic compounds and AXOR 109 agonists, and processes for the manufacture and use of the same.

### BACKGROUND OF THE INVENTION

Diabetes mellitus is an ever-increasing threat to human health. For example, in the United States current estimates maintain that about 16 million people suffer from diabetes mellitus.

Type II diabetes accounts for approximately 90-95% of diabetes cases, killing about 193,000 U.S. residents each year. Type II diabetes is the seventh leading cause of all deaths. In Western societies, type II diabetes currently affects 6% of the adult population with world-wide frequency expected to grow by 6% per annum. Although there are certain inheritable traits that may predispose particular individuals to developing type II diabetes, the driving force behind the current increase in incidence of the disease is the increased sedentary life-style, diet, and obesity now prevalent in developed countries. About 80% of diabetics with type II diabetes are significantly overweight. Also, an increasing number of young people are developing the disease. Type II diabetes is now internationally recognized as one of the major threats to human health in the 21^{st} century.

Type II diabetes, also known as non-insulin-dependent diabetes mellitus, manifests as an inability to adequately regulate blood-glucose levels. Type II diabetes may be characterized by a defect in insulin secretion or by insulin resistance. Namely, those that suffer from Type II diabetes have too little insulin or cannot use insulin effectively. Insulin resistance refers to the inability of the body tissues to respond properly to endogenous insulin. Insulin resistance develops because of multiple factors, including genetics, obesity, increasing age, and having high blood sugar over long periods of time. Type II diabetes, sometimes called mature onset, can develop at any age, but most commonly becomes apparent during adulthood. However, the incidence of type II diabetes in children is rising.

In diabetics glucose levels build up in the blood and urine causing excessive urination, thirst, hunger, and problems with fat and protein metabolism. If left untreated, diabetes mellitus may cause life-threatening complications, including blindness, kidney failure, and heart disease.

Type II diabetes is currently treated at several levels. A first level of therapy is through diet and/or exercise, either alone or in combination with therapeutic agents. Such agents may include insulin or pharmaceuticals that lower blood glucose levels. About 49% of individuals with Type II diabetes require oral medications, about 40% require insulin injections or a combination of insulin injections and oral medications, and 10% use diet and exercise alone.

Current therapies include: insulin secretagogues, such as sulphonylureas, which increase insulin production from pancreatic β-cells; glucose-lowering effectors, such as mefformin which reduce glucose production from the liver; activators of the peroxisome proliferator-activated receptor-γ (PPAR-γ), such as the thiazolidinediones, which enhances insulin action; and α-glucosidase inhibitors which interfere with gut glucose production. There are, however, deficiencies associated with currently available treatments. For example sulphonylureas and insulin injections can be associated with hypoglycemic episodes and weight gain. Furthermore, patients often lose responsiveness to sulphonylureas over time. Metformin and α-glucosidase inhibitors often lead to gastrointestinal problems and PPAR-γ agonists tend to cause increased weight gain and edema.

AXOR 109, also known as TGR5, BG37, M-BAR, or hGPCR19, is a bile acid G-protein coupled receptor primarily expressed in monocytes and macrophages, lung, spleen, and the intestinal tract. In response to bile acids, AXOR 109 has been shown to cause a dose dependent elevation in intracellular concentrations of cAMP in cells that express the receptor. Maruyama, T et al., Biochem. Biophys. Res. Comm. 298 (2002) 714-719. Bile acids and compounds that affect AXOR 109 have also been shown to increase glucagon like peptide-1 (GLP-1) secretion from primary intestinal cells. It has been suggested that bile acids induce GLP-1 secretion by increasing intracellular cAMP levels via through AXOR 109. Katsuma, S. et al., Biochem Biophys Res Commun. 2005 Apr. 1; 329(1):386-90.

GLP-1 is a peptide secreted from enteroendocrine L cells, and has a wide variety of physiological effects that have been described in numerous publications over the past two decades. More recently, much research has been focused on the use of GLP-1 in the treatment of conditions and disorders such as diabetes mellitus, stress, obesity, appetite control and satiety, alzheimers, inflammation, and diseases of the central nervous system. See, for example, Bojanowska, E. et al., Med. Sci. Monit., 2005, Aug. 11 (8): RA271-8; Perry, T. et al., Curr. Alzheimer Res., 2005, Jul. 2(3): 377-85; and Meier, J. J. et al., Diabetes Metab. Res. Rev., 2005, March-April; 21(2); 91-117. However, the use of a peptide in clinical treatment is severely limited due to difficult administration, and in vivo stability. Therefore, a small molecule that either mimicked the effects of GLP-1 directly, or increased GLP-1 secretion, may be useful in treatment of the variety of conditions or disorders described above, namely diabetes mellitus.

### SUMMARY OF THE INVENTION

The present invention identifies agonists of AXOR 109, which may be useful in treating a variety of conditions and disorders shown to be affected by GLP-1 activity.

One aspect of the present invention is a pharmaceutical composition comprising a compound selected from
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1,4-bis{[3,4-bis(methyloxy)phenyl]sulfonyl}piperazine;
1-{[3,4-bis(methyloxy)phenyl]sulfonyl}-4-[(2-chloro-6-methyl phenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
1,4-bis{[3,4-bis(methyloxy)phenyl]sulfonyl}hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-bis(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1,3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutyl methyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;

or a salt or solvate thereof.

Another aspect of the present invention is the administration of a pharmaceutical composition of the present invention in a method for the treatment of conditions or disorders that are affected by AXOR109.

Another aspect of the present invention is the administration of a pharmaceutical composition of the present invention in a method for the treatment of conditions or disorders that are affected by GLP-1.

Another aspect of the present invention includes the use of a compound selected from:
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1,4-bis{[3,4-bis(methyloxy)phenyl]sulfonyl}piperazine;
1-{[3,4-bis(methyloxy)phenyl]sulfonyl}-4-[(2-chloro-6-methylphenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
1,4-bis{[3,4-bis(methyloxy)phenyl]sulfonyl}hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-bis(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1,3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutyl methyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;

or a salt or solvate thereof, in the manufacture of a medicament for use in the treatment of conditions or disorders that are affected by AXOR109.

Another aspect of the present invention includes the use of a compound selected from:
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1,4-bis{[3,4-bis(methyloxy)phenyl]sulfonyl}piperazine;
1-{[3,4-bis(methyloxy)phenyl]sulfonyl}-4-[(2-chloro-6-methylphenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
1,4-bis{[3,4-bis(methyloxy)phenyl]sulfonyl}hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-bis(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1,3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutylmethyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;

or a salt or solvate thereof, in the manufacture of a medicament for use in the treatment of conditions or disorders that are affected by GLP-1.

Another aspect of the present invention includes a compound for use in therapy selected from:
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1,4-bis{[3,4-bis(methyloxy)phenyl]sulfonyl}piperazine;
1-{[3,4-bis(methyloxy)phenyl]sulfonyl}-4-[(2-chloro-6-methylphenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
1,4-bis{[3,4-bis(methyloxy)phenyl]sulfonyl}hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N1,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-bis(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1,3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutyl methyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;
   or a salt or solvate thereof.

Another aspect of the present invention includes a compound selected from:
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1-{[3,4-bis(methyloxy)phenyl]sulfonyl}-4-[(2-chloro-6-methylphenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-bis(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1,3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutylmethyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;
   or a salt or solvate thereof.

Another aspect of the present invention is the administration of a compound of the present invention in a method for the treatment of conditions or disorders that are affected by AXOR109.

Another aspect of the present invention is the administration of a compound of the present invention in a method for the treatment of conditions or disorders that are affected by GLP-1.

### BRIEF DESCRIPTION OF THE DRAWINGS

FIGS. 1a and 1b demonstrate the effect of a representative compound of the present invention on GLP-1 secretion in CD rats. FIGS. 1a and 1b are the graphical representations of the data contained in Tables 2a and 2b, respectively.

FIGS. 2a, 2b, and 2c demonstrate the effect of a representative compound of the present invention on glucose, insulin, and GLP-1 secretion in GK rats. FIGS. 2a, 2b, and 2c are the graphical representations of the data contained in Tables 3a, 3b, and 3c, respectively.

FIGS. 3a and 3b demonstrate the effect of a representative compound of the present invention on the prevention of hyperglycemia, increased insulin levels, and increased glucagon levels after chronic dosing in GK rats. FIGS. 3a and 3b are the graphical representations of the data contained in Tables 4a and 4b, respectively.

FIGS. 4a, 4b, 4c, and 4d demonstrate the effect of a representative compound of the present invention on glucose tolerance in GK rats. FIGS. 4a, 4b, 4c, and 4d are the graphical representations of the data contained in Tables 5a, 5b, 5c, and 5d, respectively.

### DETAILED DESCRIPTION OF THE PREFERRED EMBODIMENT

The present invention is described in terms known and appreciated by those skilled in the art. For ease of reference certain terms hereinafter are defined. The fact that certain terms are defined, however, should not be considered as indicative that defined terms are used in a manner necessarily inconsistent with the ordinary meaning or, alternatively, that any term that is undefined is indefinite or not used within the ordinary and accepted meaning. Rather, all terms used herein are believed to describe the invention such that one of ordinary skill can appreciate the scope of the present invention.

As used herein, “a compound of the present invention” includes all compounds described in the Examples herein.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may include acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of the present invention or a salt thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

For use in therapy, therapeutically effective amounts of a compound of the present invention, as well as salts and solvates thereof, may be administered as the raw chemical. Additionally, the active ingredient may be presented as a pharmaceutical composition.

As used herein, the term “treatment” refers to alleviating the specified condition, eliminating or reducing the symptoms of the condition, slowing or eliminating the progression of the condition and preventing or delaying the initial occurrence of the condition in a subject, or reoccurrance of the condition in a previously afflicted subject.

The present invention provides pharmaceutical compositions that include effective amounts of a compound as herein described, or a salt or solvate thereof, and one or more pharmaceutically acceptable carriers, diluents, or excipients. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation, including admixing a compound of the present invention or salts or solvates thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from diabetes and associated conditions, generally, should be in the range of 0.01 to 100 mg/kg body weight of recipient (mammal) per day. More usually the effective amount should be in the range of 0.1 to 10 mg/kg body weight per day. Thus, for a 70 kg adult mammal the actual amount per day would usually be from 7 to 700 mg. This amount may be given in a single dose per day or in a number (such as two, three, four, five, or more) of sub-doses per day such that the total daily dose is the same. An effective amount of a salt or solvate thereof, may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the present invention, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful, certain routes will be preferable over others. For example, oral administration is preferred for many diabetic therapy regimens.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules can be made by preparing a powder, liquid, or suspension mixture and then encapsulating such with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets can be formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle.

Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the present invention or a salt or solvate thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of the present invention or a salt or solvate thereof may also be delivered by the use of monoclonal antibodies as individual carriers to which the compound molecules are coupled.

In one embodiment of the present invention, compounds of the present invention, or a salt or solvate thereof, are administered by a targeted drug delivery system. Preferably, the delivery systems may be employed for targeting drug delivery to the lower gastrointestinal tract or colon. Such drug delivery systems include covalent linkage compositions, polymer coated compositions, compositions embedded in matrices, time-released compositions, redox-sensitive polymer compositions, bioadhesive compositions, micropartical coating compositions, and osmotic delivery compositions. See, for example, Chourasia, M. K. et al. J Pharm Pharmaceut. Sci., 6(1):33-66, 2003, and references therein, herein incorporated by reference to the extent that they teach the employment of targeted drug delivery systems. Suitable compositions include those containing polysaccharides such as chitosan, pectin, chondroitin sulphate, cyclodextrin, dextrans, guar gum, inulin, amylose and locust bean gum. See, for example, Sinha, V. R. et al., International Journal of Pharmaceutics, 224, (2001) 19-38. The compounds may also be coupled with soluble polymers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. Those skilled in the art will appreciate the use of such compositions for the purposes of targeting delivery of the compounds of the present invention, or a salt or solvate thereof, to the lower gastrointestinal tract of the subject being treated.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention or a salt or solvate thereof, and the pharmaceutical compositions of the present invention, may be useful for treating conditions or disorders affected by AXOR 109 and/or GLP-1, and include, but are not limited to, type I diabetes, type II diabetes, obesity, appetite control, satiety, glucose intolerance, insulin resistance, metabolic syndrome, hyperlipidemia, hypercholesterolemia, atherosclerosis, inflammation, neurodegenerative diseases, Alzheimer's, stress disorders, and cerebrovascular conditions. Preferably, the conditions or disorders are type I diabetes, type II diabetes, glucose intolerance, insulin resistance, and metabolic syndrome.

The compounds of the present invention or a salt or solvate thereof, may be employed alone or in combination with other therapeutic agents. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention or a salt or solvate thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions. As such, the compounds of the present invention may be used in combination with a variety of other therapeutic agents useful in the treatment of those disorders or conditions. As discussed briefly above, current diabetes therapies include diet, exercise, insulin, insulin secretagogues, glucose-lowering effectors, PPAR-γ agonists, and α-glucosidase inhibitors. The compounds of the present invention may be combined with these or other medical therapies to treat and/or prevent diabetes and associated disorders and conditions, including but not limited to diabetes types I and II, obesity, glucose intolerance, insulin resistance, metabolic syndrome, hyperlipidemia, hypercholesterolemia, artheroscelrosis, neurodegenerative diseases, and other indications such as inflammation and stroke. For example, in the treatment of type II diabetes, a compound of the present invention may be combined with one or more pharmaceutically active agents, including metformin, sulfonylureas such as glyburide and glipizide, repaglinide, nateglinide, thiazolidinediones such as rosiglitazone and pioglitazone, acarbose, miglitol, exanatide, pramlintide, and insulin.

The compounds of this invention may be made by a variety of methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the synthesis using methods that are readily apparent to those skilled in the art. The selection of processes as well as the reaction conditions and order of their execution shall be consistent with the preparation of compounds of the present invention.

Those skilled in the art will recognize if a stereocenter exists in compounds of the present invention. Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
rt (room temperature); min (min);
h (hours); mp (melting point);
TLC (thin layer chromatography); CH₂Cl₂ (methylene chloride);
t_{R} (retention time); RP (reverse phase);
TEA (triethylamine); TFA (trifluoroacetic acid);
TFAA (trifluoroacetic anhydride); THF (tetrahydrofuran);
CDCl₃ (deuterated chloroform); CD₃OD (deuterated methanol);
SiO₂ (silica); DMSO (dimethylsulfoxide);
EtOAc (ethyl acetate); atm (atmosphere);
HCl (hydrochloric acid); CHCl₃ (chloroform);
DMF (N,N-dimethylformamide); Ac (acetyl);
Cs₂CO₃ (cesium carbonate); Me (methyl);
Et (ethyl); EtOH (ethanol);
MeOH (methanol); t-Bu (tertiary-butyl);
PPTS (pyridinium p-toluenesulfonate);
DME (1,2-dimethoxyethane); N₂ (nitrogen).
ADDP (1,1′-(azodicarbonyl)dipiperidine);
NMO (4-methylmorpholine N-oxide); DBAD (dibenzyl azodicarboxylate);
9-BBN (9-borabicyclo[3.3.1]nonyl); CsF (cesium fluoride);
MsCl (methanesulphonyl chloride); sat'd (saturated);
KOAc (potassium acetate); DCC (1,3-dicyclohexylcarbodiimide);
BOC (tert-butoxycarbonyl group); Ac (acetyl group);
Ps (polymer supported); DCM (dichloromethane);
DIEA (diisopropylethylamine); conc. (concentrated);
CBz (benzyloxy carbonyl); TPP (triphenylphosphine);
DIAD (diisopropylazodicarboxylate).

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, a Bruker 400 UltraShield spectrometer, or a General Electric QE-300. Chemical shifts are expressed in parts per million (ppm, δ units). Splitting patterns describe apparent multiplicities and are designated as s (singlet), d (doublet), t (triplet), q (quartet), m (multiplet), or b (broad).

Compounds of the present invention may be prepared by the processes outlined below. The order of the following steps may not be critical to the practice of the invention, and the processes may be practiced by performing the steps in any suitable order based on the knowledge of those skilled in the art. Compounds of the invention can be prepared using Methods A through F described below in which each J₁-J₆ independently represent s various groups including, but not limited to, alkyl, substituted alkyl, branched alkyl, cyclic alkyl, aryl, heteroaryl, or substituted aryl; each Z independently represents various groups including, but not limited to, methylene, carbonyl, alkylated methylene, halogenated methylene, or fused aryl; each Y independently represents nothing (n=0) or an atom such as carbon or oxygen; each X independently represents a halogen such as iodine, bromine, or chlorine; and each n independently is 0 or an integer such as 1, 2, 3, or 4.

Method A (Solution-Phase Synthesis of Compounds of the Present Invention from Intermediate A.)

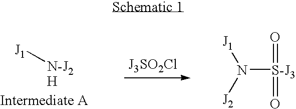

In Method A, Intermediate A is mixed with a sulfonyl chloride, J₃SO₂Cl, in solvent such as methylene chloride with base such as diisopropylethylamine, triethylamine, pyridine, or preferably triethylamine in methylene chloride. The reactions can be heated, but are preferably mixed at room temperature. Intermediate A is prepared by an analogous procedure or according to Lima et al. in Bioorg. Med. Chem. 2002, 10, 3067-73.

Method B (Solution-Phase Synthesis of Compounds of the Present Invention from Diamines.)

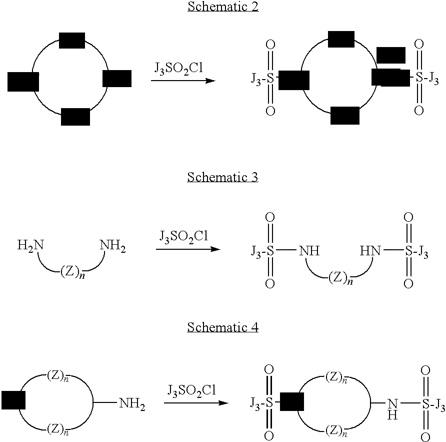

In Method B, a diamine is mixed with at least 2 equivalents of a sulfonyl chloride, J₃SO₂Cl, in solvent such as methylene chloride with base such as diisopropylethylamine, triethylamine, sodium hydride, pyridine, or preferably triethylamine in methylene chloride. The reactions can be heated, but are preferably mixed at room temperature.

Method C (Solution-Phase Synthesis of Compounds of the Present Invention from Diamines.)

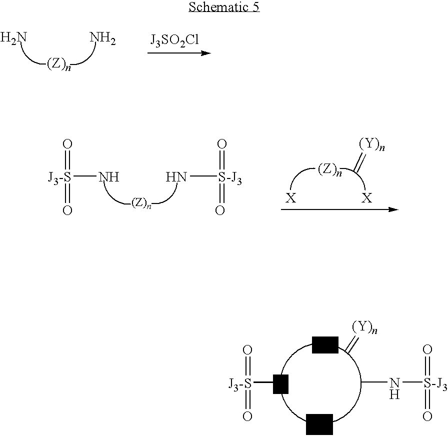

In Method C, the acyclic diamine is mixed with at least 2 equivalents of a sulfonyl chloride, J₃SO₂Cl, in solvent such as methylene chloride with base such as diisopropylethylamine, triethylamine, sodium hydride, pyridine, or preferably triethylamine in methylene chloride to form the bis-sulfonamide product. The reactions can be heated, but are preferably mixed at room temperature. The product is then treated with an appropriate acid halide or alkyl halide such as ethanedioyl dichloride, 3-chloropropanoyl chloride, or 1,3-dibromobutane in solvent such as methylene chloride with base such as diisopropylethylamine, triethylamine, pyridine, or preferably triethylamine in methylene chloride. The reactions can be heated, but are preferably mixed at room temperature.

Method D (Solution-Phase Synthesis of Compounds of the Present Invention from Diamines.)

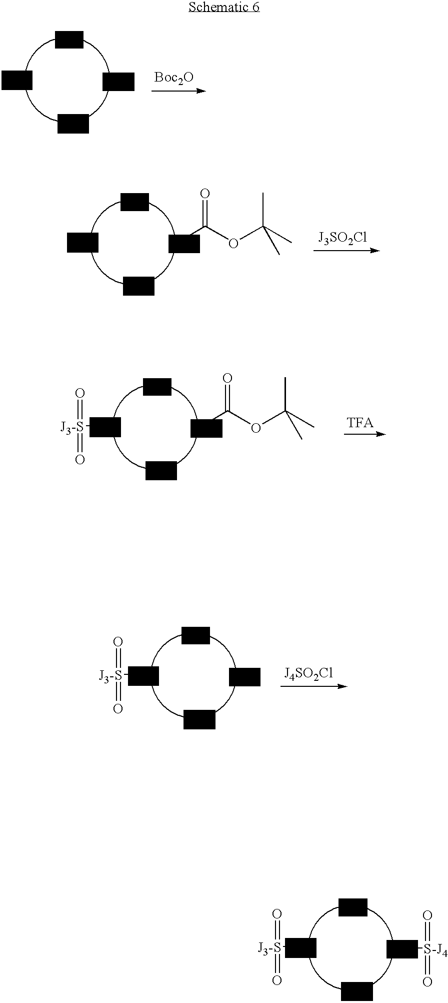

In Method D, the cyclic diamine is mixed with bis(1,1-dimethylethyl)dicarbonate (Boc₂O) in solvent such as methylene chloride to form the Boc monoprotected product. The reactions can be run at room temperature, but are preferably mixed at ° C. The Boc amine is then mixed with at least 1 equivalent of a sulfonyl chloride, J₃SO₂Cl, in solvent such as methylene chloride with base such as diisopropylethylamine, triethylamine, sodium hydride, pyridine, or preferably pyridine and methylene chloride to form the sulfonamide product. The reactions are preferably mixed at 80° C. The Boc group is then removed by treatment with trofluoroacetic acid (TFA) in solvent such as methylene chloride (DCM), preferably 50% TFA in DCM at room temperature. The reactions can be heated, but are preferably mixed at room temperature. Lastly, the amine is then mixed with at least 1 equivalent of a sulfonyl chloride, J₄SO₂Cl, in solvent such as methylene chloride with base such as diisopropylethylamine, triethylamine, sodium hydride, pyridine, or preferably pyridine to form the bis-sulfonamide product. The reactions are preferably mixed at 80° C.

Method E (Solution-Phase Synthesis of Compounds of the Present Invention from N,N′-bissulfonyldiamines.)

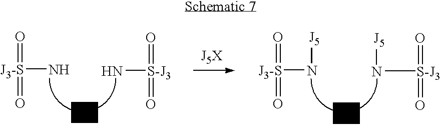

In Method E, an acyclic bis-sulfonamide is mixed with at least 2 equivalents of an alkylating agent, J₅X, in solvent such as acetonitrile, acetone, or DMSO with base such as potassium carbonate, cesium carbonate, sodium hydride or preferably potassium carbonate in acetonitrile to form the alklated bis-sulfonamide product. The reactions can be heated, but are preferably mixed at room temperature.

Method F (Solution-Phase Synthesis of Compounds of the Present Invention from N,N′-bissulfonyldiamines.)

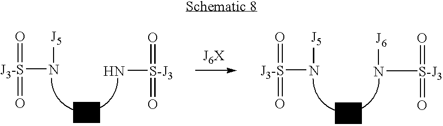

In Method F, an acyclic bis-sulfonamide is mixed with at least 1 equivalent of an alkylating agent, J₆X, in solvent such as acetonitrile, acetone, or DMSO with base such as potassium carbonate, cesium carbonate, sodium hydride or preferably potassium carbonate in acetonitrile to form the alklated bis-sulfonamide product. The reactions can be heated, but are preferably mixed at room temperature.

### EXAMPLES

The following examples are intended for illustration only and are not intended to limit the scope of the invention in any way. Unless otherwise noted, reagents are commercially available or are prepared according to procedures described in the literature. Where not included with the preparations, characterizing data are reported in Table 1 at the end of this section.

Chromatographic purifications of final products were carried out using reverse phase high pressure liquid chromatography, unless otherwise specified. Chromatographic purification of intermediates, when necessary, was carried out using silica gel under air pressure. Reactions were carried out in suitable containers, which can include polypropylene or Teflon tubes or deep well plates, or glass vessels.

### Intermediate Example 1

### 5-(chlorosulfonyl)-2-methoxyphenyl Methanesulfonate

a). Preparation of 2-methoxyphenyl methanesulfonate: To a cooled (0° C.) solution of guaincol (5.05 g, 124.1 mmol) and 25 mL of dichloromethane (DCM) was added triethylamine (8.5 mL, 61.04 mmol) and methanesulfonyl chloride (4.72 mL, 61.04 mmol). After stirring for 2 h the reaction appeared complete by TLC. The reaction solution was diluted with saturated ammonium chloride and water and the organic phase isolated and concentrated to oil in vacuo. The crude material was purified by chromatography on silica gel eluting with ethyl acetate/hexane. The desired product was afforded as a clear oil (7.5 g, 91% yield). ¹H NMR (DMSO-d6, 400 MHz) δ: 7.31 (1H, dq), 7.28 (1H, dd), 7.19 (1H, dd), 6.97 (1H, dq), 3.82 (s, 3H), 3.32 (3H, s).

b). Preparation of 5-(chlorosulfonyl)-2-methoxyphenyl methanesulfonate: To a cooled (−10° C.) solution of chlorosulfonic acid (11.78 mL, 176.5 mmol) and 100 mL of DCM was added dropwise a solution of 2-methoxyphenyl methanesulfonate (7.13 g, 35.3 mmol) dissolved in 15 mL DCM. After 4.5 h the reaction was added to ice, the organic phase isolated, dried over Na2SO4, filtered and concentrated in vacuo to a light yellow oil which solidified to afford product as white solid (10.04 g, 95% yield). ¹H NMR (DMSO-d6, 400 MHz) δ: 7.52 (1H, dd), 7.42 (1H, d), 7.14 (1H, d), 3.83 (3H, s), 3.31 (3H, s).

### Example 1

### 1-{[3,4-bis(methyloxy)phenyl]sulfonyl}-4-{[3-fluoro-4-(methyloxy)phenyl]sulfonyl}piperazine (Method A, Schematic 1)

a). Preparation of 1-{[3,4-bis(methyloxy)phenyl]sulfonyl}piperazine: To a solution of 3,4-bis(methyloxy)benzenesulfonyl chloride (4 g, 16.9 mmol) in 282 mL dichloromethane (DCM), was added piperazine (2.91 g, 2 eq) all at once. The reaction appeared complete by LCMS within minutes. After stirring overnight, the reaction progress was unchanged by LCMS. The mixture was concentrated by half in vacuo. Water was added and the solution acidified by addition of 1N hydrochloric acid (HCl). The organic layer was discarded. The aqueous layer was washed 1× with DCM, and then made basic by addition of 1N sodium hydroxide (NaOH). The aqueous phase was extracted with ethyl acetate (6×40 mL each). The combined organic phases were dried over magnesium sulfate (MgSO₄), filtered, concentrated, and dried in vacuo. The final product was afforded as a white solid and 100% purity by LCMS (M+H=287.2) and NMR (3.19 g, 65% yield). ¹H NMR (DMSO-d6, 400 MHz) δ: 7.29 (1H, dd), 7.19 (1H, d), 7.13 (1H, d), 3.86 (3H, s), 3.83 (3H, s), 2.76 (4H, br q), 2.70 (4H, br q).

b). Preparation of 1-{[3,4-bis(methyloxy)phenyl]sulfonyl}-4-{[3-fluoro-4-(methyloxy)phenyl]sulfonyl}piperazine: In a single well of a Robbins Flex-Chem System 96 well 2.5 mL reaction block was added 1-{[3,4-bis(methyloxy)phenyl]sulfonyl}piperazine (60 umol, 500 uL, 0.12 M solution in DCM). Triethylamine was added (1.5 eq, 12.5 uL), followed by 3-fluoro-4-(methyloxy)benzenesulfonyl chloride (1.1 eq, 500 uL, 0.13 M solution in DCM). The block was sealed and rotated overnight at room temperature. After 15 h, the reaction was filtered and collected by vacuum filtration. The reaction solids were washed with DCM (1×200 uL). The crude reaction mixture was concentrated in vacuo, dissolved in 700 uL DMSO, and purified using reverse-phase HPLC to afford the final product.

### Example 2

### 1-{[3,4-bis(methyloxy)phenyl]sulfonyl}-4-{[4-(1,3-oxazol-5-yl)phenyl]sulfonyl}piperazine

This compound was prepared as described in Example 1, except that 4-(1,3-oxazol-5-yl)benzenesulfonyl chloride was substituted for 3-fluoro-4-(methyloxy)benzenesulfonyl chloride.

### Example 3

### 1-{[3,4-bis(methyloxy)phenyl]sulfonyl}-4-{[4-methyl-2-(methyloxy)phenyl]sulfonyl}piperazine

This compound was prepared as described in Example 1 except that 4-methyl-2-(methyloxy)benzenesulfonyl chloride was substituted for 3-fluoro-4-(methyloxy)benzenesulfonyl chloride.

### Example 4

### 1,4-bis{[3,4-bis(methyloxy)phenyl]sulfonyl}piperazine

This compound was prepared as described in Example 1 except that 3,4-bis(methyloxy)benzenesulfonyl chloride was substituted for 3-fluoro-4-(methyloxy)benzenesulfonyl chloride.

### Example 5

### 1-{[3,4-bis(methyloxy)phenyl]sulfonyl}-4-[(2-chloro-6-methylphenyl)sulfonyl]piperazine

This compound was prepared as described in Example 1 except that 2-chloro-6-methylbenzenesulfonyl chloride was substituted for 3-fluoro-4-(methyloxy)benzenesulfonyl chloride.

### Example 6

### 1-{[3,4-bis(methyloxy)phenyl]sulfonyl}-4-{[4-(methyloxy)phenyl]sulfonyl}piperazine

This compound was prepared as described in Example 1 except that 4-(methyloxy)benzenesulfonyl chloride was substituted for 3-fluoro-4-(methyloxy)benzenesulfonyl chloride.

### Example 7

### 1-{[3,4-bis(methyloxy)phenyl]sulfonyl}-4-{[3-fluoro-4-(methyloxy)phenyl]sulfonyl}hexahydro-1H-1,4-diazepine

a). Preparation of 1-{[3,4-bis(methyloxy)phenyl]sulfonyl}hexahydro-1H-1,4-diazepine: This compound was prepared as described in Example 1a except that homopiperazine was substituted for piperazine. The final product was afforded as a clear, colorless oil and 100% purity by LCMS (M+H=301.4) and NMR (3.67 g, 72% yield). ¹H NMR (DMSO-d6, 400 MHz) δ: 7.35 (1H, dd), 7.20 (1H, d), 7.13 (1H, d), 3.84 (3H, s), 3.83 (3H, s), 3.25 (2H, dd), 3.18 (2H, dd), 2.73 (2H, dd), 2.68 (2H, dd), 1.63 (1H, quintet).

b). Preparation of 1-{[3,4-bis(methyloxy)phenyl]sulfonyl}-4-{[3-fluoro-4-(methyloxy)phenyl]sulfonyl}hexahydro-1H-1,4-diazepine: This compound was prepared as described in Example 1 except that 1-{[3,4-bis(methyloxy)phenyl]sulfonyl}hexahydro-1H-1,4-diazepine, prepared as in Example 7a, was substituted for 1-{[3,4-bis(methyloxy)phenyl]sulfonyl}piperazine.

### Example 8

### 1-{[3,4-bis(methyloxy)phenyl]sulfonyl}-4-(2,3-dihydro-1,4-benzodioxin-6-ylsulfonyl)hexahydro-1H-1,4-diazepine

This compound was prepared as described in Example 1 except that 1-{[3,4-bis(methyloxy)phenyl]sulfonyl}hexahydro-1H-1,4-diazepine, prepared as in Example 7a, was substituted for 1-{[3,4-bis(methyloxy)phenyl]sulfonyl}piperazine, and 2,3-dihydro-1,4-benzodioxin-6-sulfonyl chloride was substituted for 3-fluoro-4-(methyloxy)benzenesulfonyl chloride.

### Example 9

### 1,4-bis{[3,4-bis(methyloxy)phenyl]sulfonyl}hexahydro-1H-1,4-diazepine

This compound was prepared as described in Example 1 except that 1-{[3,4-bis(methyloxy)phenyl]sulfonyl}hexahydro-1H-1,4-diazepine, prepared as in Example 7a, was substituted for 1-{[3,4-bis(methyloxy)phenyl]sulfonyl}piperazine, and 3,4-bis(methyloxy)benzenesulfonyl chloride was substituted for 3-fluoro-4-(methyloxy)benzenesulfonyl chloride.

### Example 10

### N-methyl-N-[2-(methyl{[3-(methyloxy)phenyl]sulfonyl}amino)ethyl]-3,4-bis(methyloxy)benzenesulfonamide

a). Preparation of N-methyl-N-[2-(methylamino)ethyl]-3,4-bis(methyloxy)benzenesulfonamide: This compound was prepared as described in Example 1a except that N,N′-dimethyl-1,2-ethanediamine was substituted for piperazine. The final product was afforded as a pale yellow oil and 100% purity by LCMS (M+H=298.2) and NMR (3.21 g, 66% yield). ¹H NMR (DMSO-d6, 400 MHz) δ: 7.34 (1H, dd), 7.19 (1H, d), 7.16 (1H, d), 3.85 (3H, s), 3.83 (3H, s), 2.97 (2H, t), 2.66 (3H, s), 2.58 (2H, t), 2.26 (3H, s).

b). Preparation of N-methyl-N-[2-(methyl{[3-(methyloxy)phenyl]sulfonyl}amino)ethyl]-3,4-bis(methyloxy)benzenesulfonamide: This compound was prepared as described in Example 1 except that N-methyl-N-[2-(methylamino)ethyl]-3,4-bis(methyloxy)benzenesulfonamide, prepared as in Example 10a, was substituted for 1-{[3,4-bis(methyloxy)phenyl]sulfonyl}piperazine, and 3-(methyloxy)benzenesulfonyl chloride was substituted for 3-fluoro-4-(methyloxy)benzenesulfonyl chloride.

### Example 11

### N-{2-[{[3,4-bis(methyloxy)phenyl]sulfonyl}(methyl)amino]ethyl}-3-fluoro-N-methyl-4-(methyloxy)benzenesulfonamide

This compound was prepared as described in Example 1 except that N-methyl-N-[2-(methylamino)ethyl]-3,4-bis(methyloxy)benzenesulfonamide, prepared as in Example 10a, was substituted for 1-{[3,4-bis(methyloxy)phenyl]sulfonyl}piperazine.

### Example 12

### N-methyl-N-{2-[{[3,4-bis(methyloxy)phenyl]sulfonyl}(methyl)amino]ethyl}-4-methyl-3,4-dihydro-2H-1,4-benzoxazine-7-sulfonamide

This compound was prepared as described in Example 1 except that N-methyl-N-[2-(methylamino)ethyl]-3,4-bis(methyloxy)benzenesulfonamide, prepared as in Example 10a, was substituted for 1-{[3,4-bis(methyloxy)phenyl]sulfonyl}piperazine, and 4-methyl-3,4-dihydro-2H-1,4-benzoxazine-7-sulfonyl chloride was substituted for 3-fluoro-4-(methyloxy)benzenesulfonyl chloride.

### Example 13

### N-{2-[{[3,4-bis(methyloxy)phenyl]sulfonyl}(methyl)amino]ethyl}-N-methyl-2,3-dihydro-1,4-benzodioxin-6-sulfonamide

This compound was prepared as described in Example 1 except that N-methyl-N-[2-(methylamino)ethyl]-3,4-bis(methyloxy)benzenesulfonamide, prepared as in Example 10a, was substituted for 1-{[3,4-bis(methyloxy)phenyl]sulfonyl}piperazine, and 2,3-dihydro-1,4-benzodioxin-6-sulfonyl chloride was substituted for 3-fluoro-4-(methyloxy)benzenesulfonyl chloride.

### Example 14

### N,N′-1,2-ethanediylbis[N-methyl-3,4-bis(methyloxy)benzenesulfonamide]

This compound was prepared as described in Example 1 except that N-methyl-N-[2-(methylamino)ethyl]-3,4-bis(methyloxy)benzenesulfonamide, prepared as in Example 10a, was substituted for 1-{[3,4-bis(methyloxy)phenyl]sulfonyl}piperazine, and 3,4-bis(methyloxy)benzenesulfonyl chloride was substituted for 3-fluoro-4-(methyloxy)benzenesulfonyl chloride.

### Example 15

### N-methyl-N-[2-(methyl{[4-(methyloxy)phenyl]sulfonyl}amino)ethyl]-3,4-bis(methyloxy)benzenesulfonamide

This compound was prepared as described in Example 1 except that N-methyl-N-[2-(methylamino)ethyl]-3,4-bis(methyloxy)benzenesulfonamide, prepared as in Example 10a, was substituted for 1-{[3,4-bis(methyloxy)phenyl]sulfonyl}piperazine, and 4-(methyloxy)benzenesulfonyl chloride was substituted for 3-fluoro-4-(methyloxy)benzenesulfonyl chloride.

### Example 16

### N-(1-{[3,4-bis(methyloxy)phenyl]sulfonyl}-3-pyrrolidinyl)-8-quinolinesulfonamide

a). Preparation of 1,1-dimethylethyl (1-{[3,4-bis(methyloxy)phenyl]sulfonyl}-3-pyrrolidinyl)carbamate: To a solution of 1,1-dimethylethyl 3-pyrrolidinylcarbamate (5 g, 26.8 mmol) in 250 mL dichloromethane (DCM), was added diisopropylethylamine (6.9 g, 53.6 mmol), followed by 3,4-dimethoxysulfonyl chloride (6.3 g, 26.8 mmol). The reaction appeared complete by LCMS after 3 hours at room temperature. After stirring overnight, the reaction progress was unchanged by LCMS. The mixture was concentrated by half in vacuo. Water was added and the solution was acidified by addition of 1N HCl. The organic layer was washed with 1N HCl, saturated sodium bicarbonate solution, and brine. The organic layer was dried over magnesium sulfate (MgSO₄), filtered, concentrated, and dried in vacuo. The final product was afforded as a tan solid and 96% purity by LCMS (M+H=387.2) and NMR (7.99 g, 77% yield). ¹H NMR (CDCl₃, 400 MHz) δ: 7.45 (1H, dd), 7.28 (1H, d), 6.97 (1H, d), 4.5 (1H, m), 4.17 (1H, m), 3.96 (3H, s), 3.94 (3H, s), 3.35 (2H, m), 3.20 (2H, m), 1.75 (1H, m), 1.42 (9H, s).

b). Preparation of 1-{[3,4-bis(methyloxy)phenyl]sulfonyl}-3-pyrrolidinamine trifluoroacetate: To the 1,1-dimethylethyl (1-{[3,4-bis(methyloxy)phenyl]sulfonyl}-3-pyrrolidinyl)carbamate (7.99 g, 20.7 mmol) was slowly added a solution of 50% trifluoroacetic acid in dichloromethane (50 mL). The reaction appeared complete by LCMS after 20 minutes at room temperature. After 30 min, the reaction mixture was concentrated and dried in vacuo. The final product was afforded as a pale pink solid and 100% purity by LCMS (M+H=287.2) and NMR (12.32 g, excess mass due to residual TFA). ¹H NMR (DMSO-d6, 400 MHz) δ: 8.06 (br H), 7.39 (1H, dd), 7.21 (1H, br s), 7.19 (1H, s), 3.86 (3H, s), 3.85 (3H, s), 3.70 (1H, m), 3.34 (1H, ddt), 3.28 (1H, dd), 3.12 (2H, m), 2.04 (1H, m), 1.78 (1H, m), 1.26 (2H, dd).

c). Preparation of N-(1-{[3,4-bis(methyloxy)phenyl]sulfonyl}-3-pyrrolidinyl)-8-quinolinesulfonamide: This compound was prepared as described in Example 1 except that 1-{[3,4-bis(methyloxy)phenyl]sulfonyl}-3-pyrrolidinamine trifluoroacetate, prepared as in Example 16b, was substituted for 1-{[3,4-bis(methyloxy)phenyl]sulfonyl}piperazine, an additional equivalent of triethylamine in Example 1b was used to solubilize the trifluoroacetate salt, and 8-quinolinesulfonyl chloride was substituted for 3-fluoro-4-(methyloxy)benzenesulfonyl chloride.

### Example 17

### (2R,6R)-1,4-bis{[3,4-bis(methyloxy)phenyl]sulfonyl}-2,6-dimethylpiperazine

In a 4 mL vial, was combined (2R,6R)-2,6-dimethylpiperazine hydrochloride (60 umol) and 500 uL pyridine. 3,4-bis(methyloxy)benzenesulfonyl chloride (2.5 eq) was added. The vial was sealed and shaken at 80° C. After 44 h, the reaction mixture was concentrated in vacuo, and then resubjected to the reaction conditions with 3,4-bis(methyloxy)benzenesulfonyl chloride added first, followed by 500 uL pyridine. The vial was sealed and shaken at 80° C. for 6 h. The reaction mixture was concentrated in vacuo, dissolved in 700 uL DMSO, and purified using reverse-phase HPLC to afford the final product.

### Example 18

### 1,4-bis{[3,4-bis(methyloxy)phenyl]sulfonyl}-2,5-dimethylpiperazine

This compound was prepared as described in Example 17 except that 2,5-dimethylpiperazine was substituted for (2R,6R)-2,6-dimethylpiperazine hydrochloride.

### Example 19

### (2S)-1,4-bis{[3,4-bis(methyloxy)phenyl]sulfonyl}-2-methylpiperazine

This compound was prepared as described in Example 17 except that (2S)-2-methylpiperazine was substituted for (2R,6R)-2,6-dimethylpiperazine hydrochloride.

### Example 20

### 1,4-bis{[3,4-bis(methyloxy)phenyl]sulfonyl}-2,6-cis-dimethylpiperazine

This compound was prepared as described in Example 17 except that 2,6-cis-dimethylpiperazine was substituted for (2R,6R)-2,6-dimethylpiperazine hydrochloride.

### Example 21

### (2S,6S)-1,4-bis{[3,4-bis(methyloxy)phenyl]sulfonyl}-2,6-dimethylpiperazine

This compound was prepared as described in Example 17 except that (2S,6S)-2,6-dimethyl piperazine was substituted for (2R,6R)-2,6-dimethyl piperazine hydrochloride.

### Example 22

### trans-1,4-bis{[3,4-bis(methyloxy)phenyl]sulfonyl}-2,5-dimethylpiperazine

In a 4 mL vial, was combined trans-2,5-dimethylpiperazine (200 umol) and 1000 uL pyridine. 3,4-bis(methyloxy)benzenesulfonyl chloride (3 eq) was added. The vial was sealed and shaken at 80° C. After 21 h, the reaction mixture was concentrated in vacuo, dissolved in DMSO, and purified using reverse-phase HPLC to afford the final product.

### Example 23

### 1,3-bis{[3,4-bis(methyloxy)phenyl]sulfonyl}-2-imidazolidinone

To the solution of 2-imidazolidinone (0.1 g, 1.16 mmol) in DCM (10 mL) was added sodium hydride (0.058 g, 2.32 mmol). The reaction mixture was allowed to stir at rt for 10 minutes under nitrogen, followed by the addition of 3,4-dimethoxyphenyl sulfonyl chloride (0.7 g, 3.0 mmol). After stirring at rt overnight, the mixture was concentrated under nitrogen. The crude product was dissolved in DMSO (3 mL), filtered, and purified using reverse phase HPLC. The final product was afforded as a light brown solid and 100% pure by LCMS (0.2 g, 35.4% yield).

### Example 24

### 1,4-bis{[3,4-bis(methyloxy)phenyl]sulfonyl}-2-cyclohexylpiperazine

In a single well of a Robbins Flex-Chem System 96 well 2.5 mL reaction block was added 2-cyclohexylpiperazine (60 umol, 0.117 M solution in DCM). 3,4-dimethoxyphenyl sulfonyl chloride (90 umol, 0.067 M solution in DCM with 10% triethylamine). The block was sealed and rotated overnight at rt. The reaction was filtered, concentrated in vacuo, dissolved in 700 uL DMSO, and purified using reverse phase HPLC to afford the final product (5.5 mg, 9.5 umol, 15.8% yield).

### Example 25

### N-(1-{[3,4-bis(methyloxy)phenyl]sulfonyl}hexahydro-1H-azepin-3-yl)-3,4-bis(methyloxy)benzenesulfonamide

This compound was prepared as described in Example 24 except that hexahydro-1H-azepin-3-amine was substituted for 2-cyclohexylpiperazine.

### Example 26

### N,N′-1,2-ethanediylbis[3,4-bis(methyloxy)benzenesulfonamide]

To the solution of 1,2-ethanediamine (1.0 g, 16.7 mmol) in DCM (50 mL) was added triethylamine (4.5 mL, 33.3 mmol), followed by the addition of 3,4 bis(methyloxy)benzenesulfonyl chloride (7.9 g, 33.3 mmol) in DCM (20 mL). The resulting solution was stirred at rt overnight. Water (50 mL) was added to the solution. The organic layer was dried over magnesium sulfate (MgSO₄), filtered, concentrated, and dried in vacuo. The crude product was dissolved in DMSO (10 mL), filtered, and purified using reverse phase HPLC. The final product was afforded as a light brown solid and 100% pure by LCMS (3.0 g, 39% yield).

### Example 27

### 1,4-bis{[3,4-bis(methyloxy)phenyl]sulfonyl}-2,3-piperazinedione

To the solution of N-[2-({[3,4-bis(methyloxy)phenyl]sulfonyl}amino)ethyl]-3-methyl-4-(methyloxy)benzenesulfonamide from Example 32 (0.3 g, 0.65 mmol) in DCM with 10% triethylamine (10 mL) was added ethanedioyl dichloride (0.114 mL, 1.3 mmol) dropwise. The resulting solution was stirred at rt overnight. Water (20 mL) was added to the solution. The organic layer was dried over magnesium sulfate (MgSO₄), filtered, concentrated, and dried in vacuo. The crude product was dissolved in DMSO (3 mL), filtered, and purified using reverse phase HPLC. The final product was afforded as a light brown solid (0.020 g, 6% yield).

### Example 28

### 1,4-bis{[3,4-bis(methyloxy)phenyl]sulfonyl}hexahydro-5H-1,4-diazepin-5-one

This compound was prepared as described in Example 27 except that 3-chloropropanoyl chloride was substituted for ethanedioyl dichloride.

### Example 29

### 1,4-bis{[3,4-bis(methyloxy)phenyl]sulfonyl}-2-methylpiperazine

This compound was prepared as described in Example 24 except that 2-methylpiperazine was substituted for 2-cyclohexylpiperazine.

### Example 30

### 1-{[3,4-bis(methyloxy)phenyl]sulfonyl}-4-({4-(methyloxy)-3-[(trifluoromethyl)oxy]phenyl}sulfonyl)hexahydro-1H-1,4-diazepine

In a single well of a Robbins Flex-Chem System 96 well 2.5 mL reaction block was added 1-{[3,4-bis(methyloxy)phenyl]sulfonyl}hexahydro-1H-1,4-diazepine as prepared in Example 7 (90 umol, 1000 uL, 0.09 M solution in DCM) with 10% triethylamine. 4-(Methyloxy)-3-[(trifluoromethyl)oxy]benzenesulfonyl chloride (300 uL, 0.3 M solution in DCM) was added. The block was sealed and rotated overnight at room temperature. After 15 h, the reaction was filtered and collected by vacuum filtration. The reaction solids were washed with DCM (1×200 uL). The crude reaction mixture was concentrated in vacuo, dissolved in 700 uL DMSO, and purified using reverse-phase HPLC to afford the final product.

### Example 31

### N-methyl-N-{2-[methyl({4-(methyloxy)-3 [(trifluoromethyl)oxy]phenyl}sulfonyl)amino]ethyl}-3,4-bis(methyloxy)benzenesulfonamide

This compound was prepared as described in Example 30 except that N-methyl-N-[2-(methylamino)ethyl]-3,4-bis(methyloxy)benzenesulfonamide was substituted for 1-{[3,4-bis(methyloxy)phenyl]sulfonyl}hexahydro-1H-1,4-diazepine.

### Example 32

### 1-{[3,4-bis(methyloxy)phenyl]sulfonyl}-4-{[3-fluoro-4-(methyloxy)phenyl]sulfonyl}-2,6-cis-dimethylpiperazine

a) Preparation of 1,1-dimethylethyl 3,5-cis-dimethyl-1-piperazinecarboxylate: To a solution of 2,6-cis-dimethylpiperazine (2.8 g, 24.56 mmol) in DCM (45 mL) was added bis(1,1-dimethylethyl) dicarbonate (5.1 g, 25.37 mmol) at 0° C. The resulting reaction mixture was allowed to stir from 0° C. to rt over the period of 2.5 hours. Sodium bicarbonate solution (5% aqueous solution, 50 mL) was added to the reaction mixture. After stirring for another 10 minutes, the organic layer was dried over magnesium sulfate (MgSO₄), filtered through a Bond Elute™ silica gel column, and concentrated in vacuo. The final product was afforded as a white solid (3.5 g, 66.6% yield) (M+1=215.4), ¹H NMR (CDCl3, 400 MHz) δ: 3.95 (br 2H), 2.80 (br, 2H), 2.35 (br, 2H), 7.19 (1H, s), 1.46 (9H, s), 1.05 (dd, 6H, J=6.3 Hz).

b) Preparation of 1,1-dimethylethyl 4-{[3,4-bis(methyloxy)phenyl]sulfonyl}-3,5-cis-dimethyl-1-piperazinecarboxylate:

To a solution of 1,1-dimethylethyl-3,5-cis-dimethyl-1-piperazinecarboxylate (1.45 g, 6.78 mmol) in pyridine (60 mL) was added the solution of 3,4-bis(methyloxy)benzenesulfonyl chloride (1.43 g, 7.43 mmol) in DCM (20 mL) dropwise. After stirring at 80° C. overnight, the reaction mixture was concentrated in vacuo. The crude product was purified by chromatography on a silica gel column (ethyl acetate/hexane, gradient) to afford the desired product as colorless oil (3.8 g, 78.5% yield). (M+1=415.4), ¹H NMR (CDCl3, 400 MHz) δ: 7.27 (dd 1H), 7.26 (s, 1H), 6.93 (dd, 1H), 3.95 (s, 3H), 3.93 (s, 3H) 1.44 (s, 9H), 1.32 (dd, 6H, J=7.0 Hz).

c) Preparation of 1-{[3,4-bis(methyloxy)phenyl]sulfonyl}-2,6-cis-dimethylpiperazine trifluoroacetate: 1,1-Dimethylethyl 4-{[3,4-bis(methyloxy)phenyl]sulfonyl}-3,5-cis-dimethyl-1-piperazinecarboxylate was stirred in 50% trifluoroacetic acid in DCM for 0.5 h. The reaction solution was then concentrated in vacuo to give the desired product as a light brown solid. (M+1=315.2).

d) Preparation of 1-{[3,4-bis(methyloxy)phenyl]sulfonyl}-4-{[3-fluoro-4-(methyloxy)phenyl]sulfonyl}-2,6-cis-dimethylpiperazine: In a single well of a Robbins Flex-Chem System 96 well 2.5 mL reaction block was added 1-{[3,4-bis(methyloxy)phenyl]sulfonyl}-2,6-cis-dimethylpiperazine trifluoroacetate (60 umol, 0.067 M solution in pyridine), and 3-fluoro-4-(methyloxy)benzenesulfonyl chloride (60 umol, 0.2 M solution in pyridine). The block was sealed and rotated at 80° C. overnight. The reaction was filtered, concentrated in vacuo, dissolved in 700 uL DMSO, and purified using reverse phase HPLC to afford the final product.

### Example 33

### 1-{[3,4-bis(methyloxy)phenyl]sulfonyl}-2,6-cis-dimethyl-4-{[3-(methyloxy)phenyl]sulfonyl}piperazine

This compound was prepared as described in Example 32 except that 3-(methyloxy)benzenesulfonyl chloride was substituted for 3-fluoro-4-(methyloxy)benzenesulfonyl chloride.

### Example 34

### 1-{[3,4-bis(methyloxy)phenyl]sulfonyl}-2,6-cis-dimethyl-4-{[4-(1,3-oxazol-5-yl)phenyl]sulfonyl}piperazine

This compound was prepared as described in Example 32 except that 4-(1,3-oxazol-5-yl)benzenesulfonyl chloride was substituted for 3-fluoro-4-(methyloxy)benzenesulfonyl chloride.

### Example 35

### 1-{[3,4-bis(methyloxy)phenyl]sulfonyl}-2,6-cis-dimethyl-4-{[4-(methyloxy)phenyl]sulfonyl}piperazine

This compound was prepared as described in Example 32 except that 4-(methyloxy)benzenesulfonyl chloride was substituted for 3-fluoro-4-(methyloxy)benzenesulfonyl chloride.

### Example 36

### 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine

a) Preparation of 1-{[3,4-bis(methyloxy)phenyl]sulfonyl}-3,5-cis-dimethylpiperazine: To a solution of 2,6-cis-dimethylpiperazine (0.91 g, 7.98 mmol) in DCM (10 mL) at 0° C. was added triethylamine (1 mL), followed by addition of the solution of 3,4-bis(methyloxy)benzenesulfonyl chloride (1.72 g, 7.25 mmol) in DCM (20 mL) dropwise over the period of 10 minutes. After stirring from 0° C. to rt overnight, water (30 mL) was added. The organic layer was dried over magnesium sulfate (MgSO₄), filtered, and concentrated in vacuo to afforded the product as colorless oil (2.25 g, 98% yield). (M+1=315.2), ¹H NMR (CDCl3, 400 MHz) δ: 7.37 (dd 1H, J=2.1 Hz), 7.26 (s, 1H), 6.95 (dd, 1H, J=8.5 Hz), 3.93 (s, 3H), 3.92 (s, 3H), 3.62 (br, 2H), 3.00 (br, 2H), 1.85 (br, 2H), 1.03 (dd, 6H, J=6.2 Hz).

b) Preparation of 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine:

In an 8 mL test tube was added 1-{[3,4-bis(methyloxy)phenyl]sulfonyl}-3,5-cis-dimethylpiperazine (60 umol, 0.067 M solution in pyridine), and 4-(methyloxy)-3-[(trifluoromethyl)oxy]benzenesulfonyl chloride (60 umol, 0.2 M solution in pyridine). The test tube was sealed and shaken at 80° C. overnight. The reaction was filtered, concentrated in vacuo, dissolved in 700 uL DMSO, and purified using reverse phase HPLC to afford the final product.

### Example 37

### 6-[(4-{[3,4-bis(methyloxy)phenyl]sulfonyl}-2,6-cis-dimethyl-1-piperazinyl)sulfonyl]-2H-chromen-2-one

This compound was prepared as described in Example 42 except that 2-oxo-2H-chromene-6-sulfonyl chloride was substituted for 4-(methyloxy)-3-[(trifluoromethyl)oxy]benzenesulfonyl chloride

### Example 38

### 5-[(4-{[3,4-bis(methyloxy)phenyl]sulfonyl}-2,6-cis-dimethyl-1-piperazinyl)sulfonyl]-2-(methyloxy)phenol

a) Preparation of 5-[(−4-{[3,4-bis(methyloxy)phenyl]sulfonyl}-2,6-cis-dimethyl-1-piperazinyl)sulfonyl]-2-(methyloxy)phenyl methanesulfonate: This compound was prepared as described in Example 36 except that 5-(chlorosulfonyl)-2-(methyloxy)phenyl methanesulfonate (Intermediate Example 1) was substituted for 4-(methyloxy)-3-[(trifluoromethyl)oxy]benzenesulfonyl chloride. ¹H NMR (CDCl3, 400 MHz) δ: 7.62 (dd 1H,), 7.61 (s, 1H), 7.35 (dd, 1H), 7.18 (s, 1H), 7.08 (dd, 1H), 6.88 (dd, 1H,), 4.20 (br, 2H), 3.99 (s, 3H) 3.91 (s, 3H), 3.86 (s, 3H), 3.46 (br, 2H), 3.23 (s, 3H), 2.29 (br, 2H), 1.39 (dd, 6H).

b) Preparation of 5-[(−4-{[3,4-bis(methyloxy)phenyl]sulfonyl}-2,6-cis-dimethyl-1-piperazinyl)sulfonyl]-2-(methyloxy)phenol: 5-[(−4-{[3,4-bis(methyloxy)phenyl]sulfonyl}-2,6-cis-dimethyl-1-piperazinyl)sulfonyl]-2-(methyloxy)phenyl methanesulfonate (0.02 g, 0.035 mmol) was stirred in 5% KOH solution (33% water in isopropyl alcohol) (10 mL) in a sealed 25 mL tube at 100° C. overnight. The reaction mixture was extracted with DCM (3×10 mL). The organic layer was dried over magnesium sulfate (MgSO₄), filtered, concentrated, and dried in vacuo. The crude product was dissolved in DMSO (1.4 mL), filtered, and purified using reverse phase HPLC. The final product was afforded as a white solid.

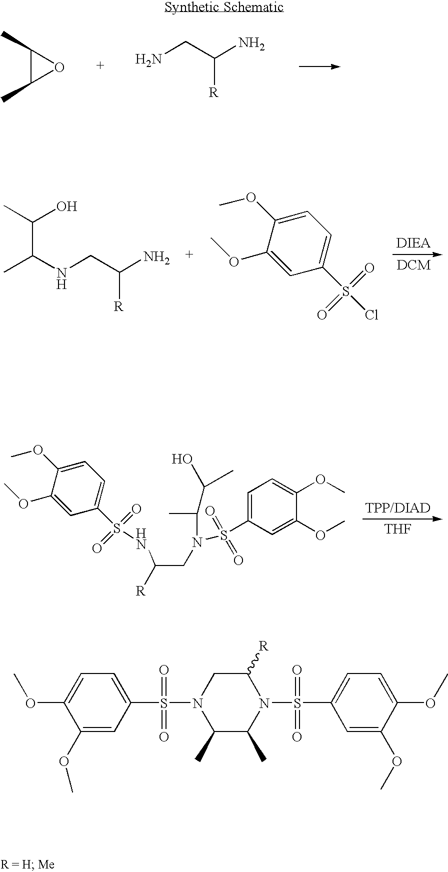

### Example 39

### 1,4-bis{[3,4-bis(methyloxy)phenyl]sulfonyl}-2,3,5-trimethylpiperazine

a) Preparation of N-[2-({[3,4-bis(methyloxy)phenyl]sulfonyl}amino)propyl]-N-(2-hydroxy-1-methylpropyl)-3,4-bis(methyloxy)benzenesulfonamide: Cis-2,3-dimethyloxirane 0.5 g (6.9 mmol) was dissolved in 2.06 g (27.2 mmol) of 1,2-propanediamine and stirred overnight at 100° C. in a sealed tube. Excess 1,2-propanediamine was removed under reduced pressure, and crude product (3-[(2-aminopropyl)amino]-2-butanol) was obtained: 1.01 g (87% yield). Next, 0.2 g (1.37 mmol) of 3-[(2-aminopropyl)amino]-2-butanol was dissolved in methylene chloride. 0.53 g of diisopropylethyl amine was added followed by 0.712 g (3.01 mmol) of 3,4 bis(methyloxy)benzenesulfonyl chloride, and the mixture was stirred overnight. Solvent was evaporated and product was purified by chromatography on SiO₂ with methylene chloride:ethyl acetate (100%:0% to 50%:50% gradient) yielding 0.6 g (80% yield) of N-[2-({[3,4-bis(methyloxy)phenyl]sulfonyl}amino)propyl]-N-(2-hydroxy-1-methylpropyl)-3,4-bis(methyloxy)benzenesulfonamide. Product contained a small amount of ethyl acetate as determined by NMR. ¹H NMR (400 MHz, CDCl₃+TFA): δ 7.55-7.45 (m, 2H); 7.40-7.30 (m, 2H), 6.98-6.90 (m, 2H); 3.96-3.92 (m, 12H); 3.86-2.72 (m, 5H); 1.16 (m, 3H); 0.98 (m, 3H); 0.77 (m, 3H). LCMS (M+H=547).

b) Preparation of 1,4-bis{[3,4-bis(methyloxy)phenyl]sulfonyl}-2,3,5-trimethylpiperazine: 0.144 g (0.55 mmol) of triphenylphosphine and 0.111 g (0.55 mmol) of diisopropyl azodicarboxylate were dissolved in 3 ml of dry THF at 0° C. and stirred for 30 min. 0.20 g (0.37 mmol) of N-[2-({[3,4-bis(methyloxy)phenyl]sulfonyl}amino)propyl]-N-(2-hydroxy-1-methylpropyl)-3,4-bis(methyloxy)benzenesulfonamide in 2 ml of dry THF was added dropwise, and the mixture was stirred overnight. Product was purified by chromatography on SiO₂ with methylene chloride:ethyl acetate (100%:0% to 50%:50% gradient). Subsequent crystallization from methanol provided 0.160 g (83% yield) of (2R,3S)-1,4-bis{[3,4-bis(methyloxy)phenyl]sulfonyl}-2,3,5-trimethylpiperazine. Product was a mixture of two diastereoisomers (4:6 ratio) ¹H NMR (400 MHz, CDCl₃) δ 7.41-7.30 (m, 2H); 7.26-7.18 (m, 2H); 6.94-6.86 (m, 2H); 5.00-4.93 (m, 0.4H); 4.52-4.44 (m, 0.4H); 4.14-4.06 (m, 1H); 3.96-3.88 (m, 12H); 3.84-3.77 (m, 1H); 3.63-3.57 (m, 0.6H); 3.48-3.42 (m, 0.4H); 3.34-3.28 (m, 0.4H); 3.26-3.18 (m, 0.6H); 3.10-3.04 (m, 0.6H); 1.34-1.20 (m, 9H). LCMS (M+H=529).

### Example 40

### 1,4-bis{[3,4-bis(methyloxy)phenyl]sulfonyl}-2,3-dimethylpiperazine

a) Preparation of N-[2-({[3,4-bis(methyloxy)phenyl]sulfonyl}amino)ethyl]-N-(2-hydroxy-1-methylpropyl)-3,4-bis(methyloxy)benzenesulfonamide: This compound was obtained according to the procedures for Example 39a except that 1,2-ethandiamine was substituted for 1,2-propanediamine. ¹H NMR (400 MHz, CDCl₃) δ 7.47 (d,d, 1H); 7.40 (d,d, 1H); 7.34 (d, 1H); 7.26 (d, 1H); 6.92 (d,d, 2H); 5.97-5.92 (m, 1H); 3.95-3.91 (m, 12H); 3.68-3.55 (m, 2H); 3.41-3.10 (m, 4H); 1.20 (d, J=6 Hz, 3H); 0.81 (d, 3H). LCMS (M+H=533).

b) Preparation of 1,4-bis{[3,4-bis(methyloxy)phenyl]sulfonyl}-2,3-dimethylpiperazine: N-[2-({[3,4-bis(methyloxy)phenyl]sulfonyl}amino)ethyl]-N-(2-hydroxy-1-methylpropyl)-3,4-bis(methyloxy)benzenesulfonamide was treated according to the procedure for Example 39b to give the title compound. ¹H NMR (400 MHz, CDCl₃) δ 7.34 (d, d, 2H); 7.18 (d, 2H); 6.92 (d, J=8 Hz, 2H); 3.95 (s, 6H); 3.91 (s, 6H); 3.66-3.58 (m, 2H); 3.42-3.36 (m, 2H); 3.24-3.17 (m, 2H); 1.17 (d, 6H). LCMS (M+H=515).

### Example 41

### 1-{[3,4-bis(methyloxy)phenyl]sulfonyl}-4-{[3-ethyl-4-(methyloxy)phenyl]sulfonyl}hexahydro-1H-1,4-diazepine; (Method A, Schematic 1)

a) Preparation of 3-ethyl-4-(methyloxy)benzenesulfonyl chloride: Under nitrogen atmosphere and anhydrous conditions, DMF-SO3 complex (4.51 g; 29 mmol) was suspended in dichloroethane (20 mL). 2-methyloxyethylbenzene (3.45 g; 25 mmol) was added in one portion and the mixture was heated at 75° C. for 2 h then allowed to cool to room temperature overnight. The solution was treated with oxalyl chloride (15.0 mL at 2.0 M in DCM; 30 mmol) added dropwise then heated at 65° C. for 4 h. The reaction was quenched by slow addition of 50 mL of water. The mixture was partitioned, and the organic phase was washed with water (2×50 mL), dried over Na2SO4, and concentrated in vacuo to give 6.03 g of 3-ethyl-4-methyloxybenzenesulfonyl chloride as a clear yellow oil. >99% purity by HPLC. LCMS ([(M-Cl)+OH]-=215). ¹H NMR (DMSO-d6) δ 7.39 (dd, 1H), 7.35 (d, 1H), 6.84 (d, 1H), 3.76 (s, 3H), 2.53 (q, 2H), 1.09 (t, 3H).

b) Preparation of 1-{[3,4-bis(methyloxy)phenyl]sulfonyl}-4-{[3-ethyl-4-methyloxyphenyl]sulfonyl}hexahydro-1H-1,4-diazepine: A solution of 1-{[3,4-bis(methyloxy)phenyl]sulfonyl}hexahydro-1H-1,4-diazepine (51 mg; 0.17 mmol) in 2 mL of DCM was treated with DIEA (36 mg; 0.28 mmol) followed by 3-ethyl-4-(methyloxy)benzenesulfonyl chloride (43 mg; 0.18 mmol). The solution was capped under nitrogen and allowed to stir overnight at room temperature. After 15 h, the reaction was diluted to 10 mL with DCM and washed once each with 5 mL of 1M NaHSO4, water, and sat'd aq. NaHCO3. The organic phase was dried over Na2SO4 and concentrated in vacuo to give 74 mg of the title compound as an amorphous solid. 95% purity by HPLC. LCMS (M+H=499). ¹H NMR (DMSO-d6) 67.57 (dd, 1H), 7.48 (d, 1H), 7.31 (dd, 1H), 7.15 (d, 1H), 7.10 (d, 1H), 7.09 (d, 1H), 3.82 (m, 13H), 3.17 (dd, 4H), 2.58 (q, 2H), 1.74 (m, 2H), 1.09 (t, 3H).

### Example 42

### N-[2-({[3,4-bis(methyloxy)phenyl]sulfonyl}amino)ethyl]-N-methyl-3,4-bis(methyloxy)benzenesulfonamide

A solution of N-methylethylenediamine (40 mg; 0.54 mmol) and DIEA (235 uL; 1.35 mmol) in 2 mL of DCM was treated with 3,4-dimethoxybenzenesulfonyl chloride (262 mg; 1.11 mmol). The reaction was capped under nitrogen and stirred at room temperature overnight. The resulting solution was diluted to 10 mL with DCM and washed once each with 5 mL of 1M NaHSO4, water, and sat'd aq. NaHCO3. The organic phase was dried over Na2SO4 and concentrated in vacuo to give 232 mg of the title compound as a white amorphous solid. 98% purity by HPLC. LCMS (M+H=475). ¹H NMR (DMSO-d6) δ 7.56 (t, 1H), 7.33 (dd, 1H), 7.25 (m, 2H), 7.10 (m, 3H), 3.82 (s, 3H), 3.81 (s, 3H), 3.79 (s, 3H), 3.79 (s, 3H), 2.91 (m, 2H), 2.83 (m, 2H), 2.60 (s, 3H).

### Example 43

### N-{2-[{[3,4-bis(methyloxy)phenyl]sulfonyl}(methyl)amino]ethyl}-N-methyl-3-ethyl-4-(methyloxy)benzenesulfonamide

A solution of N-methyl-N-[2-(methylamino)ethyl]-3,4-bis(methyloxy)benzenesulfonamide (50 mg; 0.17 mmol), prepared as Example 10a, and DIEA (46 uL; 0.26 mmol) in 2 mL of DCM was treated with 3-ethyl-4-(methyloxy)benzenesulfonyl chloride (43 mg; 0.18 mmol) (prepared as Example 41a). The solution was capped under nitrogen and allowed to stir at room temperature overnight. The reaction was diluted to 10 mL with DCM and washed once each with 5 mL of 1M NaHSO4, water, and sat'd aq. NaHCO3. The organic phase was dried over Na2SO4 and concentrated in vacuo to give 78 mg of the title compound as a white amorphous solid. 90% purity by HPLC. LCMS (M+H=487). 1H NMR (DMSO-d6) δ 7.58 (dd, 1H), 7.48 (d, 1H), 7.33 (dd, 1H), 7.17 (d, 1H), 7.14 (d, 1H), 7.13 (d, 1H), 3.86 (s, 3H), 3.83 (s, 3H), 3.61 (s, 3H), 3.05 (m, 4H), 2.66 (s, 3H), 2.63 (s, 3H), 2.60 (q, 2H), 1.11 (t, 3H).

### Example 44

### N,N′-1,2-propanediylbis[N-methyl-3,4-bis(methyloxy)benzenesulfonamide]

a) Preparation of N,N′-1,2-propanediylbis[3,4-bis(methyloxy)benzenesulfonamide]: To a solution of 1,2-diaminopropane (80 mg; 1.08 mmol) in 4 mL of DCM was added DIEA (0.47 mL; 2.7 mmol) followed by 3,4-dimethyloxybenzenesulfonyl chloride (524 mg; 2.21 mmol). The solution was capped under nitrogen and allowed to stir at room temperature overnight. The reaction was diluted to 10 mL with DCM and washed once each with 5 mL of 1M NaHSO4, water, and sat'd aq. NaHCO3. The organic phase was dried over Na2SO4 and concentrated in vacuo to give 533 mg of N,N′-1,2-propanediylbis[3,4-bis(methyloxy)benzenesulfonamide] as a white amorphous solid. 97% purity by HPLC. LCMS (M+H=475).473 [M−H]−. 1H NMR (DMSO-d6) δ 7.46 (t, 1H), 7.40 (d, 1H), 7.28 (dd, 1H), 7.23 (d, 1H), 7.21 (m, 2H), 7.04 (m, 2H), 3.82 (s, 3H), 3.81 (s, 3H), 3.77 (s, 3H), 3.76 (s, 3H), 3.08 (m, 1H), 2.62 (m, 1H), 2.43 (m, 1H), 0.84 (d, 3H).

b) Preparation of N,N′-1,2-propanediylbis[N-methyl-3,4-bis(methyloxy)benzenesulfonamide]: A solution of N,N′-1,2-propanediylbis[3,4-bis(methyloxy)benzenesulfonamide] (50 mg; 0.11 mmol) in 1.5 mL of CH3CN was treated with K2CO3 (86 mg; 0.63 mmol) followed by iodomethane (61 mg; 0.43 mmol). The mixture was stirred at room temperature overnight then briefly heated. The reaction was diluted to 10 mL with DCM and washed once each with 5 mL of 1M NaHSO4, water, and sat'd aq. NaHCO3. The organic phase was dried over Na2SO4 and concentrated in vacuo to give 47 mg of the title compound as a white crystalline solid. 94% purity by HPLC. LCMS (M+H=503). 1H NMR (DMSO-d6) δ 7.36 (dd, 1H), 7.32 (dd, 1H), 7.19 (d, 1H), 7.16 (d, 1H), 7.12 (d, 1H), 7.10 (d, 1H), 4.12 (m, 1H), 3.83 (s, 3H), 3.82 (s, 3H), 3.81 (s, 3H), 3.80 (s, 3H), 3.09 (m, 1H), 2.64 (s, 3H), 2.59 (s, 3H), 2.55 (m, 1H), 0.75 (d, 3H).

### Example 45

### (2R,5S)-1,4-bis{[3,4-bis(methyloxy)phenyl]sulfonyl}-2,5-diethylpiperazine

A mixture of (2R,5S)-2,5-diethylpiperazine hydrochloride (19 mg; 0.09 mmol) suspended in DCM (2 mL) was treated with DIEA (77 uL; 0.44 mmol) followed by 3,4-bis(methyloxy)benzenesulfonyl chloride (44 mg; 0.19 mmol). The resulting solution was capped under nitrogen and stirred at room temperature overnight. The reaction was diluted to 10 mL with DCM and washed once each with 5 mL of 1M NaHSO4, water, and sat'd aq. NaHCO3. The organic phase was dried over Na2SO4 and concentrated in vacuo to give 46 mg of crude product. The sample was triturated with CH3CN, and the supernatant was concentrated in vacuo to give 24 mg the title compound. 99% purity by HPLC. LCMS (M+H=543). ¹H NMR (DMSO-d6) δ 7.34 (dd, 2H), 7.17 (d, 2H), 7.08 (d, 2H), 3.80 (s, 6H), 3.78 (s, 6H), 3.71 (m, 2H), 3.48 (d, 2H), 3.01 (dd, 2H), 1.30 (m, 2H), 1.00 (m, 2H), 0.58 (t, 6H).

### Example 46

### 1,4-bis{[3,4-bis(methyloxy)phenyl]sulfonyl}-2-ethyl-5-methylpiperazine

This compound was prepared as described in Example 45 except that 2-ethyl-5-methylpiperazine hydrochloride was substituted for (2R,5S)-2,5-diethylpiperazine hydrochloride, and extractive workup provided 68 mg of sufficiently pure product. 91% purity by HPLC. LCMS (M+H=529). 1H NMR (DMSO-d6) δ 7.33 (m, 2H), 7.16 (m, 2H), 7.09 (m, 2H). 4.01 (bs, 1H), 3.79 (m, 12H), 3.12 (m, 1H), 2.99 (m, 1H), 1.43 (m, 1H), 1.11 (m, 1H), 0.67 (d, 3H), 0.62 (t, 3H).

### Example 47

### (2S,5S)-1,4-bis{[3,4-bis(methyloxy)phenyl]sulfonyl}-2,5-dimethylpiperazine

This compound was be prepared as described in Example 45 except that (2S, 5S)-2,5-dimethylpiperazine hydrobromide was substituted for (2R,5S)-2,5-diethylpiperazine hydrochloride. By this method, extractive workup provided 41 mg of sufficiently pure product. 93% purity by HPLC. LCMS (M+H=515). 1H NMR (DMSO-d6) δ 7.24 (dd, 2H), 7.07 (d, 2H), 7.03 (d, 2H). 3.82 (s, 6H), 3.75 (s, 6H), 3.37 (q, 2H), 3.12 (d, 4H), 1.12 (d, 6H).

This compound may be prepared from optically pure amino acids in the following way:

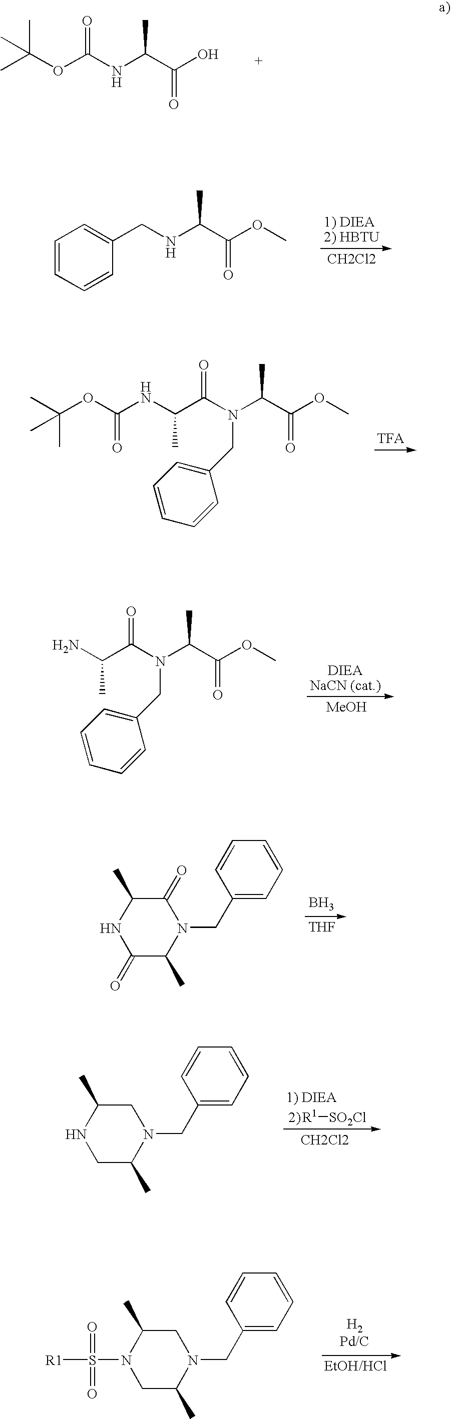

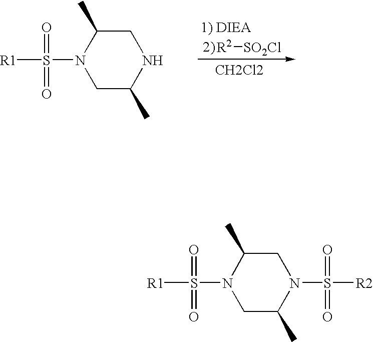

Preparation of methyl N-{[(1,1-dimethylethyl)oxy]carbonyl}-L-alanyl-N-(phenylmethyl)-L-alaninate: A mixture of N-Boc-L-alanine (0.73 g; 3.86 mmol) and N-benzyl-L-alanine methyl ester hydrochloride (0.83 g; 3.61 mmol) in 50 mL of DCM was treated with DIEA (1.04 g; 8.04 mmol) followed by O-benzotriazol-1-yl-N,N,N′,N′-tetramethyluronium hexafluorophosphate (1.51 g; 3.98 mmol). The resulting solution was capped under nitrogen and stirred at room temperature overnight. The reaction was diluted with ˜200 mL of CH₂Cl₂ and washed with 100 mL each of 1M NaHSO4, water, and sat'd NaHCO3. The organic phase was dried over Na2SO4 and concentrated in vacuo. The crude sample was taken up in 100 mL of EtOAc and washed with 3×25 mL of 1M NaHSO4. The organic phase was dried and conc. in vacuo., and the sample was applied to silica gel and purified by chromatography on 40 g of silica gel eluting with 2-30% CH2Cl2/EtOAc to give 0.691 g of N-{[(1,1-dimethylethyl)oxy]carbonyl}-L-alanyl-N-(phenylmethyl)-L-alaninate as a clear colorless resin contaminated with benzotriazole. 87% purity by HPLC. LCMS (M+H=365). 1H NMR (DMSO-d6) δ 7.35 (m, 4H), 7.25 (m, 1H), 4.65 (d, 2H), 4.34 (m, 1H), 4.25 (q, 1H), 3.51 (s, 3H), 1.32 (s, 9H), 1.17 (d, 3H), 1.10 (d, 3H).

b) Preparation of (3S,6S)-3,6-dimethyl-1-(phenylmethyl)-2,5-piperazinedione: A solution of N-{[(1,1-dimethylethyl)oxy]carbonyl}-L-alanyl-N-(phenylmethyl)-L-alaninate (0.69 g; </=1.89 mmol) in 15 mL of DCM was treated with 5 mL of TFA. The solution was stirred at room temperature for 30 min then concentrated in vacuo. The residue was twice taken up in 20 mL of methanol and concentrated in vacuo to give clear resin which was taken up in 500 mL of methanol. The solution was treated with DIEA until application to wet pH paper gave pH˜10, and a few crystals of NaCN were added. The reaction was allowed to stir overnight at room temperature, then it was conc. in vacuo. The residue was taken up in 75 mL of EtOAc and the solution was washed 3×25 mL with 1M NaHSO4, 2×25 mL with sat'd NaHCO3, and 1×25 mL with brine. The EtOAc was dried with Na2SO4 and concentrated in vacuo to give 0.303 g of (3S,6S)-3,6-dimethyl-1-(phenylmethyl)-2,5-piperazinedione as clear colorless resin. 98% purity by HPLC. LCMS (M+H=233). 1H NMR (DMSO-d6) δ 8.30 (bs, 1H), 7.33 (m, 2H), 7.25 (m, 3H), 4.92 (d, 1), 4.17 (d, 1H), 3.95 (m, 1H), 3.68 (q, 1H), 1.36 (d, 3H), 1.34 (d, 3H).

c) Preparation of (2S,5S)-2,5-dimethyl-1-(phenylmethyl)piperazine: Under nitrogen atmosphere and anhydrous conditions, (3S,6S)-3,6-dimethyl-1-(phenylmethyl)-2,5-piperazinedione (0.276 g; 1.19 mmol) in 10 mL of THF was treated with borane-THF (8.5 mL at 1.0 M; 8.5 mmol). The solution was heated at reflux for 4 h then treated with additional borane-THF (8.5 mL at 1.0 M) and heated for 3 hrs. The reaction was carefully quenched with ˜10 mL of methanol followed by ˜0.5 mL of conc. HCl. The quenched reaction was heated briefly to 70° C. then cooled and concentrated in vacuo. The concentrated sample was taken up in CH3CN and concentrated to give white solid which was triturated with diethyl ether. The supernatant was discarded and the remaining solid was dried in vacuo to give (2S,5S)-2,5-dimethyl-1-(phenylmethyl)piperazine hydrochloride as a white solid. 92% purity by HPLC. LCMS (M+H=205).

d) Preparation of (2S,5S)-1-{[3,4-bis(methyloxy)phenyl]sulfonyl}-2,5-dimethyl-4-(phenylmethyl)piperazine: A suspension of (2S,5S)-2,5-dimethyl-1-(phenylmethyl)piperazine hydrochloride (< or =1.19 mmol) in 20 mL of DCM was treated with DIEA (0.62 g; 4.8 mmol) followed by 3,4-dimethyloxybenzenesulfonyl chloride (0.31 g; 1.3 mmol). The resulting solution was capped under nitrogen and stirred at room temperature overnight. The reaction was diluted with 100 mL of CH2Cl2 and washed 3×50 mL with sat'd NaHCO3. The organic phase was dried with Na2SO4 and concentrated in vacuo. The crude product was applied to silica gel and purified by column chromatography on 40 g of silica gel eluting with 0-30% DCM/EtOAc to give 384 mg of (2S,5S)-1-{[3,4-bis(methyloxy)phenyl]sulfonyl}-2,5-dimethyl-4-(phenylmethyl)piperazine as clear colorless resin. 99% purity by HPLC. LCMS (M+H=405). 1H NMR (DMSO-d6) δ 7.34 (dd, 1H), 7.21 (m, 5H), 7.18 (d, 1H), 7.09 (d, 1H), 3.95 (d, 1H), 3.90 (bs, 1H), 3.82 (s, 3H), 3.78 (s, 3H), 3.52 (dd, 1), 2.92 (d, 1H), 2.77 (dd, 1H), 2.35 (dd, 1H), 2.09 (s, 1H), 1.90 (dd, 1H), 1.05 (d, 3H), 0.97 (d, 3H).

e) Preparation of (2S,5S)-1-{[3,4-bis(methyloxy)phenyl]sulfonyl}-2,5-dimethylpiperazine hydrochloride: Under a nitrogen atmosphere, a solution of (2S,5S)-1-{[3,4-bis(methyloxy)phenyl]sulfonyl}-2,5-dimethyl-4-(phenylmethyl)piperazine (0.357 g; 0.88 mmol) in 20 mL of absolute ethanol was treated with 1.00 M HCl (0.93 mL) followed by 10% Pd/C (˜20 mg). Hydrogen gas was introduced at atmospheric pressure, and the mixture was vigorously stirred at room temperature overnight. The reaction was filtered through Celite, and the filtrate was concentrated in vacuo. The resulting sample was taken up in absolute ethanol and concentrated to remove entrained water whereupon the product crystallized to give 302 mg of (2S,5S)-1-{[3,4-bis(methyloxy)phenyl]sulfonyl}-2,5-dimethylpiperazine hydrochloride as white crystalline solid. 100% purity by HPLC. LCMS (M+H=315). 1H NMR (DMSO-d6) δ 9.29 (bs, 0.5H), 8.59 (bs, 0.5H), 7.43 (dd, 1H), 7.26 (d, 1H), 7.14 (d, 1H), 4.24 (m, 1H), 3.85 (m, 1H), 3.83 (s, 3H), 3.82 (s, 3H), 3.12 (d, 1H), 2.95 (bs, 1H), 2.87 (m, 2H), 1.20 (d, 3H), 1.09 (d, 3H).

f) Preparation of (2S,5S)-1,4-bis{[3,4-bis(methyloxy)phenyl]sulfonyl}-2,5-dimethylpiperazine: A solution of (2S,5S)-1-{[3,4-bis(methyloxy)phenyl]sulfonyl}-2,5-dimethylpiperazine hydrochloride (20 mg; 0.057 mmol) in 4 mL of DCM was treated with DIEA (25 uL; 0.14 mmol) followed by 3,4-bis(methyloxy)benzenesulfonyl chloride (15 mg; 0.063 mmol). The solution was capped under nitrogen and stirred at room temperature for 3 days. The reaction was diluted to 10 mL with DCM and washed 2×5 mL with 1M NaHSO4, 1×5 mL with water, and 2×5 mL with sat'd aq. NaHCO3. The organic phase was dried and concentrated in vacuo to give 31 mg of crude product. The sample was applied to silica gel and purified by chromatography on 4 g of silica gel eluting with 0-20% EtOAc/DCM to give 18 mg of the title compound as white crystalline solid. 100% purity by HPLC, 98% enantiomeric purity by chiral SFC. LCMS (M+H=515). 1H NMR (DMSO-d6) δ 7.24 (dd, 2H), 7.08 (d, 2H), 7.03 (d, 2H), 3.82 (s, 6H), 3.75 (s, 6H), 3.36 (m, 2H), 3.12 (d, 4H), 1.12 (d, 6H).

### Example 48

### 1,4-bis{[3,4-bis(methyloxy)phenyl]sulfonyl}-5,5-dimethyl-2-piperazinone

a) Preparation of N-[2-({[3,4-bis(methyloxy)phenyl]sulfonyl}amino)-1,1-dimethylethyl]-3,4-bis(methyloxy)benzenesulfonamide: A solution of 2-methyl-1,2-propanediamine (0.080 g; 0.91 mmol) in 4 mL of DCM was treated with DIEA (0.40 mL; 2.27 mmol) followed by 3,4-bis(methyloxy)benzenesulfonyl chloride (0.44 g; 1.86 mmol). The resulting solution was capped under nitrogen and stirred at room temperature overnight. The reaction was diluted to 10 mL with DCM and washed once each with 5 mL of 1M NaHSO4, water, and sat'd aq. NaHCO3. The organic phase was dried over Na2SO4 and concentrated in vacuo to give 454 mg of N-[2-({[3,4-bis(methyloxy)phenyl]sulfonyl}amino)-1,1-dimethylethyl]-3,4-bis(methyloxy)benzenesulfonamide as a white amorphous solid. 95% purity by HPLC. LCMS (M+H=489). 1H NMR DMSO-d6) δ 7.41 (t, 1H), 7.31 (dd, 1H), 7.27 (d, 1H), 7.24 (m, 3H), 7.06 (d, 1H), 7.03 (d, 1H), 3.83 (s, 3H), 3.80 (s, 3H), 3.78 (s, 3H), 3.75 (s, 3H), 2.57 (d, 2H), 0.98 (s, 6H).

b) Preparation of 1,4-bis{[3,4-bis(methyloxy)phenyl]sulfonyl}-5,5-dimethyl-2-piperazinone: A solution of N-[2-({[3,4-bis(methyloxy)phenyl]sulfonyl}amino)-1,1-dimethylethyl]-3,4-bis(methyloxy)benzenesulfonamide (50 mg; 0.10 mmol) in 4 mL of DCM under nitrogen was treated with DIEA (54 uL; 0.31 mmol) followed by chloroacetyl chloride (9.2 uL; 0.11 mmol). After 15 h at room temperature, the reaction was treated with additional DIEA (40 uL) and chloroacetyl chloride (10 uL). After 72 h at room temperature, the reaction diluted to 10 mL with DCM and washed once each with 5 mL of 1M NaHSO4, water, and sat'd aq. NaHCO3. The organic phase was dried over Na2SO4 and concentrated in vacuo. The resulting crude product was applied to silica gel and purified by column chromatography using 4 g of silica gel and eluting with 20-80% EtOAc/hexane to give 41 mg of the title compound as a light tan crystalline solid. 100% purity by HPLC. LCMS (M+H=529). ¹H NMR (DMSO-d6) δ 7.51 (m, 1H), 7.31 (m, 2H), 7.13 (m, 1H), 7.03 (m, 2H), 4.07 (s, 2H), 3.87 (s, 2H), 3.84 (s, 3H), 3.81 (s, 3H), 3.76 (s, 3H), 3.73 (s, 3H), 1.36 (s, 6H).

### Example 49

### 1,4-bis{[3,4-bis(methyloxy)phenyl]sulfonyl}-2-methylhexahydro-1H-1,4-diazepine

A solution of N,N′-1,2-propanediylbis[3,4-bis(methyloxy)benzenesulfonamide] (25 mg; 0.05 mmol), prepared as described in Example 44a, in 2 mL of CH3CN was treated with K2CO3 (16 mg; 0.12 mmol) followed by 1,3-dibromopropane (11 mg; 0.05 mmol). The mixture was sealed and heated at 80° C. After starting material was consumed, the mixture was filtered, concentrated in vacuo, and purified by preparative HPLC to give 21 mg of the title compound as white amorphous solid. 98% by HPLC. LCMS (M+H=515). 1H NMR (DMSO-d6) δ 7.37 (dd, 1H), 7.32 (dd, 1H), 7.18 (m, 2H), 7.09 (m, 2H), 3.81 (m, 13H), 3.63 (m, 1H), 3.44 (m, 1H), 3.13 (m, 1H), 2.86 (m, 1H), 2.78 (m, 1H), 1.54 (m, 2H), 0.97 (d, 3H).

### Example 50

### 1,4-bis{[3,4-bis(methyloxy)phenyl]sulfonyl}-2,2-dimethylpiperazine

a) Preparation of 1,4-bis{[3,4-bis(methyloxy)phenyl]sulfonyl}-2,2-dimethyl-1,2,3,4-tetrahydropyrazine: Under nitrogen atmosphere and anhydrous conditions, a suspension of 1,4-bis{[3,4-bis(methyloxy)phenyl]sulfonyl}-5,5-dimethyl-2-piperazinone (50 mg; 0.10 mmol) in THF was treated with 1M borane-THF (0.29 mL; 0.29 mmol). The mixture was heated at 65° C. for 3 days then quenched by addition of methanol. The mixture was concentrated in vacuo then purified by column chromatography on 4 g of silica gel eluting with 0-20% EtOAc/DCM to provide 26 mg of 1,4-bis{[3,4-bis(methyloxy)phenyl]sulfonyl}-2,2-dimethyl-1,2,3,4-tetrahydropyrazine. 85% purity by HPLC. LCMS (M+H=513).

b) Preparation of 1,4-bis{[3,4-bis(methyloxy)phenyl]sulfonyl}-2,2-dimethylpiperazine: A solution of 1,4-bis{[3,4-bis(methyloxy)phenyl]sulfonyl}-2,2-dimethyl-1,2,3,4-tetrahydropyrazine (26 mg; 0.05 mmol) in 10 mL of ethanol and 2 mL of EtOAc was treated with 10% Pd/C (10 mg) followed by the introduction of hydrogen at atmospheric pressure. The mixture was stirred vigorously for 7 h. The mixture was filtered through Celite and a 0.2 um filter. The filtrate was concentrated in vacuo, and the product was crystallized from ethanol to give 14 mg of the title compound as a white powder. 95% purity by HPLC. LCMS (M+H=515). 1H NMR (DMSO-d6) δ 7.32 (dd, 1H), 7.27 (dd, 1H), 7.16 (d, 1H), 7.10 (d, 1H), 7.07 (m, 2H), 3.84 (s, 3H), 3.81 (s, 3H), 3.80 (s, 3H), 3.73 (s, 3H), 3.55 (m, 2H), 2.97 (m, 2H), 2.60 (bs, 2H), 1.16 (s, 6H).

### Example 51

### 1,4-bis{[3,4-bis(methyloxy)phenyl]sulfonyl}hexahydro-6H-1,4-diazepin-6-one

a) Preparation of N,N′-ethane-1,2-diylbis(3,4-dimethoxybenzenesulfonamide): To a cooled (0° C.) solution of 3,4-dimethoxybenzenesulfonyl chloride (3.37 g, 14.25 mmol), 30 mL DCM and diisopropylethylene diamine (5.2 mL, 29.9 mmol) was added dropwise 1,2-diaminoethane (2.0 mL, 29.9 mmol). The reaction was complete after warming to room temperature and stirring for 15 h. The solids were filtered off and discarded. The filtrate was collected and the product precipitated by washing with 1N HCl. The solids were collected and dried in vacuo at 35° C. The desired product was afforded as white solid (1.96 g, 30% yield). ¹H NMR (DMSO-d6, 400 MHz) δ: 7.25 (2 H, dd), 7.21 (2H, d), 7.05 (2H, d), 3.81 (6H, s), 3.76 (6H, s), 2.67 (4H, s).

b) Preparation of 1,4-bis[(3,4-dimethoxyphenyl)sulfonyl]-1,4-diazepan-6-ol: To a cooled (0° C.) solution of N,N′-ethane-1,2-diylbis(3,4-dimethoxybenzenesulfonamide) (1.96 g, 4.26 mmol) and solid potassium carbonate (5.87 g, 42.6 mmol) in 100 mL acetonitrile was added dropwise 1,3-dibromo-2-propanol (217 uL, 2.13 mmol). The reaction was allowed to warm to room temperature and then heated to 82° C. for 15 h. Additional 1,3-dibromo-2-propanol (109 uL, 1.07 mmol) was added and the reaction heated for and additional 48 h. The potassium carbonate was filtered off and the filtrate concentrated in vacuo to a clear oil, which turned to a solid upon standing. The desired product was utilized in this crude form (998 mg, 45% yield). LCMS (M+H=517.1) ¹H NMR (DMSO-d6, 400 MHz) δ: 7.32 (2H, dd), 7.17 (2H, d), 7.11 (2H, d), 5.23 (1H, d), 3.82 (6H, s), 3.80 (6H, s), 3.71 (1H, m), 3.46 (2H, d), 3.43 (2H, d), 3.08 (2H, m), 2.85 (2H, m).

c). Preparation of 1,4-bis{[3,4-bis(methyloxy)phenyl]sulfonyl}hexahydro-6H-1,4-diazepin-6-one. To a solution of 1,4-bis[(3,4-dimethoxyphenyl)sulfonyl]-1,4-diazepan-6-ol (125 mg, 0.24 mmol) in 15 mL DCM, was added silica bound pyridinium chlorochromate (1.25 g of 20% w/w). The suspension was sonicated briefly and then stirred at room temperature for 3 days. The resin was filtered off and filtrate concentrated in vacuo to an oil. The crude mixture was purified using reverse phase HPLC to afford the desired product as a foam (12.8 mg, 10% yield). LCMS (M+H=515.0) ¹H NMR (DMSO-d6, 400 MHz) δ: 7.36 (2H, dd), 7.17 (2H, d), 7.11 (2 H, d), 3.90 (4H, s), 3.82 (6H, s), 3.80 (6H, s), 3.53 (4H, s).

### Example 52

### 1,4-bis{[3,4-bis(methyloxy)phenyl]sulfonyl}-6-fluorohexahydro-1H-1,4-diazepine

To a solution of 1,4-bis[(3,4-dimethoxyphenyl)sulfonyl]-1,4-diazepan-6-ol (300 mg, 0.579 mmol) in 20 mL DCM was added diethylaminosulfurtrifluoride (153 uL, 1.16 mmol). After stirring for 2 h, 10 mL saturated sodium bicarbonate and 10 mL water were added. The organic phase was separated, dried over Na2SO4, filtered and concentrated in vacuo. The crude material was triturated with methanol and dried in vacuo. The desired product was afforded as a white solid (45.6 mg, 15% yield). LCMS (M+H=519.0) ¹H NMR (DMSO-d6, 400 MHz) δ: 7.35 (2H, dd), 7.20 (2 H, d), 7.11 (2H, d), 4.84 (1H, m), 3.82 (6H, s), 3.81 (6H, s), 3.57 (2H, m), 3.39 (4 H, m), 3.15 (2H, m). ¹⁹F NMR (DMSO-d6, 400 MHz) δ: −48.38 (s).

### Example 53

### 1,4-bis{[3,4-bis(methyloxy)phenyl]sulfonyl}-6,6-difluorohexahydro-1H-1,4-diazepine

a) Preparation of 1,4-bis{[3,4-bis(methyloxy)phenyl]sulfonyl}hexahydro-6H-1,4-diazepin-6-one: To a solution of 1,4-bis[(3,4-dimethoxyphenyl)sulfonyl]-1,4-diazepan-6-ol (323 mg, 0.625 mmol) in 45 mL DCM was added Des-Martin periodinane (402 mg, 0.95 mmol). After stirring for 1.5 h, the reaction was washed with aqueous sodium thiosulfate. The organics were dried over Na2SO4, filtered and concentrated in vacuo. The desired product was utilized in this crude form (254 mg, 79% crude yield). LCMS (M+H=515.0, M−H=513.1).

b) Preparation of 1,4-bis{[3,4-bis(methyloxy)phenyl]sulfonyl}-6,6-difluorohexahydro-1H-1,4-diazepine: To a solution of 4-bis{[3,4-bis(methyloxy)phenyl]sulfonyl}hexahydro-6H-1,4-diazepin-6-one (254 mg, 0.49 mmol) in 10 mL DCM was added (diethylamino)sulfur trifluoride (196 uL, 1.48 mmol). After stirring at room temperature for 5 h the reaction was diluted with DCM and saturated sodium bicarbonate. The organics were separated, dried over Na2SO4, filtered and concentrated in vacuo. The isolated material was triturated with methanol. The desired product was isolated as a white solid (157 mg, 60% yield). LCMS (M+H=537.0) ¹H NMR (DMSO-d6, 400 MHz) δ: 7.38 (2H, dd), 7.21 (2H, d), 7.11 (2H, d), 3.82 (6H, s), 3.81 (6H, s), 3.71 (4H, dd), 3.31 (4H, s). ¹⁹F NMR ((DMSO-d6, 400 MHz) δ: −100.57 (s).

### Example 54

### (2R,6S)-1,4-bis{[3,4-bis(methyloxy)phenyl]sulfonyl}-2,6-dimethylpiperazine

A solution of cis-2,6-dimethylpiperazine (50 mg, 1.1 mmol), 3,4-bis(methyloxy)benzenesulfonyl chloride (184 mg, 0.78 mmol) and diisopropylethyl amine (135 uL, 0.78 mmol) in 1.5 mL DCM was heated in a sealed vessel at reflux for 3 d. The resulting suspension was filtered and solids recovered, washing with methanol. The desired product was afforded as a white solid (37 mg, 16% yield). LCMS (M+H=515.0). ¹H NMR (DMSO-d6, 400 MHz) δ: 7.28 (1H, dd), 7.13 (1H, dd), 7.07 (1H, d), 7.06 (1H, d), 6.97 (1H, d), 6.95 (1H, d), 4.11 (2H, m), 3.84 (3H, s), 3.76 (6H, s), 3.65 (3H, s), 3.28 (2H, d), 1.77 (2H, m), 1.31 (3H, s), 1.29 (3H, s).

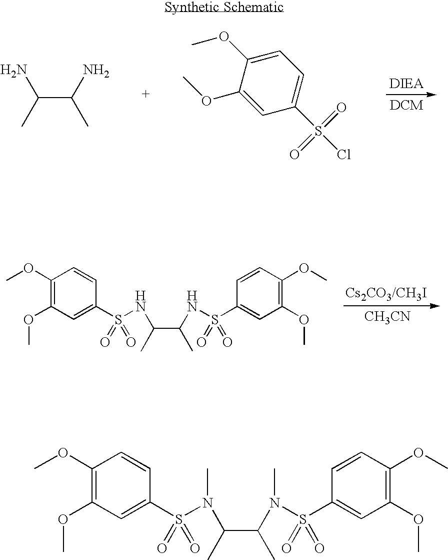

### Example 55

### N,N′-2,3-butanediylbis[N-methyl-3,4-bis(methyloxy)benzenesulfonamide]

a) Preparation of N,N′-2,3-butanediylbis[3,4-bis(methyloxy)benzenesulfonamide]: 0.6 g of (6.8 mM) of 2,3-butanediamine (obtained according to Robert M. Snapka, Sung Ho Woo, Andrei V. Blokhin and Donald T. Witiak Biochem Pharm, 52, 543 (1996)) was dissolved in methylene chloride, and 2.7 g (20.4 mM) of di-isopropylethylamine was added. The mixture was cooled to 0° C. and 1.93 g (8.17 mM) of 3,4-bis(methyloxy)benzenesulfonyl chloride was added. The mixture was stirred overnight. Solvent was removed under reduced pressure, and the product was purified by chromatography (SiO₂; Methylene chloride:Ethyl Acetate 100%:0% to 50%:50%) to provide 1 g (30% yield) of the title compound.

¹H NMR (400 MHz, CDCl₃) 7.48 (d-d, 2H); 7.35 (d, 2H); 6.92 (d, 2H); 4.80 (m, 2H); 3.92 (d, 12H); 3.22 (m, 2H); 0.94 (d, 6H). LCMS (M+H=489).

b) Preparation of N,N′-2,3-butanediylbis[N-methyl-3,4-bis(methyloxy)benzenesulfonamide]: 30 mg (0.06 mM) of N,N′-2,3-butanediylbis[3,4-bis(methyloxy)benzenesulfonamide were dissolved in acetonitrile and 60 mg (0.18 mM) of cesium carbonate was added. The mixture was cooled in an ice bath and 19 mg (0.14 mM) of methyl iodide was added. The mixture was stirred overnight and inorganic solid removed by filtration. Solvent was removed under reduced pressure and the product was purified by chromatography (SiO₂ Methylene chloride Ethyl Acetate 100%:0% to 50%:50%) to provide 14 mg (44% yield) of the title compound. ¹H NMR (400 MHz, CDCl₃) 7.47 (d, 2H); 7.25 (d, 2H); 6.03 (d, 2H); 4.03 (m, 2H); 3.94 (s, 12H)); 2.72 (s, 6H); 0.91 (d, 6H), LCMS (M+H=517).

### Example 56

### N-(1-{[3,4-bis(methyloxy)phenyl]sulfonyl}-3-pyrrolidinyl)-3,4-bis(methyloxy)-N-propylbenzenesulfonamide

a) Preparation of N-(1-{[3,4-bis(methyloxy)phenyl]sulfonyl}-3-pyrrolidinyl)-3,4-bis(methyloxy)benzenesulfonamide: To a solution of 3-aminopyrrolidine (694 mg, 8.05 mmol) and diisopropylethylamine (3.07 mL, 17.71 mmol) in 30 mL DCM was added 3,4-bis(methyloxy)benzenesulfonyl chloride (3.9 g, 16.52 mmol) in a portionwise manner. After 2 h the reaction solution was washed with 1 N HCl and saturated sodium bicarbonate solution. The organics were dried over Na2SO4, filtered and concentrated in vacuo. The desired product was afforded as a foam (3.92 g, 99% yield). LCMS (M+H=487.0). ¹H NMR (DMSO-d6, 400 MHz) δ: 7.74 (1 H, d), 7.30 (1H, dd), 7.28 (1H, dd), 7.23 (1H, d), 7.11-7.14 (2H, m), 7.08 (1H, d), 3.83 (3H, s), 3.81 (3H, s), 3.80 (3H, s), 3.77 (3H, s), 3.35 (1H, m), 3.16 (2H, m), 3.05 (1H, m), 2.88 (1H, m), 1.70 (1H, m), 1.48 (1H, m).

b) Preparation of N-(1-{[3,4-bis(methyloxy)phenyl]sulfonyl}-3-pyrrolidinyl)-3,4-bis(methyloxy)-N-propylbenzenesulfonamide: A suspension of N-(1-{[3,4-bis(methyloxy)phenyl]sulfonyl}-3-pyrrolidinyl)-3,4-bis(methyloxy)benzenesulfonamide (75 mg, 0.15 mmol), 1-iodopropane (30.14 uL, 0.30 mmol) and potassium carbonate (212 mg, 1.54 mmol) in 2 mL of acetonitrile was heated to reflux in a sealed vessel for 36 h. The suspension was cooled to room temperature and any solids filtered off. The filtrate was purified by reverse phase HPLC. The desired product was afforded as an oil (35.1 mg, 44% yield). LCMS (M+H=529.1). ¹H NMR (DMSO-d6, 400 MHz) δ: 7.31 (1H, dd), 7.28 (1H, dd), 7.13-7.17 (3H, m), 7.08 (1H, d), 4.22 (1H, p), 3.84 (3H, s), 3.83 (3H, s), 3.80 (3H, s), 3.77 (3H, s), 3.28 (1H, m), 3.07 (1H, dd), 2.72-2.95 (4H, m), 1.78 (1H, m), 1.40-1.59 (3H, m), 0.74 (3H, t).

### Example 57

### N-(1-{[3,4-bis(methyloxy)phenyl]sulfonyl}-3-pyrrolidinyl)-3,4-bis(methyloxy)-N-(2-methylpropyl)benzenesulfonamide

A suspension of N-(1-{[3,4-bis(methyloxy)phenyl]sulfonyl}-3-pyrrolidinyl)-3,4-bis(methyloxy)benzenesulfonamide (75 mg, 0.15 mmol), isobutyl iodide (36 uL, 0.30 mmol) and potassium carbonate (212 mg, 1.54 mmol) in 2 mL of acetonitrile was heated at 82° C. in a sealed vessel for 36 h. The suspension was cooled to room temperature and any solids filtered off. The filtrate was purified by reverse phase HPLC. The desired product was afforded as a oil (34 mg, 42% yield). LCMS (M+H=543.2). ¹H NMR (DMSO-d6, 400 MHz) δ: 7.30 (1H, dd), 7.27 (1H, dd), 7.12-7.17 (3 H, m), 7.08 (1H, d), 4.18 (1H, p), 3.84 (3H, s), 3.82 (3H, s), 3.81 (3H, s), 3.77 (3 H, s), 3.29 (1H, m), 3.07 (1H, m), 2.91 (1H, m), 2.75 (1H, m), 2.67 (2H, m), 1.70 (1H, m), 1.51 (1H, m), 0.77 (3H, d), 0.75 (3H, d).

### Example 58

### N-(1-{[3,4-bis(methyloxy)phenyl]sulfonyl}-3-pyrrolidinyl)-N-(cyclobutylmethyl)-3,4-bis(methyloxy)benzenesulfonamide

A suspension of N-(1-{[3,4-bis(methyloxy)phenyl]sulfonyl}-3-pyrrolidinyl)-3,4-bis(methyloxy)benzenesulfonamide (75 mg, 0.15 mmol), bromomethyl cyclobutane (35 uL, 0.30 mmol) and potassium carbonate (212 mg, 1.54 mmol) in 2 mL of acetonitrile was heated at 82° C. in a sealed vessel for 36 h. The suspension was cooled to room temperature and any solids filtered off. The filtrate was purified by reverse phase HPLC. The desired product was afforded as an oil (30 mg, 36% yield). LCMS (M+H=555.1). ¹H NMR (DMSO-d6, 400 MHz) δ: 7.29 (2H, m), 7.14-7.18 (2H, m), 7.12 (1H, d), 7.08 (1H, d), 4.18 (1H, p), 3.84 (3H, s), 3.82 (3H, s), 3.81 (3H, s), 3.77 (3H, s), 3.30 (1H, m), 3.08 (1H, dd), 2.87-2.97 (2H, m), 2.71 (1 H, dd), 2.33 (1H, m), 1.83 (2H, m), 1.62-1.77 (3H, m), 1.44-1.62 (3H, m).

### Example 59

### N-((3R)-1-{[3,4-bis(methyloxy)phenyl]sulfonyl}-3-pyrrolidinyl)-3,4-bis(methyloxy)-N-propylbenzenesulfonamide

a) Preparation of N-((3R)-1-{[3,4-bis(methyloxy)phenyl]sulfonyl}-3-pyrrolidinyl)-3,4-bis(methyloxy)benzenesulfonamide: To a solution of (3R)-3-pyrrolidinamine dihydrochloride (280 mg, 1.76 mmol) and triethylamine (1.10 mL, 7.92 mmol) in 15 mL DCM was added 3,4-bis(methyloxy)benzenesulfonyl chloride (811 mg, 3.43 mmol). After stirring for 1.5 h, the reaction solution was diluted with 1 N HCl. The organic phase was separated, dried over Na2SO4, filtered and concentrated in vacuo. The desired product was afforded as a foam (560 mg, 65% yield). LCMS (M+H=487.2). ¹H NMR (DMSO-d6, 400 MHz) δ: 7.73 (1H, d), 7.30 (1 H, dd), 7.28 (1H, dd), 7.23 (1H, d), 7.10-7.14 (2H, m), 7.08 (1H, d), 3.83 (3H, s), 3.81 (3H, s), 3.80 (3H, s), 3.77 (3H, s), 3.36 (1H, p), 3.12-3.20 (2H, m), 3.01-3.10 (1H, m), 1.70 (1H, m), 1.49 (1H, m).

b) Preparation of N-((3R)-1-{[3,4-bis(methyloxy)phenyl]sulfonyl}-3-pyrrolidinyl)-3,4-bis(methyloxy)-N-propylbenzenesulfonamide: A suspension of N-((3R)-1-{[3,4-bis(methyloxy)phenyl]sulfonyl}-3-pyrrolidinyl)-3,4-bis(methyloxy)benzenesulfonamide (50 mg, 0.10 mmol), 1-iodopropane (30 uL, 0.30 mmol) and potassium carbonate (70 mg, 0.5 mmol) in 2 mL of acetonitrile was heated to reflux in a sealed vessel for 15 h. The suspension was cooled to room temperature and any solids filtered off. The filtrate was purified by reverse phase HPLC. The desired product was afforded as an oil (27 mg, 52% yield). LCMS (M+H=529.2). ¹H NMR (DMSO-d6, 400 MHz) δ: 7.31 (1H, dd), 7.28 (1H, dd), 7.13-7.17 (3H, m), 7.08 (1H, d), 4.22 (1H, m), 3.84 (3H, s), 3.82 (3H, s), 3.80 (3H, s), 3.77 (3H, s), 3.28 (1H, m), 3.07 (1H, dd), 2.71-2.95 (4H, m), 1.78 (1H, m), 1.39-1.62 (3 H, m), 0.73 (3H, t).

### Example 60

### N-((3R)-1-{[3,4-bis(methyloxy)phenyl]sulfonyl}-3-pyrrolidinyl)-N-ethyl-3,4-bis(methyloxy)benzenesulfonamide

This compound was prepared as described in Example 59 except that iodoethane was substituted for 1-iodopropane. LCMS (M+H=515.2). ¹H NMR (DMSO-d6, 400 MHz) δ: 7.31 (1H, dd), 7.29 (1H, dd), 7.12-7.17 (3H, m), 7.08 (1H, d), 4.22 (1H, p), 3.84 (3H, s), 3.82 (3H, s), 3.81 (3H, s), 3.77 (3H, s), 3.29 (1H, m), 3.06 (1H, m), 2.98 (2H, m), 2.87 (1H, m), 2.77 (1H, m), 1.80 (1H, m), 1.58 (1 H, m), 1.08 (3H, t).

### Example 61

### N-((3S)-1-{[3,4-bis(methyloxy)phenyl]sulfonyl}-3-pyrrolidinyl)-3,4-bis(methyloxy)-N-propylbenzenesulfonamide

a) Preparation of N-((3S)-1-{[3,4-bis(methyloxy)phenyl]sulfonyl}-3-pyrrolidinyl)-3,4-bis(methyloxy)benzenesulfonamide: To a solution of (3S)-3-aminopyrrolidine (237 mg, 2.75 mmol) and triethylamine (843 uL, 6.05 mmol) in 9 mL DCM was added 3,4-bis(methyloxy)benzenesulfonyl chloride (1.27 g, 5.36 mmol). After stirring for 2 h, the reaction solution was diluted with 1 N HCl. The organic phase was separated, dried over Na2SO4, filtered and concentrated in vacuo. The desired product was afforded as a foam (560 mg, 65% yield). LCMS (M+H=487.2). ¹H NMR (DMSO-d6, 400 MHz) δ: 7.73 (1H, d), 7.30 (1H, dd), 7.28 (1H, dd), 7.23 (1 H, d), 7.10-7.14 (2H, m), 7.08 (1H, d), 3.83 (3H, s), 3.81 (3H, s), 3.80 (3H, s), 3.77 (3H, s), 3.36 (1H, sep), 3.12-3.20 (2H, m), 3.06 (1H, m), 2.88 (1H, m), 1.70 (1H, m), 1.48 (1H, m).

b) Preparation of N-((3S)-1-{[3,4-bis(methyloxy)phenyl]sulfonyl}-3-pyrrolidinyl)-3,4-bis(methyloxy)-N-propylbenzenesulfonamide: A suspension of N-((3S)-1-{[3,4-bis(methyloxy)phenyl]sulfonyl}-3-pyrrolidinyl)-3,4-bis(methyloxy)benzenesulfonamide (50 mg, 0.10 mmol), 1-iodopropane (30 uL, 0.30 mmol) and potassium carbonate (70 mg, 0.5 mmol) in 2 mL of acetonitrile was heated to reflux in a sealed vessel for 15 h. The suspension was cooled to room temperature and any solids filtered off. The filtrate was purified by reverse phase HPLC. The desired product was afforded as an oil (27 mg, 52% yield). LCMS (M+H=529.3). ¹H NMR (DMSO-d6, 400 MHz) δ: 7.31 (1H, dd), 7.28 (1H, dd), 7.13-7.17 (3H, m), 7.08 (1H, d), 4.22 (1H, m), 3.84 (3H, s), 3.82 (3H, s), 3.80 (3H, s), 3.77 (3H, s), 3.28 (1H, m), 3.07 (1H, dd), 2.71-2.95 (4H, m), 1.78 (1H, m), 1.39-1.62 (3 H, m), 0.73 (3H, t).

### Example 62

### N-((3S)-1-{[3,4-bis(methyloxy)phenyl]sulfonyl}-3-pyrrolidinyl)-N-ethyl-3,4-bis(methyloxy)benzenesulfonamide

This compound was prepared as described in Example 61 except that iodoethane was substituted for 1-iodopropane. LCMS (M+H=515.2). ¹H NMR (DMSO-d6, 400 MHz) δ: 7.31 (1H, dd), 7.29 (1H, dd), 7.12-7.17 (3H, m), 7.08 (1H, d), 4.22 (1H, p), 3.84 (3H, s), 3.82 (3H, s), 3.81 (3H, s), 3.77 (3H, s), 3.29 (1H, m), 3.06 (1H, m), 2.98 (2H, m), 2.87 (1H, m), 2.77 (1H, m), 1.80 (1H, m), 1.58 (1 H, m), 1.08 (3H, t).

### Example 63

### 4-[((2S,5S)-4-{[3,4-bis(methyloxy)phenyl]sulfonyl}-2,5-dimethyl-1-piperazinyl)sulfonyl]-2-(methyloxy)phenyl methanesulfonate

A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 4-(chlorosulfonyl)-2-(methyloxy)phenyl methanesulfonate (21 mg; 0.07 mmol) (Intermediate Example 1) The resulting solution was capped and stirred at room temperature for 3 days. The reaction was diluted to 10 mL with DCM and washed with 1M NaHSO4, water, sat'd aq. NaHCO3. The organic phase was dried with Na2SO4 and concentrated in vacuo to give 35 mg of the title compound. 96% purity by HPLC. LCMS (M+H=579). 1H NMR (DMSO-d6) δ 7.66 (dd, 1H), 7.57 (d, 1H), 7.31 (d, 1H), 7.27 (dd, 1H), 7.09 (d, 1H), 7.05 (d, 1H), 3.92 (s, 3H), 3.81 (s, 3H), 3.77 (s, 3H), 3.43 (m, 2H), 3.37 (s, 3H), 3.22 (m, 2H), 3.10 (m, 2H), 1.09 (d, 3H), 1.07 (d, 3H).

### Example 64

### 1-{[3,4-bis(methyloxy)phenyl]sulfonyl}-4-{[3-ethyl-4-(methyloxy)phenyl]sulfonyl}piperazine

A solution of 1-{[3,4-bis(methyloxy)phenyl]sulfonyl}piperazine (50 mg; 0.18 mmol), prepared as in Example 1a, in 2 mL of DCM was treated with DIEA (46 uL; 0.26 mmol) followed by 3-ethyl-4-(methyloxy)benzenesulfonyl chloride (43 mg; 0.18 mmol), prepared as in Example 41a. The solution was capped under nitrogen and stirred at room temperature overnight. The reaction was diluted to 10 mL with DCM and washed once each with 1M NaHSO4, water, and sat'd aq. NaHCO3. The organic phase was dried over Na2SO4 and concentrated in vacuo to give 79 mg of the title compound as a white crystalline solid. 97% purity by HPLC. LCMS (M+H=485). 1H NMR (DMSO-d6) δ 7.50 (dd, 1H), 7.38 (bs, 1H), 7.23 (dd, 1H), 7.10 (m, 2H), 7.03 (d, 1H), 3.87 (s, 3H), 3.83 (s, 3H), 3.78 (s, 3H), 2.93 (bs, 8H), 2.57 (q, 2H), 1.09 (t, 3H).

### Example 65

### N-methyl-N-{2-[{[3,4-bis(methyloxy)phenyl]sulfonyl}(methyl)amino]ethyl}-1,3-dimethyl-2-oxo-2,3-dihydro-1H-benzimidazole-5-sulfonamide

a) Preparation of 1,3-dimethyl-2-oxo-2,3-dihydro-1H-benzimidazole-5-sulfonyl chloride: Under nitrogen atmosphere and anhydrous conditions, chlorosulfonic acid (11.3 g; 97 mmol) was cooled to 0° C. and 1,3-dimethyl-1,3-dihydro-2H-benzimidazol-2-one (3.14 g; 19.4 mmol) in 5 mL of DCM was added slowly dropwise. The resulting solution was allowed to come to room temp. and stir under nitrogen for 2 days. The mixture was concentrated in vacuo then added slowly to crushed ice. The resulting aqueous suspension was filtered, and the residue was washed well with water and dried in vacuo to give 4.35 g of off-white powder. 98% purity by HPLC. LCMS (M+H=261). ¹H NMR: δ 7.34 (dd, 1H), 7.31 (d, 1H), 7.04 (d, 1H), 3.31 (s, 3H), 3.30 (s, 3H).

b) Preparation of N-methyl-N-{2-[{[3,4-bis(methyloxy)phenyl]sulfonyl}(methyl)amino]ethyl}-1,3-dimethyl-2-oxo-2,3-dihydro-1H-benzimidazole-5-sulfonamide: A solution of N-methyl-N-[2-(methylamino)ethyl]-3,4-bis(methyloxy)benzenesulfonamide (50 mg; 0.17 mmol), prepared as Example 10a, and DIEA (43 uL; 0.25 mmol) in 2 mL of DCM was treated with 1,3-dimethyl-2-oxo-2,3-dihydro-1H-benzimidazole-5-sulfonyl chloride (47 mg; 0.18 mmol). The solution was capped under nitrogen and stirred at room temperature overnight. The reaction was diluted to 10 mL with DCM and washed once each with 1M NaHSO4, water, and sat'd aq. NaHCO3. The organic phase was dried over Na2SO4 and concentrated in vacuo to give 87 mg of the title compound as white amorphous solid. 92% purity by HPLC. LCMS (M+H=513). 1H NMR (DMSO-d6) δ 7.49 (m, 2H), 7.32 (m, 2H), 7.14 (m, 2H), 3.82 (s, 3H), 3.80 (s, 3H), 3.38 (s, 3H), 3.36 (s, 3H), 3.07 (bs, 4H), 2.67 (s, 3H), 2.66 (s, 3H).

### Example 66

### N-[2-({[3,4-bis(methyloxy)phenyl]sulfonyl}amino)-1-methylethyl]-N-methyl-3,4-bis(methyloxy)benzenesulfonamide

a) Preparation of N²-{[3,4-bis(methyloxy)phenyl]sulfonyl}alaninamide: DL-Alaninamide hydrochloride: (0.500 g; 4.01 mmol) suspended in 20 mL of DCM was treated with DIEA (1.80 mL; 10.0 mmol) followed 3,4-bis(methyloxy)benzenesulfonyl chloride (0.997 g; 4.21 mmol). The mixture was capped under nitrogen and stirred at room temperature overnight. The mixture was diluted with DCM then washed once each with 1M NaHSO4, water, and sat'd aq. NaHCO3. The combined aqueous extracts were filtered, and the residue was washed well with water and dried in vacuo to give 0.756 g of N²-{[3,4-bis(methyloxy)phenyl]sulfonyl}alaninamide as a bright white powder. 92% purity by HPLC. LCMS (M+H=289).

b) Preparation of N²-{[3,4-bis(methyloxy)phenyl]sulfonyl}-N²-methylalaninamide: A solution of N²-{[3,4-bis(methyloxy)phenyl]sulfonyl}alaninamide (0.250 g; 0.87 mmol) in 20 mL of CH3CN was treated with K2CO3 (0.60 g; 4.3 mmol) followed by iodomethane (0.165 mL; 2.60 mmol). The mixture was sealed under nitrogen and heated at 40° C. overnight then diluted with CH3CN and DCM and filtered. The filtrate was concentrated in vacuo to give solid which was triturated with water. The mixture was filtered and the residue washed well with water and dried in vacuo to give 218 mg of N²-{[3,4-bis(methyloxy)phenyl]sulfonyl}-N²-methylalaninamide as a bright white powder. 100% purity by HPLC. LCMS (M+H=303).

c) Preparation of N-(2-amino-1-methylethyl)-N-methyl-3,4-bis(methyloxy)benzenesulfonamide hydrochloride: Under nitrogen atmosphere and anhydrous conditions, a solution of N²-{[3,4-bis(methyloxy)phenyl]sulfonyl}-N²-methylalaninamide (0.215 g; 0.71 mmol) in 10 mL of THF was treated with 1.0M borane-THF complex (4.3 mL). The resulting solution was heated at 65° C. for 4 h then treated with additional borane-THF (4.0 mL) and heated at 65° C. overnight. The cooled reaction was quenched by slow addition of methanol then concentrated in vacuo. The resulting solid was taken up in methanol, treated with 4 mL of 1M HCl and heated at 65° C. for 30 min. The cooled mixture was concentrated to an aqueous solution which was freeze-dried to give 301 mg of N-(2-amino-1-methylethyl)-N-methyl-3,4-bis(methyloxy)benzenesulfonamide hydrochloride as a yellow amorphous solid. 88% purity by HPLC. LCMS (M+H=289). 1H NMR (DMSO-d6) δ 7.84 (bs, 2-3H), 7.36 (dd, 1H), 7.19 (d, 1H), 7.16 (d, 1H), 4.06 (m, 1H), 3.83 (s, 3H), 3.82 (s, 3H), 2.79 (m, 2H), 2.63 (s, 3H), 0.68 (d, 3H).

d) Preparation of N-[2-({[3,4-bis(methyloxy)phenyl]sulfonyl}amino)-1-methylethyl]-N-methyl-3,4-bis(methyloxy)benzenesulfonamide: A solution of N-(2-amino-1-methylethyl)-N-methyl-3,4-bis(methyloxy)benzenesulfonamide hydrochloride (0.290 mg; 0.89 mmol) in 25 mL of DCM was treated with DIEA (0.60 mL; 3.4 mmol) followed by 3,4-bis(methyloxy)benzenesulfonyl chloride (236 mg; 0.98 mmol). The solution was capped under nitrogen and stirred at room temperature overnight. The reaction was diluted to 50 mL with DCM and washed once each with 1M NaHSO4, water, and sat'd aq. NaHCO3. The organic phase was dried over Na2SO4 and concentrated in vacuo to give crude product which was purified by column chromatography on 12 g of silica gel eluting with 0-30% EtOAc/DCM to give 274 mg of the title compound. 98% purity by HPLC. LCMS (M+H=489). 1H NMR (DMSO-d6) δ 7.52 (bt, 1H), 7.29 (m, 2H), 7.26 (d, 1H) 7.15 (d, 1H), 7.07 (d, 1H), 3.93 (m, 1H), 3.81 (s, 6H), 3.79 (s, 3H), 3.78 (s, 3H), 2.59 (m, 2H), 2.54 (s, 3H), 0.75 (d, 3H).

### Example 67

### (2S,5S)-1-{[3,4-bis(methyloxy)phenyl]sulfonyl}-4-{[3-fluoro-4-(methyloxy)phenyl]sulfonyl}-2,5-dimethylpiperazine

A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 3-fluoro-4-(methyloxy)benzenesulfonyl chloride (15 mg; 0.07 mmol). The resulting solution was capped and stirred at room temperature for 3 days. The reaction was diluted to 10 mL with DCM and washed with 1M NaHSO4, water, sat'd aq. NaHCO3. The organic phase was dried with Na2SO4 and concentrated in vacuo to give 32 mg of the title compound. 98% purity by HPLC. LCMS (M+H=503). 1H NMR (DMSO-d6) δ 7.51 (m, 2H), 7.26 (m, 2H), 7.05 (m, 2H), 3.91 (s, 3H), 3.81 (s, 3H), 3.76 (s, 3H), 3.40 (m, 2H), 3.15 (m, 3H), 1.10 (m, 6H).

### Example 68

### (2S,5S)-1-{[3,4-bis(methyloxy)phenyl]sulfonyl}-4-{[3-ethyl-4-(methyloxy)phenyl]sulfonyl}-2,5-dimethylpiperazine

A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 3-ethyl-4-(methyloxy)benzenesulfonyl chloride (16 mg; 0.07 mmol) prepared as in Example 41a. The resulting solution was capped and stirred at room temperature for 3 days. The reaction was diluted to 10 mL with DCM and washed with 1M NaHSO4, water, and sat'd aq. NaHCO3. The organic phase was dried with Na2SO4 and concentrated in vacuo to give 29 mg of the title compound. 95% purity by HPLC. LCMS (M+H=513). 1H NMR (DMSO-d6) δ 7.49 (dd, 1H), 7.40 (d, 1H), 7.25 (dd, 1H), 7.07 (d, 1H), 7.04 (d, 1H), 7.02 (d, 1H), 3.85 (s, 3H), 3.81 (s, 3H), 3.75 (s, 3H), 3.34 (m, 2H), 3.09 (m, 3H), 2.54 (q, 2H), 1.12 (m, 6H), 1.05 (t, 3H).

### Example 69

### 8-[((2S,5S)-4-{[3,4-bis(methyloxy)phenyl]sulfonyl}-2,5-dimethyl-1-piperazinyl)sulfonyl]quinoline

A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 8 quinolinesulfonyl chloride (16 mg; 0.07 mmol). The resulting solution was capped and stirred at room temperature for 3 days. The reaction was diluted to 10 mL with DCM and washed with 1M NaHSO4, water, and sat'd aq. NaHCO3. The organic phase was dried with Na2SO4 and concentrated in vacuo to give 12 mg of the title compound. 92% purity by HPLC. LCMS (M+H=506). 1H NMR (DMSO-d6) δ 8.91 (dd, 1H), 8.46 (dd, 1H), 8.29 (dd, 1H), 8.23 (dd, 1H), 7.68 (dd, 1H), 7.61 (dd, 1H), 7.17 (dd, 1H), 7.00 (d, 1H), 6.97 (d, 1H), 3.81 (s, 3H), 3.71 (s, 3H), 3.29-3.15 (m, 5H), 2.99 (dd, 1H), 1.16 (d, 3H), 0.99 (d, 3H).

### Example 70

### (2S,5S)-1-(1,3-benzodioxol-5-ylsulfonyl)-4-{[3,4-bis(methyloxy)phenyl]sulfonyl}-2,5-dimethylpiperazine

a) Preparation of 1,3-benzodioxole-5-sulfonyl chloride: Under nitrogen atmosphere and anhydrous conditions, DMF-SO3 complex (5.23 g; 34 mmol) was suspended in dichloroethane (20 mL). 1,3-benzodioxole (3.62 g; 30 mmol) in DCE (5 mL) was dropwise and the mixture was heated at 75° C. overnight. The solution was allowed to cool to room temperature and treated with oxalyl chloride (17.0 mL at 2.0 M in DCM; 34 mmol) added dropwise then heated at 65° C. for 5 h. The reaction was quenched by slow addition of 50 mL of water. The mixture was partitioned, and the organic phase was washed with water, dried over Na2SO4, and concentrated in vacuo to give 6.90 g of clear yellow oil which crystallized. The crude sample was triturated with hexane, and the solids were dried in vacuo to give 6.33 g of 1,3-benzodioxole-5-sulfonyl chloride as an off-white crystalline solid. 98% purity by HPLC. LCMS [(M-Cl)+OH=201]. 1H NMR (DMSO-d6) δ7.10 (dd, 1H), 7.01 (d, 1H), 6.80 (d, 1H), 5.99 (s, 2H).

b) Preparation of (2S,5S)-1-(1,3-benzodioxol-5-ylsulfonyl)-4-{[3,4-bis(methyloxy)phenyl]sulfonyl}-2,5-dimethylpiperazine: A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 1,3-benzodioxole-5-sulfonyl chloride (15 mg; 0.07 mmol). The resulting solution was capped and stirred at room temperature for 3 days. The reaction was diluted to 10 mL with DCM and washed 1M NaHSO4, water, and sat'd aq. NaHCO3. The organic phase was dried with Na2SO4 and concentrated in vacuo to give 31 mg of the title compound. 97% purity by HPLC. LCMS (M+H=499). ¹H NMR (DMSO-d6) δ 7.49 (dd, 1H), 7.40 (d, 1H), 7.25 (dd, 1H), 7.07 (d, 1H), 7.04 (d, 1H), 7.02 (d, 1H), 3.85 (s, 3H), 3.81 (s, 3H), 3.75 (s, 3H), 3.34 (m, 2H), 3.09 (m, 3H), 2.54 (q, 2H), 1.12 (m, 6H), 1.05 (t, 3H).

### Example 71

### (2S,5S)-1-{[3,4-bis(methyloxy)phenyl]sulfonyl}-4-(2,3-dihydro-1,4-benzodioxin-6-ylsulfonyl)-2,5-dimethylpiperazine

A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 2,3-dihydro-1,4-benzodioxin-6-sulfonyl chloride (15 mg; 0.06 mmol). The resulting solution was capped and stirred at room temperature for 3 days. The reaction was diluted to 10 mL with DCM and washed with 1M NaHSO4, water, and sat'd aq. NaHCO3. The organic phase was dried with Na2SO4 and concentrated in vacuo to give crude product which was purified by column chromatography on 4 g of silica gel eluting with 0-20% EtOAc/DCM to give 12 mg of the title compound. 100% purity by HPLC. LCMS (M+H=513). ¹H NMR (DMSO-d6) δ 7.27 (dd, 1H), 7.10 (m, 3H), 7.06 (d, 1H), 6.96 (d, 1H), 4.30 (m, 4H), 3.81 (s, 3H), 3.76 (s, 3H), 3.43 (m, 1H), 3.24 (m, 2H), 3.10 (m, 2H), 3.00 (dd, 1H), 1.11 (m, 6H).

### Example 72

### (2S,5S)-1-{[3,4-bis(methyloxy)phenyl]sulfonyl}-2,5-dimethyl-4-{[4-(methyloxy)phenyl]sulfonyl}piperazine

A mixture of (2S,5S)-1-{[3,4-bis(methyloxy)phenyl]sulfonyl}-2,5-dimethylpiperazine hydrochloride (20 mg; 0.06 mmol), prepared as in Example 47e, in 2 mL of DCM was treated with DIEA (30 uL; 0.17 mmol) followed by 4-(methyloxy)benzenesulfonyl chloride (14 mg; 0.07 mmol). The resulting solution was capped and stirred at room temperature for 3 days. The reaction was diluted to 10 mL with DCM and washed with 1M NaHSO4, water, and sat'd aq. NaHCO3. The organic phase was dried with Na2SO4 and concentrated in vacuo to give 34 mg of the title compound. 99% purity by HPLC. LCMS (M+H=485). 1H NMR (DMSO-d6) δ 7.58 (d, 2H), 7.27 (dd, 1H), 7.08 (d, 1H), 7.04 (d, 1H), 7.02 (d, 2H), 3.82 (s, 3H), 3.81 (s, 3H), 3.76 (s, 3H), 3.44 (m, 1H), 3.27-3.09 (m, 4H), 3.00 (dd, 1H), 1.11 (m, 6H.

### Example 73

### N-{2-[{[3,4-bis(methyloxy)phenyl]sulfonyl}(methyl)amino]ethyl}-N-methyl-3,4-dihydro-2H-1,5-benzodioxepin-7-sulfonamide

A solution of N-methyl-N-[2-(methylamino)ethyl]-3,4-bis(methyloxy)benzenesulfonamide (20 mg; 0.07 mmol), prepared as in Example 10a, in 4 mL of DCM was treated with DIEA (25 uL; 0.15 mmol) followed by 3,4-dihydro-2H-1,5-benzodioxepin-7-sulfonyl chloride (19 mg; 0.08 mmol). The solution was capped under N2 and stirred at room temperature for 3 days. The reaction was diluted to 10 mL with DCM and washed with 1M NaHSO4, water, and sat'd aq. NaHCO3. The organic phase was dried with Na2SO4 and concentrated in vacuo to give 37 mg of the title compound. 97% purity by HPLC. LCMS (M+H=501). 1H NMR (DMSO-d6) δ 7.32 (m, 2H), 7.25 (d, 1H), 7.14 (m, 3H), 4.22 (m, 4H), 3.83 (s, 3H), 3.81 (s, 3H), 3.06 (bs, 4H), 2.67 (s, 3H), 2.65 (s, 3H), 2.14 (m, 2H).

### Example 74

### N,N′-(2S)-1,2-propanediylbis[N-ethyl-3,4-bis(methyloxy)benzenesulfonamide]

a) Preparation of N,N′-(2S)-1,2-propanediylbis[3,4-bis(methyloxy)benzenesulfonamide]: The enantiomers of N,N′-1,2-propanediylbis[3,4-bis(methyloxy)benzenesulfonamide], prepared as in Example 44a, were resolved by chiral super-critical fluid chromatography to give N,N′-(2S)-1,2-propanediylbis[3,4-bis(methyloxy)benzenesulfonamide]. >98% purity and a single enantiomer by chiral super-critical fluid chromatography. Absolute configuration was determined by vibrational circular dichroism-infrared spectral analysis.

b) N,N′-(2S)-1,2-propanediylbis[N-ethyl-3,4-bis(methyloxy)benzenesulfonamide]: A solution of N,N′-(2S)-1,2-propanediylbis[3,4-bis(methyloxy)benzenesulfonamide] (15 mg; 0.03 mmol) in 2 mL of CH3CN was treated with K2CO3 (0.05 g; 0.4 mmol) followed by ethyl iodide (16 uL; 0.20 mmol). The mixture was heated at 70° C. and monitored by HPLC. After the r×n was complete, the mixture was diluted with CH2Cl2, filtered through Celite, and concentrated in vacuo. The sample was triturated with CH2Cl2, filtered through a 0.2 um filter, and concentrated in vacuo to give 17 mg of the title compound. 100% purity by HPLC. LCMS (M+H=531). ¹H NMR (DMSO-d6) δ 7.38 (dd, 1H), 7.31 (dd, 1H), 7.21 (m, 2H), 7.11 (d, 1H), 7.09 (d, 1H), 4.00 (m, 1H), 3.82 (s, 3H), 3.81 (s, 3H), 3.80 (s, 3H), 3.78 (s, 3H), 3.23-2.95 (m, 6H), 1.13 (t, 3H), 0.95 (d, 3H), 0.90 (t, 3H).

### Example 75

### N-{2-[{[3,4-bis(methyloxy)phenyl]sulfonyl}(2-buten-1-yl)amino]ethyl}-N-methyl-3,4-bis(methyloxy)benzenesulfonamide

a) Preparation of N-[2-({[3,4-bis(methyloxy)phenyl]sulfonyl}amino)ethyl]-N-2-buten-1-yl-3,4-bis(methyloxy)benzenesulfonamide: A solution of N,N′-1,2-ethanediylbis[3,4-bis(methyloxy)benzenesulfonamide] (50 mg; 0.11 mmol), prepared as in Example 26, in 2 mL of CH3CN was treated with K2CO3 (˜80 mg; ˜0.58 mmol) followed by 1,3-dibromobutane (26 mg; 0.12 mmol). The mixture was capped under N2 and heated at 80° C. overnight. The mixture was diluted with CH2Cl2, filtered, and concentrated in vacuo. The sample was purified by chromatography on silica eluting with 0-30% EtOAc/CH2Cl2 to give 36 mg of N-[2-({[3,4-bis(methyloxy)phenyl]sulfonyl}amino)ethyl]-N-2-buten-1-yl-3,4-bis(methyloxy)benzenesulfonamide. 97% purity by HPLC. LCMS (M+H=515). ¹H NMR (DMSO-d6) δ 7.53 (t, 1H); 7.28 (m, 3H); 7.10 (m, 3H); 5.53 (m, 1H); 5.16 (m, 1H); 3.82 (s, 6H); 3.79 (s, 6H); 3.62 (d, 2H); 2.89 (m, 2H); 2.78 (m, 2H); 1.54 (d, 3H).

b) Preparation of N-{2-[{[3,4-bis(methyloxy)phenyl]sulfonyl}(2-buten-1-yl)amino]ethyl}-N-methyl-3,4-bis(methyloxy)benzenesulfonamide: A solution of N-[2-({[3,4-bis(methyloxy)phenyl]sulfonyl}amino)ethyl]-N-2-buten-1-yl-3,4-bis(methyloxy)benzenesulfonamide (33 mg; 0.06 mmol) in 2 mL of CH3CN was treated with K2CO3 (40 mg; 0.13 mmol) followed by iodomethane (10 mg; 0.07 mmol). The mixture was capped under N2 and stirred at rt overnight. The reaction was then treated with additional iodomethane (10 mg; 0.07 mmol) and allowed to stir at rt for 2 days. The reaction was diluted with DCM, filtered, and concentrated in vacuo. The remaining solid was triturated with DCM and the supernatant was concentrated in vacuo to give 34 mg the title compound as light yellow crystalline solid. 95% purity by HPLC. LCMS (M+H=529). 1H NMR (DMSO-d6) δ 7.35 (dd, 1H); 7.29 (dd, 1H); 7.19 (d, 1H); 7.13 (m, 3H); 5.62 (m, 1H); 5.22 (m, 1H); 3.83 (s, 3H); 3.82 (s, 3H); 3.81 (s, 3H); 3.80 (s, 3H); 3.69 (bd, 2H); 3.14 (m, 2H); 3.00 (m, 2H); 2.63 (d, 3H); 1.58 (m, 3H).

### Example 76

### 1,4-bis{[3,4-bis(methyloxy)phenyl]sulfonyl}-2-piperazinone

To a suspension of N,N′-ethane-1,2-diylbis(3,4-dimethoxybenzenesulfonamide) (4.93 g, 10.72 mmol) in 150 mL acetonitrile, potassium carbonate (4.4 g, 32.16 mmol) and chloroacetyl chloride (0.896 mL, 11.25 mmol) were added. After stirring for 30 m additional potassium carbonate (15 g, 108.5 mmol) and chloroacetyl chloride (1.71 mL, 21.44 mmol) were added. The suspension was stirred overnight at room temperature. The solids were filtered off and the filtrate concentrated under vacuo to an oil. The oil was redissolved in DCM and washed with 1 N NaOH (2×100 mL) and 1 N HCl (2×100 mL), dried over Na2SO4, filtered and concentrated to solids. The solids were triturated with methanol, collected and dried under vacuum. LCMS (M+H=501.2). ¹H NMR (DMSO-d6, 400 MHz) δ: 7.46 (1H, dd), 7.34 (1H, dd), 7.30 (1H, d), 7.12 (1H, d), 7.11 (1H, d), 7.09 (1H, d), 3.84 (9H, m), 3.77 (7H, m), 3.41 (2H, t).

### Example 77

### N-((3R)-1-{[3,4-bis(methyloxy)phenyl]sulfonyl}-3-pyrrolidinyl)-3,4-bis(methyloxy)benzenesulfonamide

To a solution of (3R)-3-pyrrolidinamine dihydrochloride (280 mg, 1.76 mmol) and triethylamine (1.10 mL, 7.92 mmol) in 15 mL DCM was added 3,4-bis(methyloxy)benzenesulfonyl chloride (811 mg, 3.43 mmol). After stirring for 1.5 h the reaction solution was diluted with 1 N HCl. The organic phase was separated, dried over Na2SO4, filtered and concentrated in vacuo. The desired product was afforded as a foam (560 mg, 65% yield). LCMS (M+H=487.2). ¹H NMR (DMSO-d6, 400 MHz) δ: 7.73 (1H, d), 7.30 (1H, dd), 7.28 (1H, dd), 7.23 (1H, d), 7.10-7.14 (2 H, m), 7.08 (1H, d), 3.83 (3H, s), 3.81 (3H, s), 3.80 (3H, s), 3.77 (3H, s), 3.36 (1 H, p), 3.12-3.20 (2H, m), 3.01-3.10 (1H, m), 1.70 (1H, m), 1.49 (1H, m).

### Example 78

### 1-{[3,4-bis(methyloxy)phenyl]sulfonyl}-4-(2,3-dihydro-1,4-benzodioxin-6-ylsulfonyl)piperazine

This compound was prepared as described in Example 1 except that 2,3-dihydro-1,4-benzodioxin-6-sulfonyl chloride was substituted for 3-fluoro-4-(methyloxy)benzenesulfonyl chloride.

### Example 79

### cis-1,4-bis{[3,4-bis(methyloxy)phenyl]sulfonyl}-2,6-dimethylpiperazine

This compound was prepared as described in Example 32 except that 3,4-dimethyloxybenzenesulfonyl chloride was substituted for 3-fluoro-4-(methyloxy)benzenesulfonyl chloride, and that the final step was done in individual glass vessel instead of the Robbins Flex-Chem. System.

### Example 80

### 1-{[3,4-bis(methyloxy)phenyl]sulfonyl}-4-({4-(methyloxy)-3-[(trifluoromethyl)oxy]phenyl}sulfonyl)piperazine

This compound was prepared as described in Example 30 except that 1-{[3,4-bis(methyloxy)phenyl]sulfonyl}piperazine was substituted for 1-{[3,4-bis(methyloxy)phenyl]sulfonyl}hexahydro-1H-1,4-diazepine.

### Example 81

### N-{2-[{[3,4-bis(methyloxy)phenyl]sulfonyl}(cyclopropyl)amino]ethyl}-N-methyl-3,4-bis(methyloxy)benzenesulfonamide

A suspension of N-[2-({[3,4-bis(methyloxy)phenyl]sulfonyl}amino)ethyl]-N-cyclopropyl-3,4-bis(methyloxy)benzenesulfonamide: (40 mg, 0.08 mmol), iodomethane (10 uL, 0.16 mmol) and potassium carbonate (110 mg, 0.8 mmol) in 2 mL of acetonitrile was heated to 150° C. for 10 m in a sealed vessel using a Emrys Optimizer microwave from Personal Chemistry. The suspension was cooled to room temperature and any solids filtered off. The filtrate was purified by silica gel flash chromatography using 30% EtOAc/DCM. The desired product was afforded as a oil (38 mg, 93% yield). LCMS (M+H=515.0). ¹H NMR (DMSO-d6, 400 MHz) δ: 7.38 (1 H, dd), 7.30 (1H, dd), 7.21 (1H, d), 7.15 (1H, d), 7.13 (2H, m), 3.83 (3H, s), 3.81 (3H, s), 3.23 (2H, t), 3.06 (2H, t), 2.66 (3H, s), 2.06 (1H, m), 0.75 (2H, m), 0.66 (2 H, m).

### Example 82

### N-{2-[{[3,4-bis(methyloxy)phenyl]sulfonyl}(cyclopropyl)amino]ethyl}-N-(1-methylethyl)-3,4-bis(methyloxy)benzenesulfonamide

A suspension of N-[2-({[3,4-bis(methyloxy)phenyl]sulfonyl}amino)ethyl]-N-cyclopropyl-3,4-bis(methyloxy)benzenesulfonamide (26 mg, 0.052 mmol), 2-iodopropane (6.2 uL, 0.10 mmol) and potassium carbonate (72 mg, 0.52 mmol) in 2 mL of acetonitrile was heated to reflux in a sealed vessel for 18 h. The suspension was cooled to room temperature and any solids filtered off. The filtrate was purified by silica gel flash chromatography using 30% EtOAc/DCM. The desired product was afforded as a oil (19.7 mg, 72% yield). LCMS (M+H=543.2). ¹H NMR (DMSO-d6, 400 MHz) δ: 7.39 (1H, dd), 7.35 (1H, dd), 7.24 (1H, d), 7.21 (1H, d), 7.19 (1H, d), 7.11 (1H, d), 3.99 (1H, m), 3.85 (3H, s), 3.82 (3H, s), 3.81 (3H, s), 3.79 (3H, s), 3.23 (2H, m), 3.13 (2H, m), 2.16 (1H, m), 0.91 (6H, d), 0.78 (2H, m), 0.71 (2H, m).

### Example 83

### N-{2-[{[3,4-bis(methyloxy)phenyl]sulfonyl}(cyclopropyl)amino]ethyl}-N-ethyl-3,4-bis(methyloxy)benzenesulfonamide

A suspension of N-[2-({[3,4-bis(methyloxy)phenyl]sulfonyl}amino)ethyl]-N-cyclopropyl-3,4-bis(methyloxy)benzenesulfonamide (26 mg, 0.052 mmol), iodoethane (5.0 uL, 0.10 mmol) and potassium carbonate (72 mg, 0.52 mmol) in 2 mL of acetonitrile was heated to reflux in a sealed vessel for 18 h. The suspension was cooled to room temperature and any solids filtered off. The filtrate was purified by silica gel flash chromatography using 30% EtOAc/DCM. The desired product was afforded as a oil (20.0 mg, 74% yield). LCMS (M+H=529.2). ¹H NMR (DMSO-d6, 400 MHz) δ: 7.37 (1H, dd), 7.33 (1H, dd), 7.21 (1H, d), 7.18 (1H, d), 7.16 (1H, d), 7.12 (1H, d), 3.84 (3H, s), 3.83 (3H, s), 3.81 (3H, s), 3.80 (3H, s), 3.17 (4H, m), 3.13 (2H, q), 2.08 (1H, m), 1.02 (3H, t), 0.75 (2H, m), 0.68 (2H, m).

### Example 84

### 4-{[3,4-bis(methyloxy)phenyl]sulfonyl}-2,6-cis-dimethyl-1-{[3-(methyloxy)phenyl]sulfonyl}piperazine

This compound was prepared as described in Example 36 except that 3-(methyloxy)benzenesulfonyl chloride was substituted for 4-(methyloxy)-3-[(trifluoromethyl)oxy]benzenesulfonyl chloride.

### Example 85

### N-(1-{[3,4-bis(methyloxy)phenyl]sulfonyl}hexahydro-1H-azepin-3-yl)-N-methyl-3,4-bis(methyloxy)benzenesulfonamide

In an 8 mL vial, was combined N-methylhexahydro-1H-azepin-3-amine (80 umol) and 3,4-bis(methyloxy)benzenesulfonyl chloride (57 mg) in 1000 uL pyridine. The vial was sealed and shaken at 80° C. over the weekend. The reaction mixture was then concentrated in vacuo, dissolved in DMSO, and purified using reverse-phase HPLC to afford the final product.

### Example 86

### N-(1-{[3,4-bis(methyloxy)phenyl]sulfonyl}-3-pyrrolidinyl)-N-methyl-3,4-bis(methyloxy)benzenesulfonamide

This compound was prepared as described in Example 85 except that N-methyl-3-pyrrolidinamine was substituted for N-methylhexahydro-1H-azepin-3-amine.

### Example 87

### N-(1-{[3,4-bis(methyloxy)phenyl]sulfonyl}-3-pyrrolidinyl)-N-ethyl-3,4-bis(methyloxy)benzenesulfonamide

This compound was prepared as described in Example 85 except that N-ethyl-3-pyrrolidinamine was substituted for N-methylhexahydro-1H-azepin-3-amine.

### Example 88

### N,N′-1,2-ethanediylbis[N-ethyl-3,4-bis(methyloxy)benzenesulfonamide]

This compound was prepared as described in Example 85 except that N,N′-diethyl-1,2-ethanediamine was substituted for N-methylhexahydro-1H-azepin-3-amine.

### Example 89

### 1,4-bis{[3,4-bis(methyloxy)phenyl]sulfonyl}-1,2,3,4-tetrahydroquinoxaline

This compound was prepared as described in Example 85 except that 1,2,3,4-tetrahydroquinoxaline was substituted for N-methylhexahydro-1H-azepin-3-amine.

### Example 90

### N-((3S)-1-{[3,4-bis(methyloxy)phenyl]sulfonyl}-3-pyrrolidinyl)-N-methyl-3,4-bis(methyloxy)benzenesulfonamide

This compound was prepared as described in Example 61 except that iodomethane was substituted for 1-iodopropane. LCMS (M+H=501.2). ¹H NMR (DMSO-d6, 400 MHz) δ: 7.29 (1H, dd), 7.27 (1H, dd), 7.13 (4H, m), 4.29 (1H, m), 3.84 (3H, s), 3.83 (3H, s), 3.80 (3H, s), 3.78 (3H, s), 3.23 (1H, m), 3.05 (1H, m), 2.89 (1H, m), 2.81 (1H, m), 2.52 (3H, s), 1.71 (1H, m), 1.56 (1H, m).

### Example 91

### N-((3R)-1-{[3,4-bis(methyloxy)phenyl]sulfonyl}-3-pyrrolidinyl)-N-methyl-3,4-bis(methyloxy)benzenesulfonamide

This compound was prepared as described in Example 85 except that (3R)—N-methyl-3-pyrrolidinamine was substituted for N-methylhexahydro-1H-azepin-3-amine.

### Example 92

### (1S,4S)-2,5-bis{[3,4-bis(methyloxy)phenyl]sulfonyl}-2,5-diazabicyclo[2.2.1]heptane

This compound was prepared as described in Example 85 except that (1S,4S)-2,5-diazabicyclo[2.2.1]heptane was substituted for N-methylhexahydro-1H-azepin-3-amine.

### Example 93

### N-(1-{[3,4-bis(methyloxy)phenyl]sulfonyl}-3-piperidinyl)-N-ethyl-3,4-bis(methyloxy)benzenesulfonamide

a) Preparation of N-(1-{[3,4-bis(methyloxy)phenyl]sulfonyl}-3-piperidinyl)-3,4-bis(methyloxy)benzenesulfonamide: A solution of 3-aminopiperidine dihydrochloride (268 mg, 1.55 mmol), 3,4-bis(methyl)oxybenzenesulfonyl chloride (752 mg, 3.17 mmol) and DIEA (1.13 mL, 6.51 mmol) in 25 mL DCM was stirred at room temperature for 2 h. The reaction was washed with 1 N HCl followed by saturated bicarbonate solution. The organics were separated, dried over Na2SO4, filtered and concentrated in vacuo. The desired product was afforded as white solids (470 mg, 61% yield). LCMS (M+H=486.9, M−H=485.2). ¹H NMR (DMSO-d6, 400 MHz) δ: 7.66 (1H, d), 7.36 (1H, dd), 7.31 (1H, d), 7.17 (1H, dd), 7.11 (2H, m), 7.02 (1H, d), 3.83 (6H, s), 3.79 (3H, s), 3.30 (2H, m), 3.00 (1H, m), 2.24 (1H, m), 2.03 (1H, m), 1.62 (1H, m), 1.46 (1H, m), 1.31 (1H, m), 1.01 (1 h, M).

b) Preparation of N-(1-{[3,4-bis(methyloxy)phenyl]sulfonyl}-3-piperidinyl)-N-ethyl-3,4-bis(methyloxy)benzenesulfonamide: To a solution of N-(1-{[3,4-bis(methyloxy)phenyl]sulfonyl}-3-piperidinyl)-3,4-bis(methyloxy)benzenesulfonamide (50 mg, 0.1 mmol) and 1-iodoethane (120 uL, 1.5 mmol) in 1 mL acetonitrile was added solid potassium carbonate (69 mg, 0.5 mmol). The suspension was heated at 70° C. for 15 hrs, cooled to room temperature and solids filtered off. The filtrate was subjected to reverse phase HPLC purification. The desired product was afforded as an oil (15 mg, 28% yield). LCMS (M+H=529.2). ¹H NMR (DMSO-d6, 400 MHz) δ: 7.37 (1H, dd), 7.24 (1H, dd), 7.20 (1H, d), 7.14 (1H, d), 7.09 (1H, d), 7.07 (1H, d), 3.82 (6H, s), 3.80 (3H, s), 3.78 (3H, s), 3.70 (1H, m), 3.52 (1H, m), 3.44 (1H, m), 3.11 (2H, m), 2.24 (1H, m), 2.12 (1H, m), 1.64 (1H, m), 1.28-1.47 (3H, m), 1.04 (3 H, t).

### Example 94

### N-[((2S)-1-{[3,4-bis(methyloxy)phenyl]sulfonyl}-2-pyrrolidinyl)methyl]-N-methyl-3,4-bis(methyloxy)benzenesulfonamide

a) Preparation of N-[((2S)-1-{[3,4-bis(methyloxy)phenyl]sulfonyl}-2-pyrrolidinyl)methyl]-3,4-bis(methyloxy)benzenesulfonamide: A solution of [(2S)-2-pyrrolidinylmethyl]amine (50 mg; 0.50 mmol) in 2 mL of DCM was treated with DIEA (194 mg; 1.50 mmol) followed by), 3,4-bis(methyl)oxybenzenesulfonyl chloride (236 mg, 1.00 mmol). The solution was sealed under N2 and allowed to stir at room temperature overnight. The solution was diluted to 10 mL with DCM and washed with 1M NaHSO4, water, and sat'd aq. NaHCO3. The organic phase was dried and concentrated in vacuo to give 199 mg of N-[((2S)-1-{[3,4-bis(methyloxy)phenyl]sulfonyl}-2-pyrrolidinyl)methyl]-3,4-bis(methyloxy)benzenesulfonamide as a white amorphous solid. 99% purity by HPLC. LCMS (M+H=501). ¹H NMR (DMSO-d6, 400 MHz) δ: 7.65 (t, 1H), 7.37 (dd, 1H), 7.31 (d, 1H), 7.28 (dd, 1H), 7.18 (d, 1H), 7.14 (d, 1H), 7.12 (d, 1H), 3.83 (bs, 6H), 3.80 (bs, 6H), 3.47 (m, 1H), 3.25 (m, 1H), 3.01 (m, 2H), 2.68 (m, 1H), 1.68 (m, 2H), 1.39 (m, 2H).

b) Preparation of N-[((2S)-1-{[3,4-bis(methyloxy)phenyl]sulfonyl}-2-pyrrolidinyl)methyl]-N-methyl-3,4-bis(methyloxy)benzenesulfonamide: A solution of N-[((2S)-1-{[3,4-bis(methyloxy)phenyl]sulfonyl}-2-pyrrolidinyl)methyl]-3,4-bis(methyloxy)benzenesulfonamide (42 mg; 0.08 mmol) in 2 mL of CH3CN was treated with K2CO3 (0.1 g; 0.7 mmol) followed by iodomethane (36 mg; 0.25 mmol). The mixture was sealed under N2 and heated at 40° C. overnight. The reaction was diluted with DCM and filtered through Celite. The filtrate was concentrated in vacuo, and the sample was triturated with DCM. The supernatant was removed, filtered, and concentrated in vacuo to give 44 mg of the title compound as white amorphous solid. 100% purity by HPLC. LC/MS (M+H=515). ¹H NMR (DMSO-d6, 400 MHz) δ: 7.41 (dd, 1H), 7.37 (dd, 1H), 7.24 (d, 1H), 7.23 (d, 1H), 7.17 (d, 1H), 7.15 (d, 1H), 3.83 (m, 12H), 3.74 (m, 1H), 3.30 (unresolved from water), 3.02 (m, 4H), 2.72 (s, 3H), 1.78 (m, 2H), 1.48 (m, 2H).

### Example 95

### N-[((2S)-1-{[3,4-bis(methyloxy)phenyl]sulfonyl}-2-pyrrolidinyl)methyl]-N-ethyl-3,4-bis(methyloxy)benzenesulfonamide

This sample was prepared as described in Example 94b except that iodoethane was substituted for iodomethane to give 43 mg of the title compound as amorphous yellow solid. 99% purity by HPLC. LC/MS (M+H=529). ¹H NMR (DMSO-d6, 400 MHz) δ: 7.42 (dd, 1H), 7.39 (dd, 1H), 7.28 (d, 1H), 7.26 (d, 1H), 7.18 (d, 1H), 7.15 (d, 1H), 3.84 (s, 6H), 3.83 (s, 3H), 3.81 (s, 3H), 3.77 (m, 1H), 3.20 (m, 2H), 3.06 (m, 3H), 1.78 (m, 2H), 1.46 (m, 2H), 1.01 (t, 3H).

### Example 96

### N-[((2S)-1-{[3,4-bis(methyloxy)phenyl]sulfonyl}-2-pyrrolidinyl)methyl]-3,4-bis(methyloxy)-N-propylbenzenesulfonamide

This sample was prepared as described in Example 94b except that iodopropane was substituted for iodomethane to give 44 mg of the title compound as amorphous solid. 99% purity by HPLC. LC/MS (M+H=543). ¹H NMR (DMSO-d6, 400 MHz) δ: 7.40 (m, 2H), 7.29 (d, 1H), 7.26 (d, 1H), 7.17 (m, 2H), 3.83 (m, 12H), 3.77 (m, 1H), 3.16 (dd, 1H), 3.05 (m, 3H), 2.92 (m, 1H), 1.78 (m, 2H), 1.45 (m, 2H), 0.79 (t, 3H).

### Example 97

### N-[((2R)-1-{[3,4-bis(methyloxy)phenyl]sulfonyl}-2-pyrrolidinyl)methyl]-N-methyl-3,4-bis(methyloxy)benzenesulfonamide

a) Preparation of N-[((2R)-1-{[3,4-bis(methyloxy)phenyl]sulfonyl}-2-pyrrolidinyl)methyl]-3,4-bis(methyloxy)benzenesulfonamide: Under anhydrous conditions and nitrogen atmosphere, a solution of D-prolinamide (515 mg; 4.51 mmol) in 10 mL of THF was cooled to 0° C. and treated with 13.5 mL 1.0 M lithium aluminum hydride in THF added dropwise. The mixture was stirred at 0° C. for 30 min. then heated at 75° C. overnight. The resulting suspension was cooled in an ice bath and quenched by dropwise addition of saturated aquesou Na2SO4. The quenched mixture was filtered and the filtrate was concentrated in vacuo. The sample was taken up in EtOAc and dried with Na2SO4 then concentrated in vacuo to give slightly yellow oil which was taken up in 10 mL of DCM and treated with DIEA (2.36 mL; 13.5 mmol) followed by 3,4-bis(methyloxy)benzenesulfonyl chloride (2.14 g; 9.03 mmol). The mixture was capped under nitrogen and stirred at room temperature overnight. The solution was diluted to with DCM and washed with 1M NaHSO4, water, and sat'd aq. NaHCO3. The organic phase was dried with Na2SO4 and concentrated in vacuo to give crude product which was purified by column chromatography on silica gel eluting with DCM and EtOAc to give 1.085 g of N-[((2R)-1-{[3,4-bis(methyloxy)phenyl]sulfonyl}-2-pyrrolidinyl)methyl]-3,4-bis(methyloxy)benzenesulfonamide as white amorphous solid. >99% purity by HPLC. 98.6% enantiomeric purity by chiral SFC. LC/MS (M+H=501). ¹H NMR (DMSO-d6, 400 MHz) δ: 7.65 (t, 1H), 7.37 (dd, 1H), 7.31 (d, 1H), 7.28 (dd, 1H), 7.18 (d, 1H), 7.14 (d, 1H), 7.12 (d, 1H), 3.82 (m, 12H), 3.47 (m, 1H), 3.25 (m, 1H), 3.01 (m, 2H), 2.67 (m, 1H), 1.68 (m, 2H), 1.39 (m, 2H).

b) Preparation of N-[((2R)-1-{[3,4-bis(methyloxy)phenyl]sulfonyl}-2-pyrrolidinyl)methyl]-N-methyl-3,4-bis(methyloxy)benzenesulfonamide: A solution of N-[((2R)-1-{[3,4-bis(methyloxy)phenyl]sulfonyl}-2-pyrrolidinyl)methyl]-3,4-bis(methyloxy)benzenesulfonamide (40 mg; 0.08 mmol) in 2 mL of CH3CN was treated with potassium carbonate (>50 mg; >0.36 mmol) followed by iodomethane (15 uL; 0.24 mmol). The mixture was sealed under N2 and heated at 40° C. overnight. The reaction was diluted with DCM and filtered through Celite. The filtrate was concentrated in vacuo, and the sample was triturated with DCM. The supernatant was removed, filtered, and concentrated in vacuo to give 43 mg of the title compound as white amorphous solid. >99% purity by HPLC. LC/MS (M+H=515). ¹H NMR (DMSO-d6, 400 MHz) δ: 7.39 (m, 2H), 7.23 (d, 2H), 7.16 (m, 2H), 3.83 (m, 12H), 3.74 (m, 1H), 3.02 (m, 3H), 2.72 (s, 3H), 1.78 (m, 2H), 1.48 (m, 2H).

### Example 98

### N-[((2R)-1-{[3,4-bis(methyloxy)phenyl]sulfonyl}-2-pyrrolidinyl)methyl]-N-ethyl-3,4-bis(methyloxy)benzenesulfonamide

This sample was prepared as described in Example 97b except that iodoethane was substituted for iodomethane to give 48 mg of the title compound as an amorphous solid. >99% purity by HPLC. LC/MS (M+H=529). ¹H NMR (DMSO-d6, 400 MHz) δ: 7.42 (dd, 1H), 7.39 (dd, 1H), 7.28 (d, 1H), 7.26 (d, 1H), 7.18 (d, 1H), 7.15 (d, 1H), 3.84 (s, 6H), 3.83 (s, 3H), 3.81 (s, 3H), 3.77 (m, 1H), 3.20 (m, 2H), 3.06 (m, 3H), 1.78 (m, 2H), 1.46 (m, 2H), 1.01 (t, 3H).

### Example 99

### N-[((2R)-1-{[3,4-bis(methyloxy)phenyl]sulfonyl}-2-pyrrolidinyl)methyl]-3,4-bis(methyloxy)-N-propylbenzenesulfonamide

This sample was prepared as described in Example 97b except that 1-iodopropane was substituted for iodomethane to give 41 mg of the title compound as an amorphous solid. >99% purity by HPLC. LC/MS (M+H=543). ¹H NMR (DMSO-d6, 400 MHz) δ: 7.40 (m, 2H), 7.27 (m, 1H), 7.17 (m, 2H), 3.83 (m, 12H), 3.78 (m, 1H), 3.30 (signal unresolved from water), 3.16 (m, 1H), 3.05 (m, 3H), 2.92 (m, 1H), 1.77 (m, 2H), 1.45 (m, 4H), 0.79 (t, 3H).

### Example 100

### N-[((2R)-1-{[3,4-bis(methyloxy)phenyl]sulfonyl}-2-pyrrolidinyl)methyl]-N-(1-methylethyl)-3,4-bis(methyloxy)benzenesulfonamide

This sample was prepared as described in Example 97b except that 2-iodopropane was substituted for iodomethane and the product was purified by column chromatography on silical gel eluting with DCM and EtOAc to give 32 mg of the title compound as a yellow crystalline solid. >99% purity by HPLC. LC/MS (M+H=543). ¹H NMR (DMSO-d6, 400 MHz) δ: 7.42 (m, 2H), 7.30 (m, 1H), 7.17 (m, 2H), 3.98 (m, 2H), 3.82 (m, 12H), 3.31 (signal unresolved from water), 3.18 (m, 1H), 3.05 (m, 3H), 1.82 (m, 2H), 1.41 (m, 2H), 0.96 (d, 3H), 0.81 (d, 3H).

### Biological Section

### Human AXOR109/U2OS/Assay

Human osteosarcoma cell line U2OS has been shown to express and couple 7™ receptors efficiently and therefore were selected as the host line. An MRECRE (multiple response element/cAMP response element) directed reporter gene assay was used for compound profiling against AXOR109. Compounds of the present invention were tested in U2OS recombinant cells in an assay similar to the one described below.

U2OS cells were grown in DMEM/F12 without phenol red plus 10% FBS and 2 mM L-Glutamine (Cell Growth Media). AXOR109 and MRECRE-Luciferase BacMam virus reporter constructs were transduced into U2OS cells. In these recombinant cells, bile acids induced AXOR109 mediated elevation of cAMP. Thus, lithocolic acid was used as a positive control.

Tested compounds were serially diluted (usually 3-fold) to yield 11 point concentration response curves. At 18 to 24 hours post-transduction of virus, cells were incubated with the tested compounds at 37° C. for approximately 5 hours. Following incubation, luminescence was measured using a kit such as Steady-Glo® Luciferase Assay System (Promega). Generally, compounds that exhibit a pEC50 of ≧5.0 are preferred.

### Inhibition of LPS-Induced TNF-Alpha in Human Monocytes Assay

Whole blood was collected from donors in heparinized syringes. PBMC's (peripheral blood mononuclear cells) were isolated from the blood by centrifugation in Accuspin System-Histopaque tubes (Sigma). Monocytes were isolated from the PBMC's using the Miltenyi MACS CD14 microbeads. Monocytes were counted and plated in 96 well plates at a density of 40,000 cells/well.

Tested compounds were diluted in PBS to a 10× concentration and then added to the cells so that the final concentration was 1×. The highest drug concentrations used were 30-50 uM. For dose response curves, 8-10 point 1:2 dilutions beginning at the highest concentration were done. The final DMSO concentration was not greater than 0.5%.

Compounds were incubated on the monocytes for 1 hour. LPS (Lipopolysaccarhide) was added at a final concentration of 1 ng/ml to all wells except the no treatment controls. The cells were incubated overnight at 37 C, 5% CO2. The next day, the plate was spun to pellet the cells and the supernatant removed and frozen at −70 C. A human TNFalpha ELISA kit (R&D Systems) was used to measure the amount of TNFalpha in the supernatant. The supernatant was diluted 1:8 in the assay dilution buffer. The ELISA was performed according to the kit instructions. The ELISA plate absorbance was read at 450 nm on a SpectraMax spectrophotometer (Molecular Devices).

A TNF-alpha standard curve was run on each plate. Optical density values for each well were used to back-calculate a value for TNF-alpha from the standard curve. The percent of TNF-alpha inhibition was calculated by comparing the test compound well to wells that received no compound but were stimulated by LPS (100% stimulation of TNF-alpha). Generally, compounds that exhibit a pEC50≧5.0 are preferred.

### Human AXOR109/Melanophore Assay

AXOR109 is a Gs-coupled receptor and therefore increases intracellular cAMP leading to melanosome dispersion. Compounds of the present invention were characterized in a melanophore assay as described in Jayawickreme C K, et al., Current Protocols in Pharmacology (2005) 12.9.1-12.9.16. Briefly, melanophore cells are transfected by electroporation with AXOR109 expressing cDNA. Cells are then plated in 384 well clear bottom plates at 8E3 cells per well in 50 μl of conditioned fibroblast media (CFM). Compounds to be tested are serially diluted 3-fold in DMSO and further diluted 1:100 with melanophore assay buffer (MAB)+10 nM Melatonin to yield 11 point concentration response curves. Following 2 hour incubation with MAB+1% DMSO+10 nM Melatonin, initial transmittance is read on a TwisterII/Spectramax plate reader. Compounds are then added to the cells and incubated for 1 hour, and final transmittance is read as before. Results are compared to vehicle (MAB+1% DMSO+10 nM Melatonin) and to a positive control (MAB+1% DMSO+10 nM Melatonin+200 nM alpha-MSH). Data are calculated for each well as follows: 1-(Transmitance-final/Transmitance-initial). Each well result is then normalized to alpha-MSH, the system max control for Gs/Gq coupled receptors using the following equation: (ObjX−Control1)/(Control2−Control1)*100. The percent system max is used to generate the pEC50 using the equation: y=((Vmax*x̂n)/(K̂n+x̂n))+Y2. Acceptable Z-prime values should be >0.4. Compounds of the present invention have a pEC50≧5.0 in the above described assay. The pEC50 and % max for the compounds of the present invention are summarized in columns 4 and 5 of Table 1 below.

TABLE 1

| Example | | | | |
|---|---|---|---|---|
| No. | Structure | (M + H) | pEC₅₀* | % Max^{†} |
| 1 | 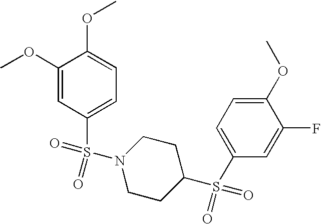 | 475.2 | ++ | ** |
| 2 | 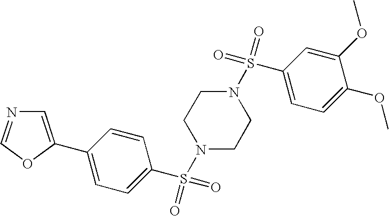 | 494.0 | ++ | ** |
| 3 | 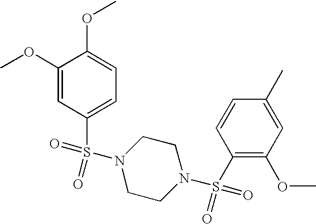 | 471.2 | + | ** |
| 4 | 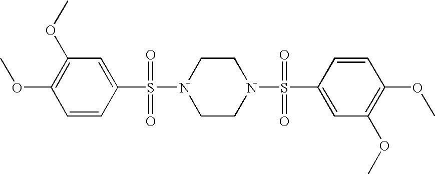 | 487.2 | + | ** |
| 5 | 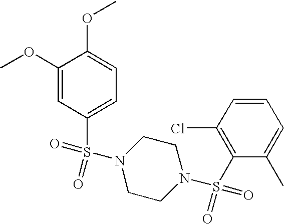 | 475.2 | + | *** |
| 6 | 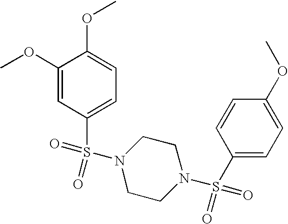 | 457.2 | ++ | * |
| 7 | 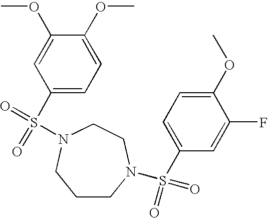 | 489.2 | + | * |
| 8 | 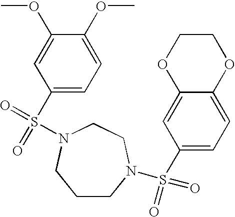 | 499.2 | + | ** |
| 9 | 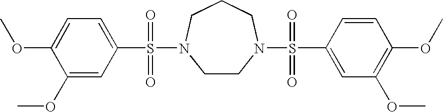 | 501.2 | ++ | ** |
| 10 | 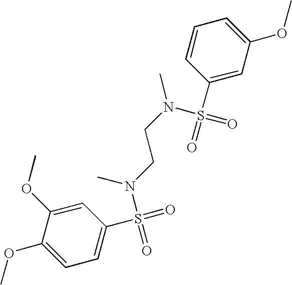 | 459.2 | ++ | *** |
| 11 | 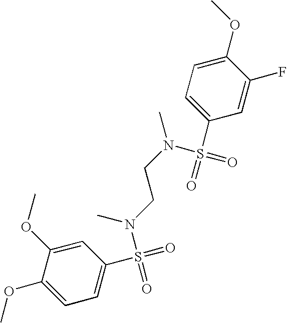 | 477.0 | ++ | * |
| 12 | 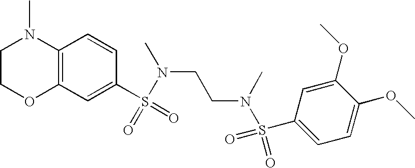 | 500.0 | + | *** |
| 13 | 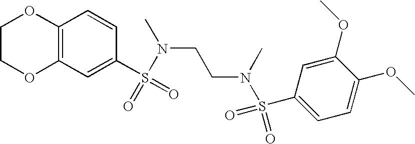 | 487.2 | + | ** |
| 14 | 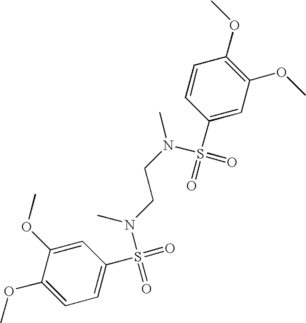 | 489.2 | +++ | ** |
| 15 | 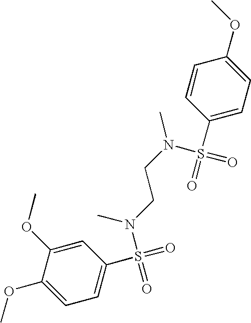 | 459.2 | ++ | *** |
| 16 | 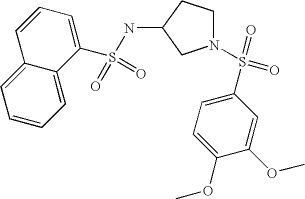 | 478.2 | + | ** |
| 17 | 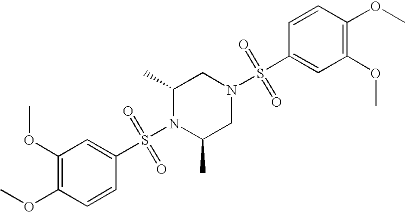 | 515.4 | ++ | * |
| 18 | 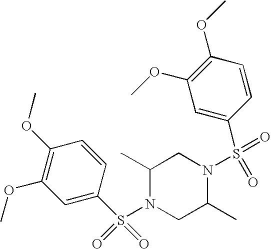 | 515.4 | +++ | ** |
| 19 | 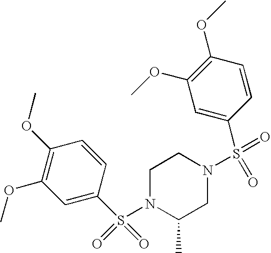 | 501.2 | + | *** |
| 20 | 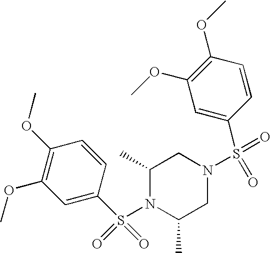 | 515.4 | + | ** |
| 21 | 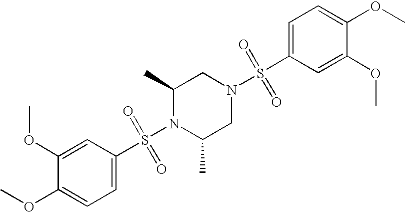 | 515.4 | ++ | ** |
| 22 | 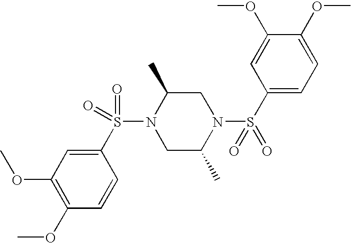 | 515.1 | + | ** |
| 23 | 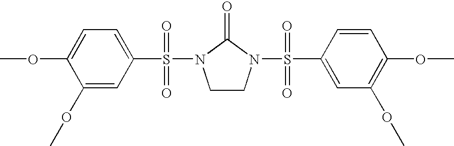 | 487.2 | + | * |
| 24 | 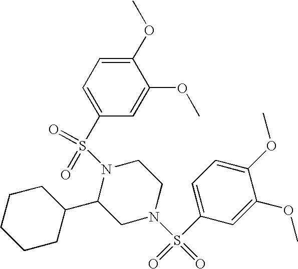 | 569.4 | + | ** |
| 25 | 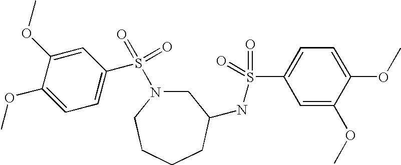 | 515.2 | + | * |
| 26 | 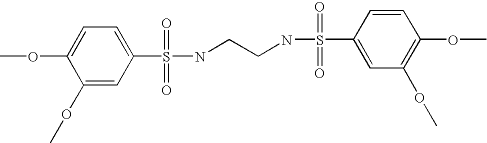 | 461.2 | + | *** |
| 27 | 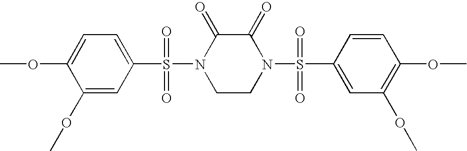 | 515.2 | + | ** |
| 28 | 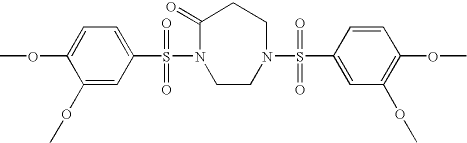 | 515.2 | ++ | ** |
| 29 | 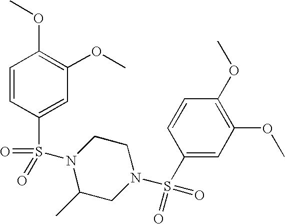 | 501.4 | ++ | ** |
| 30 | 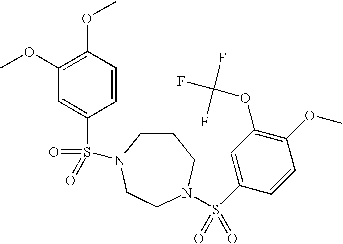 | 555.4 | + | *** |
| 31 | 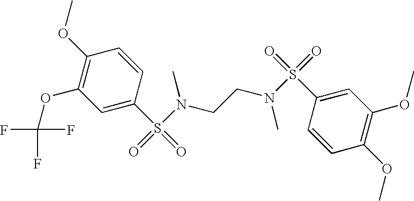 | 543.4 | ++ | * |
| 32 | 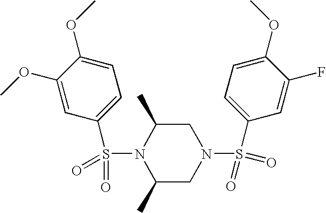 | 503.2 | ++ | ** |
| 33 | 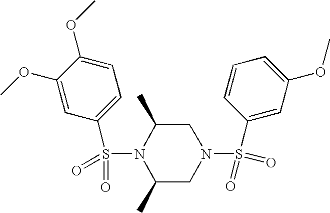 | 485.4 | + | ** |
| 34 | 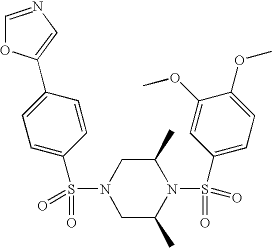 | 522.4 | + | ** |
| 35 | 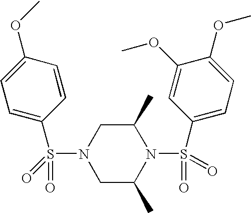 | 485.6 | ++ | ** |
| 36 | 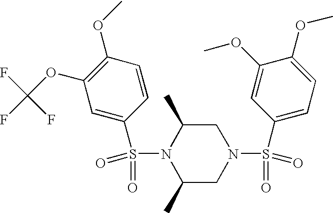 | 569.4 | ++ | ** |
| 37 | 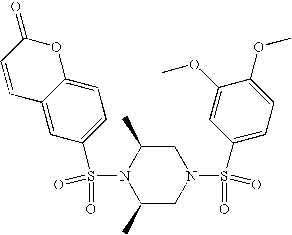 | 523.4 | + | * |
| 38 | 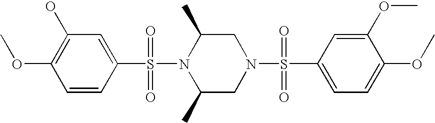 | 501.4 | + | ** |
| 39 | 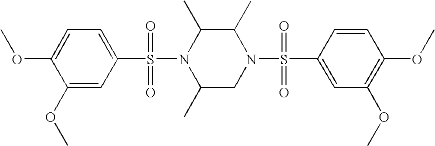 | 547 | + | * |
| 40 | 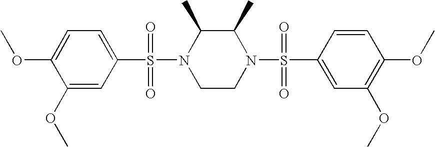 | 515 | ++ | ** |
| 41 | 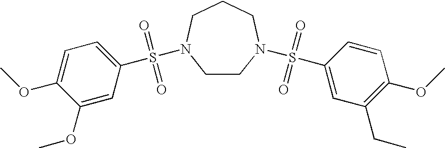 | 499 | ++ | ** |
| 42 | 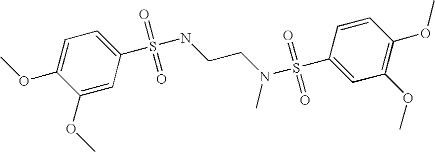 | 475 | + | ** |
| 43 | 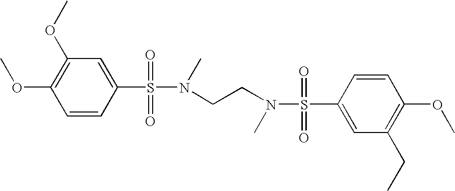 | 487 | ++ | * |
| 44 | 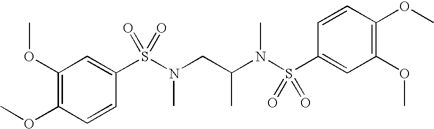 | 503 | ++ | ** |
| 45 | 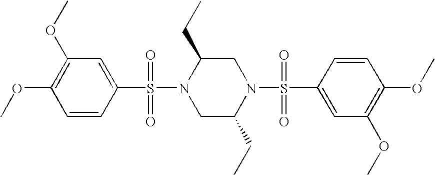 | 543 | + | *** |
| 46 | 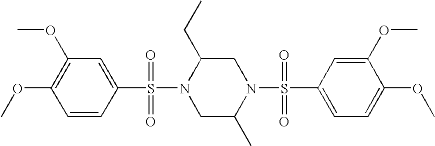 | 529 | + | ** |
| 47 | 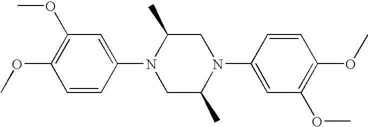 | 515 | +++ | ** |
| 48 | 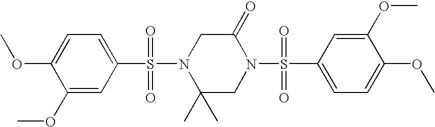 | 529 | + | *** |
| 49 | 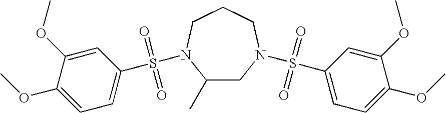 | 515 | + | ** |
| 50 | 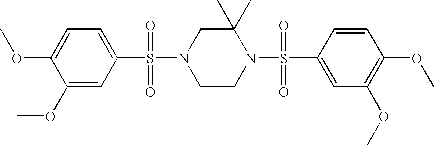 | 515 | ++ | *** |
| 51 | 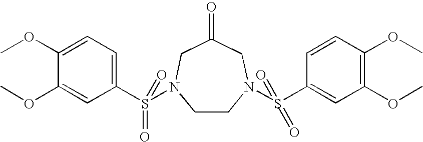 | 515 | + | *** |
| 52 | 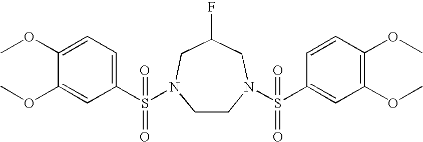 | 519 | ++ | *** |
| 53 | 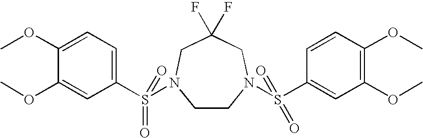 | 537 | ++ | ** |
| 54 | 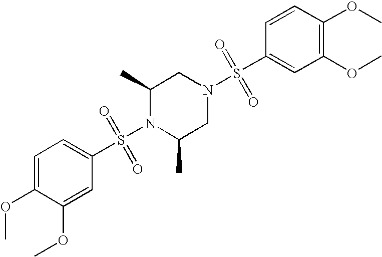 | 515 | ++ | * |
| 55 | 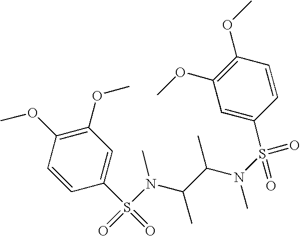 | 517 | ++ | ** |
| 56 | 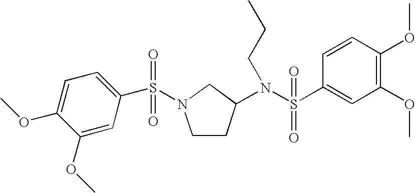 | 529.1 | ++ | * |
| 57 | 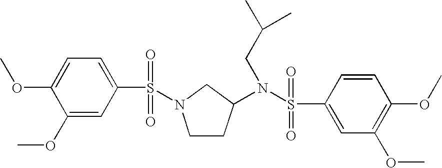 | 543.2 | + | ** |
| 58 | 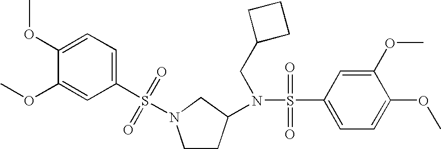 | 555.1 | + | *** |
| 59 | 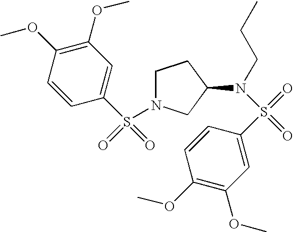 | 529 | ++ | *** |
| 60 | 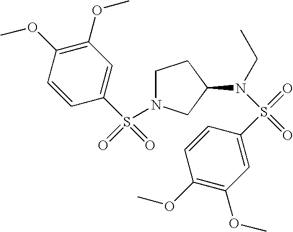 | 515.2 | +++ | *** |
| 61 | 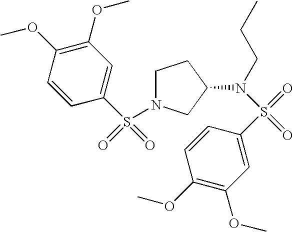 | 529 | + | *** |
| 62 | 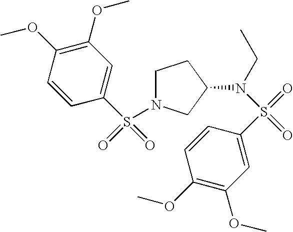 | 515.2 | + | *** |
| 63 | 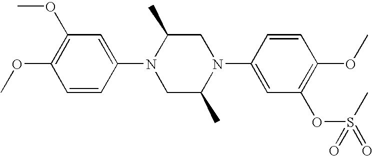 | 579 | + | *** |
| 64 | 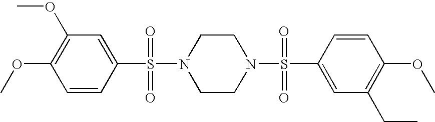 | 485 | ++ | *** |
| 65 | 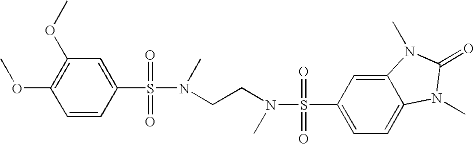 | 513 | + | ** |
| 66 | 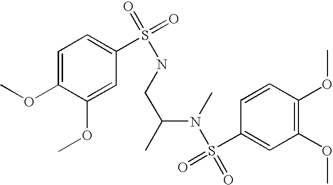 | 489 | + | ** |
| 67 | 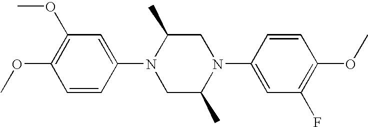 | 503 | ++ | *** |
| 68 | 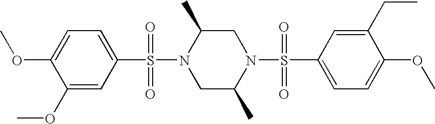 | 513 | ++ | *** |
| 69 | 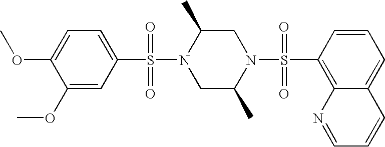 | 506 | + | *** |
| 70 | 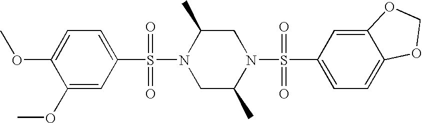 | 499 | ++ | **** |
| 71 | 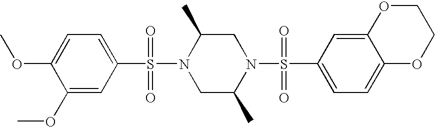 | 513 | ++ | *** |
| 72 | 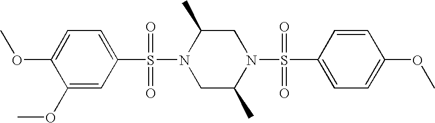 | 485 | ++ | **** |
| 73 | 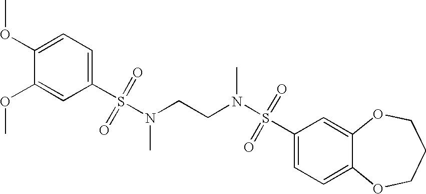 | 501 | + | *** |
| 74 | 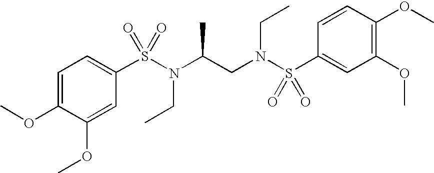 | 531 | ++ | *** |
| 75 | 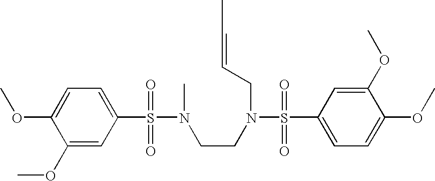 | 529 | ++ | *** |
| 76 | 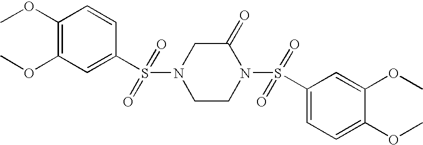 | 501 | + | ** |
| 77 | 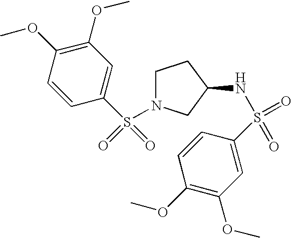 | 487 | + | *** |
| 78 | 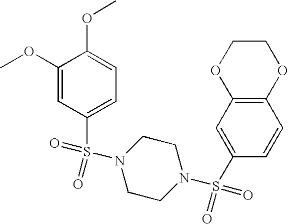 | 485 | + | ** |
| 79 | 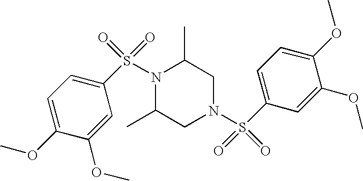 | 515 | +++ | **** |
| 80 | 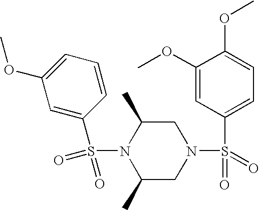 | 485 | + | ** |
| 81 | 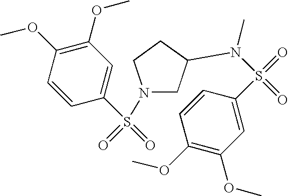 | 501 | ++ | * |
| 82 | 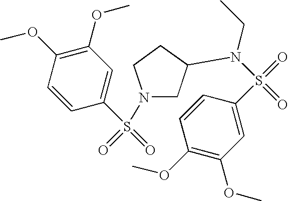 | 515 | ++ | ** |
| 83 | 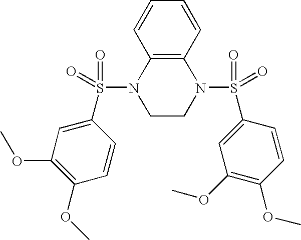 | 535 | ++ | ** |
| 84 | 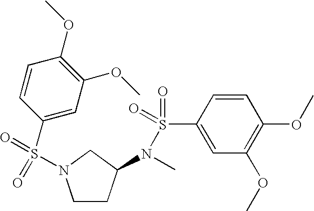 | 501 | + | ** |
| 85 | 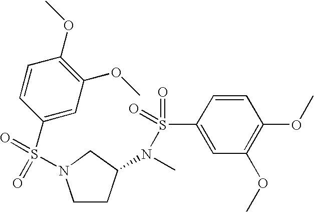 | 501 | ++ | ** |
| 86 | 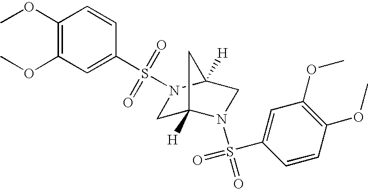 | 499 | + | *** |
| 87 | 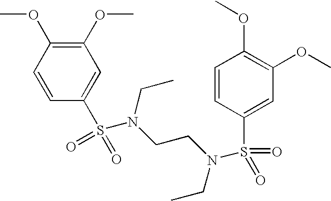 | 517 | ++ | ** |
| 88 | 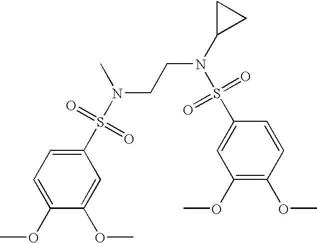 | 515 | ++ | *** |
| 89 | 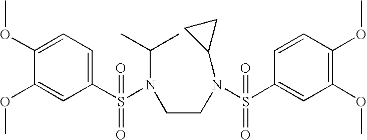 | 543 | ++ | *** |
| 90 | 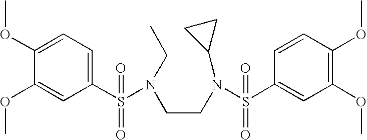 | 529 | ++ | ** |
| 91 | 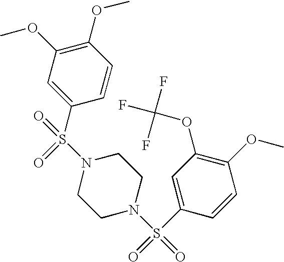 | 541 | ++ | *** |
| 92 | 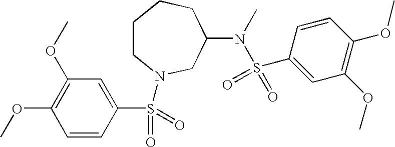 | 529 | + | ** |
| 93 | 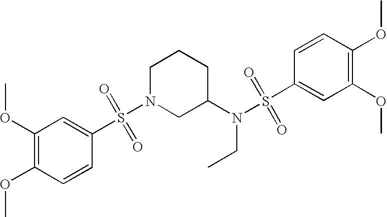 | 529 | + | ** |
| 94 | 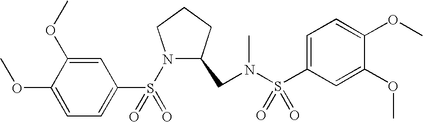 | 515 | + | *** |
| 95 | 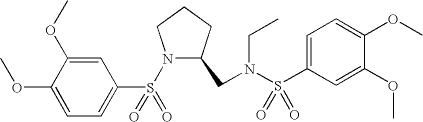 | 529 | ++ | *** |
| 96 | 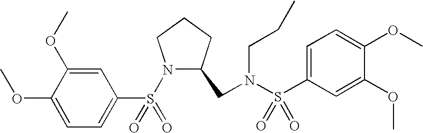 | 543 | ++ | *** |
| 97 | 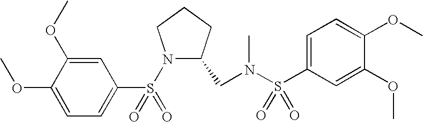 | 515 | +++ | *** |
| 98 | 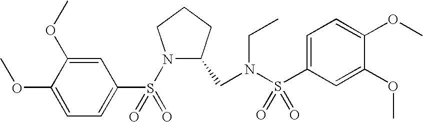 | 529 | +++ | *** |
| 99 | 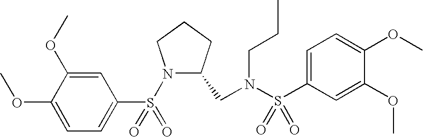 | 543 | +++ | *** |
| 100 | 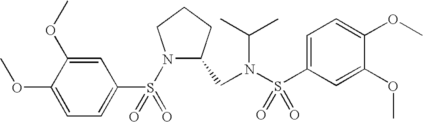 | 543 | ++ | **** |

*pEC50 from melanophore assay as described above. “+” = 5.0 to 5.9, “++” = 6.0 to 6.9, “+++” = >6.9.
^{†}% Max from melanophore assay as described above. “*” = 0% to 50%, “**” = 51% to 75%, “***” = 76% to 100%, “****” = >100%.

### In Vivo Studies

### Effect of a Representative Compound on GLP-1 Secretion in CD Rats.

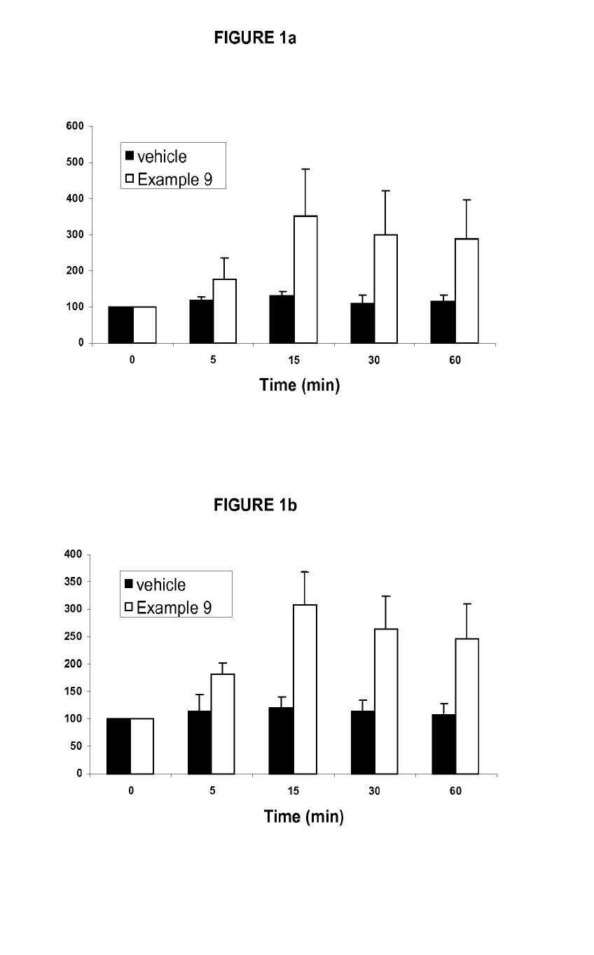

Twelve ten week old (six per group) anesthetized normal CD rats were dosed with vehicle (0.5% HPMC/0.1% Tween80) or 2.5 mg Example 9 through intracolonic injection at 0 time point after a control blood sample was collected. Blood samples were collected at 5, 15, 30 and 60 minutes after dosing. Plasma active, and plsdms total GLP-1 were measured (ELISA kits, LINCO), and values were converted to % of controls. Animals receiving Example 9 demonstrated a higher level of active and total GLP-1 when compared to controls. The results are listed in Tables 2a and 2b, and graphically represented in FIGS. 1a and 1b, respectively.

TABLE 2a
Portal Vein Active GLP-1 Levels (% of Controls)

| Time (min) | vehicle | Example 9 |
|---|---|---|
| 0 | 100.00 | 100.00 |
| 5 | 117.82 | 175.27 |
| 15 | 130.17 | 352.50 |
| 30 | 110.57 | 298.49 |
| 60 | 114.83 | 286.95 |

TABLE 2b
Portal Vein Total GLP-1 Levels (% of Controls)

| Time (min) | vehicle | Example 9 |
|---|---|---|
| 0 | 100.00 | 100.00 |
| 5 | 114.44 | 182.84 |
| 15 | 120.09 | 308.38 |
| 30 | 114.21 | 263.34 |
| 60 | 107.73 | 246.00 |

### Effect of a Representative Compound on Glucose, Insulin, and GLP-1 Secretion in GK Rats.

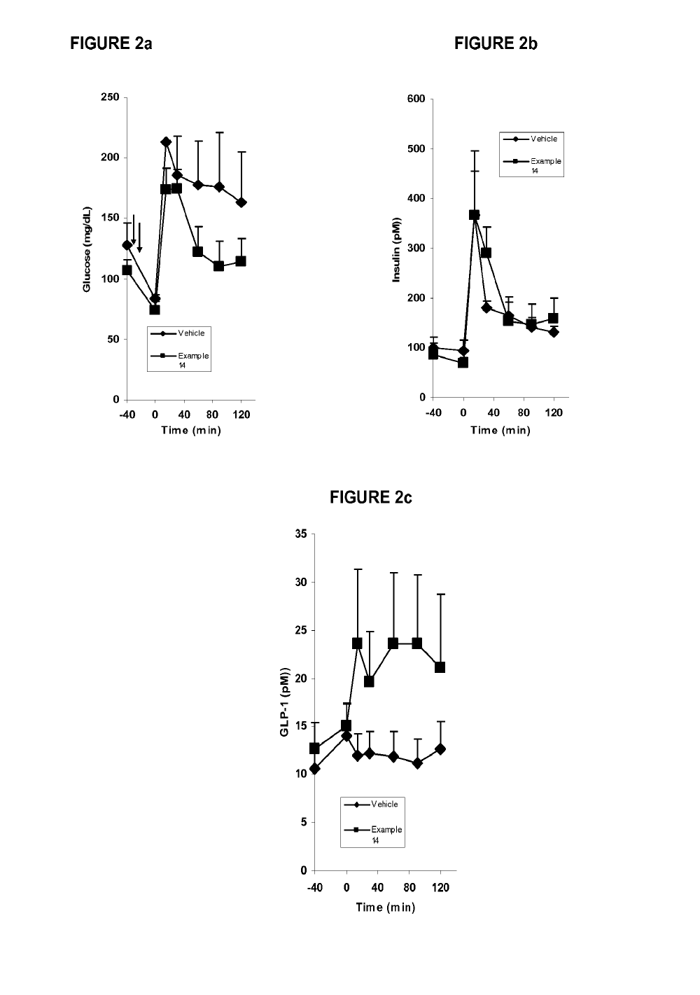

Eight week old conscious GK rats (four per group) were pretreated with a DPPIV inhibitor immediately after control blood sampling at −40 min (black arrow), and dosed with vehicle (0.5% HPMC/0.1% Tween80) or 5 mg/kg Example 14 through intracolonic injection at −30 time point (blue arrow). Glucose (1 g/kg) was given orally at 0 min. Blood samples were collected at −40, 0, 5, 15, 30, 60, 90 and 120 minutes after dosing. Whole blood glucose, plasma insulin, and active GLP-1 were measured (ELISA kits, LINCO). Animals receiving Example 14 demonstrated a higher level of active GLP-1 and insulin, with improved glucose disposals, when compared to controls. The results are listed in Tables 3a, 3b, and 3c, and graphically represented in FIGS. 2a, 2b, and 2c, respectively.

TABLE 3a
Glucose changes

| Time (min) | Vehicle | Example 14 |
|---|---|---|
| −40 | 127.75 | 106.75 |
| 0 | 83.75 | 74.25 |
| 15 | 212.67 | 173.75 |
| 30 | 185.75 | 174.75 |
| 60 | 178.00 | 122.50 |
| 90 | 176.00 | 110.00 |
| 120 | 163.50 | 114.50 |

TABLE 3b
Insulin changes

| Time (min) | Vehicle | Example 14 |
|---|---|---|
| −40 | 100.58 | 85.82 |
| 0 | 94.43 | 68.06 |
| 15 | 367.41 | 366.79 |
| 30 | 180.24 | 289.23 |
| 60 | 164.06 | 152.33 |
| 90 | 140.28 | 146.47 |
| 120 | 131.61 | 158.31 |

TABLE 3c
GLP-1 changes

| Time (min) | Vehicle | Example 14 |
|---|---|---|
| −40 | 10.64 | 12.65 |
| 0 | 13.97 | 15.08 |
| 15 | 11.92 | 23.63 |
| 30 | 12.22 | 19.58 |
| 60 | 11.88 | 23.63 |
| 90 | 11.13 | 23.63 |
| 120 | 12.71 | 21.15 |

Effect of a Representative Compound on Prevention of Hyperglycemia, Increased Insulin Levels, and Increased Glucagon Levels, after Chronic Dosing in GK Rats.

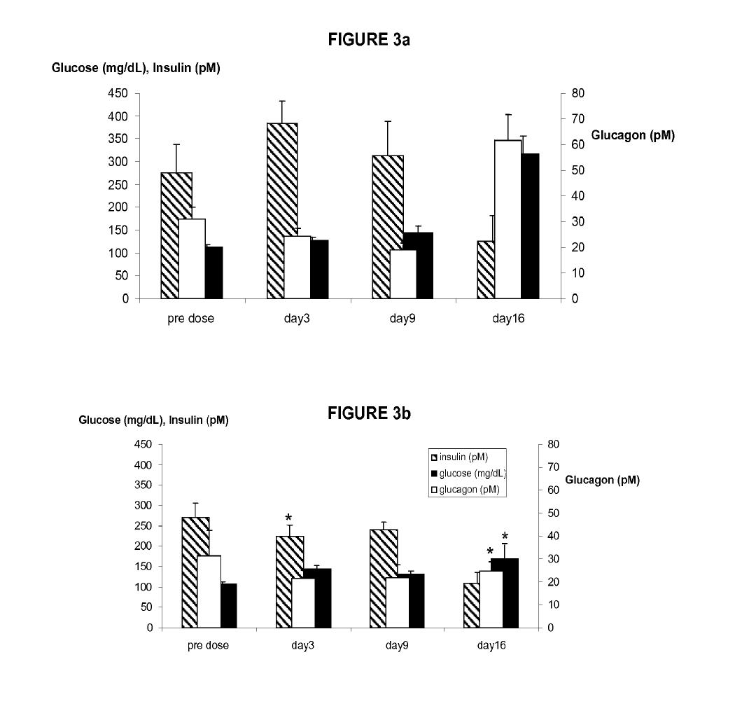

A sixteen day chronic study in conscious GK rats (5 animals per group) with intracolonic dosing of Example 9 (0.3 mg/kg, QD) showed that no hyperglycemia had developed when compared with rats receiving vehicle (0.5% HPMC/0.1% Tween80). Increases in insulin at day 3 and day 9 were observed in the vehicle group prior to observing hyperglycemia, and these rats also showed high glucagon levels at day 16. However, the rats treated with Example 9 showed normal insulin levels and no increase in glucagons at the equivalent time points. The results are listed in Tables 4a and 4b, and graphically represented in FIGS. 3a and 3b, respectively.

TABLE 4a

| | pre | | | | | | | |
|---|---|---|---|---|---|---|---|---|
| Vehicle | dose | SE | day 3 | SE | day 9 | SE | day 16 | SE |
| insulin | 275.52 | 62.65 | 383.84 | 49.88 | 313.78 | 73.54 | 126.56 | 56.06 |
| (pM) | | | | | | | | |
| glucose | 112.20 | 5.36 | 126.60 | 7.61 | 145.00 | 14.86 | 316.50 | 39.08 |
| (mg/dL) | | | | | | | | |
| glucagon | 30.84 | 4.97 | 24.23 | 3.18 | 18.86 | 2.73 | 61.83 | 9.91 |
| (pM) | | | | | | | | |

TABLE 4b

| Example | pre | | | | | | | |
|---|---|---|---|---|---|---|---|---|
| 9 | dose | SE | day 3 | SE | day 9 | SE | day 16 | SE |
| insulin | 269.67 | 35.50 | 223.24 | 28.15 | 240.29 | 19.00 | 108.09 | 27.38 |
| (pM) | | | | | | | | |
| glucose | 107.17 | 5.06 | 144.20 | 6.96 | 131.00 | 7.70 | 169.00 | 37.45 |
| (mg/dL) | | | | | | | | |
| glucagon | 31.28 | 10.96 | 21.31 | 3.72 | 21.67 | 5.57 | 24.67 | 4.13 |
| (pM) | | | | | | | | |

### Effect of a Representative Compound on Glucose Tolerance in GK Rats.

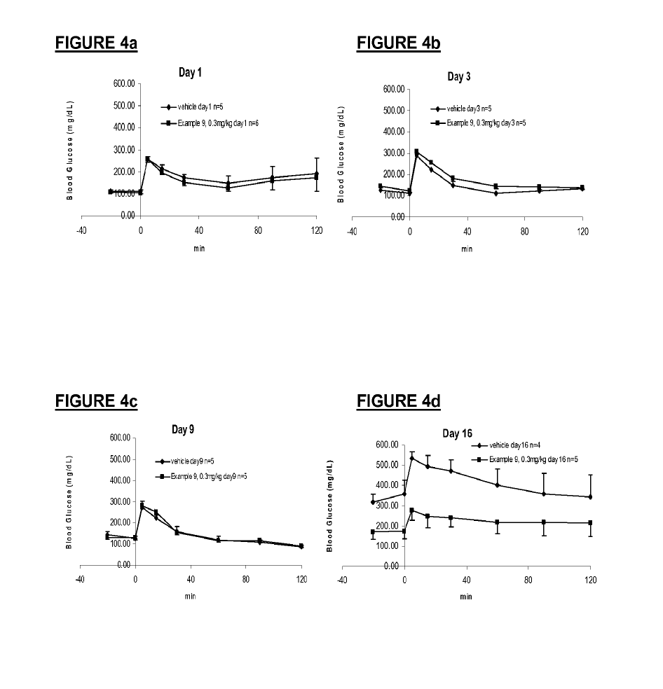

In the same chronic study in GK rats as in Tables 4a and 4b, an intravenous glucose tolerance test was performed on days 1, 3, 9, and 16. On day 16, hyperglycemia had developed in rats receiving vehicle, but not in the rats treated with Example 9. Glucose excretion during the oral glucose tolerance test was less in the treated group as compared to the vehicle group. These results are listed in Tables 5a, 5b, 5c, and 5d, and graphically represented in FIGS. 4a, 4b, 4c, and 4d, respectively.

TABLE 5a

| | vehicle | | Example 9, | |
|---|---|---|---|---|
| Time | day 1 | | 0.3 mg/kg day 1 | |
| (min) | n = 5 | SE | n = 6 | SE |
| −20.00 | 112.20 | 5.36 | 107.17 | 5.06 |
| 0.00 | 110.80 | 8.66 | 103.67 | 5.55 |
| 5.00 | 257.00 | 11.32 | 257.17 | 13.10 |
| 15.00 | 215.00 | 15.97 | 195.67 | 7.89 |
| 30.00 | 172.40 | 14.79 | 149.50 | 12.01 |
| 60.00 | 148.80 | 30.93 | 126.83 | 15.66 |
| 90.00 | 174.20 | 50.88 | 158.00 | 42.05 |
| 120.00 | 190.20 | 70.75 | 173.50 | 61.84 |

TABLE 5b

| | vehicle | | Example 9, | |
|---|---|---|---|---|
| Time | day 3 | | 0.3 mg/kg day 3 | |
| (min) | n = 5 | SE | n = 5 | SE |
| −20.00 | 126.60 | 7.61 | 144.20 | 6.96 |
| 0.00 | 110.00 | 6.03 | 123.00 | 9.64 |
| 5.00 | 289.00 | 12.32 | 307.40 | 11.58 |
| 15.00 | 220.80 | 11.32 | 254.80 | 6.95 |
| 30.00 | 147.40 | 18.34 | 182.20 | 16.40 |
| 60.00 | 111.20 | 4.61 | 143.60 | 8.68 |
| 90.00 | 122.20 | 5.15 | 140.60 | 11.69 |
| 120.00 | 133.60 | 11.47 | 137.60 | 9.18 |

TABLE 5c

| | vehicle | | Example 9, | |
|---|---|---|---|---|
| Time | day 9 | | 0.3 mg/kg day 9 | |
| (min) | n = 5 | SE | n = 5 | SE |
| −20.00 | 145.00 | 14.86 | 131.00 | 7.70 |
| 0.00 | 127.20 | 9.89 | 129.00 | 9.65 |
| 5.00 | 273.60 | 26.64 | 278.80 | 12.98 |
| 15.00 | 221.20 | 20.92 | 252.40 | 13.83 |
| 30.00 | 158.80 | 24.59 | 154.80 | 10.56 |
| 60.00 | 119.75 | 17.09 | 116.20 | 8.78 |
| 90.00 | 109.50 | 8.90 | 115.00 | 9.15 |
| 120.00 | 84.67 | 8.74 | 88.60 | 7.62 |

TABLE 5d

| | vehicle | | Example 9, | |
|---|---|---|---|---|
| Time | day 16 | | 0.3 mg/kg day 16 | |
| (min) | n = 4 | SE | n = 5 | SE |
| −20.00 | 316.50 | 39.08 | 169.00 | 37.45 |
| 0.00 | 356.75 | 70.58 | 174.20 | 39.43 |
| 5.00 | 535.50 | 32.53 | 275.20 | 47.67 |
| 15.00 | 493.25 | 53.76 | 245.80 | 53.64 |
| 30.00 | 469.75 | 58.29 | 238.80 | 42.78 |
| 60.00 | 402.25 | 81.28 | 215.40 | 55.06 |
| 90.00 | 356.00 | 102.43 | 217.60 | 68.39 |
| 120.00 | 340.50 | 112.36 | 212.60 | 63.88 |

What is claimed is:

1: A pharmaceutical composition comprising a compound selected from
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1,4-bis{[3,4-bis(methyloxy)phenyl]sulfonyl}piperazine;
1-{[3,4-bis(methyloxy)phenyl]sulfonyl}-4-[(2-chloro-6-methylphenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
1,4-bis{[3,4-bis(methyloxy)phenyl]sulfonyl}hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-b is(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1,3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutylmethyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;
or a salt or solvate thereof.

2: A method of treating conditions or disorders in a mammal, wherein said condition or disorder is affected by AXOR 109, comprising administering a pharmaceutical composition as claimed in claim 1.

3: A method of treating conditions or disorders in a mammal, wherein said condition or disorder is affected by GLP-1, comprising administering a pharmaceutical composition as claimed in claim 1.

4: The method of claim 2, wherein said condition or disorder is one or more of type I diabetes, type II diabetes, obesity, appetite control, satiety, glucose intolerance, insulin resistance, metabolic syndrome, hyperlipidemia, hypercholesterolemia, atherosclerosis, inflammation, neurodegenerative diseases, alzheimers, stress disorders, and cerebrovascular conditions.

5: The method of claim 2, wherein said condition or disorder is one or more of type I diabetes, type II diabetes, glucose intolerance, insulin resistance, and metabolic syndrome.

6: The method of claim 2, wherein said administering is by a targeted drug delivery system.

7: The method of claim 6 wherein said targeted drug delivery system is a colon targeted drug delivery system.

8-14. (canceled)

15: A compound selected from:
1-{[3,4-bis(methyloxy)phenyl]sulfonyl}-4-{[3-fluoro-4-(methyloxy)phenyl]sulfonyl}piperazine;
1-{[3,4-bis(methyloxy)phenyl]sulfonyl}-4-{[4-(1,3-oxazol-5-yl)phenyl]sulfonyl}piperazine;
1-{[3,4-bis(methyloxy)phenyl]sulfonyl}-4-{[4-methyl-2-(methyloxy)phenyl]sulfonyl}piperazine;
1-{[3,4-bis(methyloxy)phenyl]sulfonyl}-4-[(2-chloro-6-methylphenyl)sulfonyl]piperazine;
1-{[3,4-bis(methyloxy)phenyl]sulfonyl}-4-{[4-(methyloxy)phenyl]sulfonyl}piperazine;
1-{[3,4-bis(methyloxy)phenyl]sulfonyl}-4-{[3-fluoro-4-(methyloxy)phenyl]sulfonyl}hexahydro-1H-1,4-diazepine;
1-{[3,4-bis(methyloxy)phenyl]sulfonyl}-4-(2,3-dihydro-1,4-benzodioxin-6-ylsulfonyl)hexahydro-1H-1,4-diazepine;
N-methyl-N-[2-(methyl{[3-(methyloxy)phenyl]sulfonyl}amino)ethyl]-3,4-bis(methyloxy)benzenesulfonamide;
N-{2-[{[3,4-bis(methyloxy)phenyl]sulfonyl}(methyl)amino]ethyl}-3-fluoro-N-methyl-4-(methyloxy)benzenesulfonamide;
N-{2-[{[3,4-bis(methyloxy)phenyl]sulfonyl}(methyl)amino]ethyl}-N,4-dimethyl-3,4-dihydro-2H-1,4-benzoxazine-7-sulfonamide;
N-{2-[{[3,4-bis(methyloxy)phenyl]sulfonyl}(methyl)amino]ethyl}-N-methyl-2,3-dihydro-1,4-benzodioxin-6-sulfonamide;
N,N′-1,2-ethanediylbis[N-methyl-3,4-bis(methyloxy)benzenesulfonamide];
N-methyl-N-[2-(methyl{[4-(methyloxy)phenyl]sulfonyl}amino)ethyl]-3,4-bis(methyloxy)benzenesulfonamide;
N-(1-{[3,4-bis(methyloxy)phenyl]sulfonyl}-3-pyrrolidinyl)-8-quinolinesulfonamide;
(2R,6R)-1,4-bis{[3,4-bis(methyloxy)phenyl]sulfonyl}-2,6-dimethylpiperazine;
1,4-bis{[3,4-bis(methyloxy)phenyl]sulfonyl}-2,5-dimethylpiperazine;
(2S)-1,4-bis{[3,4-bis(methyloxy)phenyl]sulfonyl}-2-methylpiperazine;
1,4-bis{[3,4-bis(methyloxy)phenyl]sulfonyl}-2,6-cis-dimethylpiperazine;
(2S,6S)-1,4-bis{[3,4-bis(methyloxy)phenyl]sulfonyl}-2,6-dimethylpiperazine;
trans-1,4-bis{[3,4-bis(methyloxy)phenyl]sulfonyl}-2,5-dimethylpiperazine;
1, 3-bis{[3,4-bis(methyloxy)phenyl]sulfonyl}-2-imidazolidinone;
1,4-bis{[3,4-bis(methyloxy)phenyl]sulfonyl}-2-cyclohexylpiperazine;
N-(1-{[3,4-bis(methyloxy)phenyl]sulfonyl}hexahydro-1H-azepin-3-yl)-3,4-bis(methyloxy)benzenesulfonamide;
N-[2-({[3,4-bis(methyloxy)phenyl]sulfonyl}amino)ethyl]-3-methyl-4-(methyloxy)benzenesulfonamide;
1,4-bis{[3,4-bis(methyloxy)phenyl]sulfonyl}-2,3-piperazinedione;
1,4-bis{[3,4-bis(methyloxy)phenyl]sulfonyl}-2,3-piperazinedione;
1,4-bis{[3,4-bis(methyloxy)phenyl]sulfonyl}-2-methylpiperazine;
1-{[3,4-bis(methyloxy)phenyl]sulfonyl}-4-({4-(methyloxy)-3-[(trifluoromethyl)oxy]phenyl}sulfonyl)hexahydro-1H-1,4-diazepine;
N-methyl-N-{2-[methyl({4-(methyloxy)-3 [(trifluoromethyl)oxy]phenyl}sulfonyl)amino]ethyl}-3,4-bis(methyloxy)benzenesulfonamide;
1-{[3,4-bis(methyloxy)phenyl]sulfonyl}-4-{[3-fluoro-4-(methyloxy)phenyl]sulfonyl}-2,6-cis-dimethylpiperazine;
1-{[3,4-bis(methyloxy)phenyl]sulfonyl}-2,6-cis-dimethyl-4-{[3-(methyloxy)phenyl]sulfonyl}piperazine; 1-{[3,4-bis(methyloxy)phenyl]sulfonyl}-2,6-cis-dimethyl-4-{[4-(1,3-oxazol-5-yl)phenyl]sulfonyl}piperazine; 1-{[3,4-bis(methyloxy)phenyl]sulfonyl}-2,6-cis-dimethyl-4-{[4-(methyloxy)phenyl]sulfonyl}piperazine; 4-{[3,4-bis(methyloxy)phenyl]sulfonyl}-2,6-cis-dimethyl-1-({4-(methyloxy)-3-[(trifluoromethyl)oxy]phenyl}sulfonyl)piperazine;
6-[(4-{[3,4-bis(methyloxy)phenyl]sulfonyl}-2,6-cis-dimethyl-1-piperazinyl)sulfonyl]-2H-chromen-2-one;
5-[(4-{[3,4-bis(methyloxy)phenyl]sulfonyl}-2,6-cis-dimethyl-1-piperazinyl)sulfonyl]-2-(methyloxy)phenol;
1,4-bis{[3,4-bis(methyloxy)phenyl]sulfonyl}-2,3,5-trimethylpiperazine;
1,4-bis{[3,4-bis(methyloxy)phenyl]sulfonyl}-2,3-dimethylpiperazine;
1-{[3,4-bis(methyloxy)phenyl]sulfonyl}-4-{[3-ethyl-4-(methyloxy)phenyl]sulfonyl}hexahydro-1H-1,4-diazepine;
N-[2-({[3,4-bis(methyloxy)phenyl]sulfonyl}amino)ethyl]-N-methyl-3,4-bis(methyloxy)benzenesulfonamide;
N-{2-[{[3,4-bis(methyloxy)phenyl]sulfonyl}(methyl)amino]ethyl}-N-methyl-3-ethyl-4-(methyloxy)benzenesulfonamide;
N,N′-1,2-propanediylbis[N-methyl-3,4-bis(methyloxy)benzenesulfonamide];
(2R,5S)-1,4-bis{[3,4-bis(methyloxy)phenyl]sulfonyl}-2,5-diethylpiperazine;
1,4-bis{[3,4-bis(methyloxy)phenyl]sulfonyl}-2-ethyl-5-methylpiperazine;
(2S,5S)-1,4-bis{[3,4-bis(methyloxy)phenyl]sulfonyl}-2,5-dimethylpiperazine;
1,4-bis{[3,4-bis(methyloxy)phenyl]sulfonyl}-5,5-dimethyl-2-piperazinone;
1,4-bis{[3,4-bis(methyloxy)phenyl]sulfonyl}-2-methylhexahydro-1H-1,4-diazepine;
1,4-bis{[3,4-bis(methyloxy)phenyl]sulfonyl}-2,2-dimethylpiperazine;
1,4-bis{[3,4-bis(methyloxy)phenyl]sulfonyl}hexahydro-6H-1,4-diazepin-6-one;
1,4-bis{[3,4-bis(methyloxy)phenyl]sulfonyl}-6-fluorohexahydro-1H-1,4-diazepine;
1,4-bis{[3,4-bis(methyloxy)phenyl]sulfonyl}-6,6-difluorohexahydro-1H-1,4-diazepine;
(2R,6S)-1,4-bis{[3,4-bis(methyloxy)phenyl]sulfonyl}-2,6-dimethylpiperazine;
N,N′-2,3-butanediylbis[N-methyl-3,4-bis(methyloxy)benzenesulfonamide];
N-(1-{[3,4-bis(methyloxy)phenyl]sulfonyl}-3-pyrrolidinyl)-3,4-bis(methyloxy)-N-propylbenzenesulfonamide;
N-(1-{[3,4-bis(methyloxy)phenyl]sulfonyl}-3-pyrrolidinyl)-3,4-bis(methyloxy)-N-(2-methylpropyl)benzenesulfonamide;
N-(1-{[3,4-bis(methyloxy)phenyl]sulfonyl}-3-pyrrolidinyl)-N-(cyclobutylmethyl)-3,4-bis(methyloxy)benzenesulfonamide;
N-((3R)-1-{[3,4-bis(methyloxy)phenyl]sulfonyl}-3-pyrrolidinyl)-3,4-bis(methyloxy)-N-propylbenzenesulfonamide; N-((3R)-1-{[3,4-bis(methyloxy)phenyl]sulfonyl}-3-pyrrolidinyl)-N-ethyl-3,4-bis(methyloxy)benzenesulfonamide;
N-((3S)-1-{[3,4-bis(methyloxy)phenyl]sulfonyl}-3-pyrrolidinyl)-3,4-bis(methyloxy)-N-propylbenzenesulfonamide;
N-((3S)-1-{[3,4-bis(methyloxy)phenyl]sulfonyl}-3-pyrrolidinyl)-N-ethyl-3,4-bis(methyloxy)benzenesulfonamide;
4-[((2S,5S)-4-{[3,4-bis(methyloxy)phenyl]sulfonyl}-2,5-dimethyl-1-piperazinyl)sulfonyl]-2-(methyloxy)phenyl methanesulfonate;
1-{[3,4-bis(methyloxy)phenyl]sulfonyl}-4-{[3-ethyl-4-(methyloxy)phenyl]sulfonyl}piperazine;
N-{2-[{[3,4-bis(methyloxy)phenyl]sulfonyl}(methyl)amino]ethyl}-N,1,3-trimethyl-2-oxo-2,3-dihydro-1H-benzimidazole-5-sulfonamide;
N-[2-({[3,4-bis(methyloxy)phenyl]sulfonyl}amino)-1-methylethyl]-N-methyl-3,4-bis(methyloxy)benzenesulfonamide;
(2S,5S)-1-{[3,4-bis(methyloxy)phenyl]sulfonyl}-4-{[3-fluoro-4-(methyloxy)phenyl]sulfonyl}-2,5-dimethylpiperazine;
(2S,5S)-1-{[3,4-bis(methyloxy)phenyl]sulfonyl}-4-{[3-ethyl-4-(methyloxy)phenyl]sulfonyl}-2,5-dimethylpiperazine;
8-[((2S,5S)-4-{[3,4-bis(methyloxy)phenyl]sulfonyl}-2,5-dimethyl-1-piperazinyl)sulfonyl]quinoline;
(2S,5S)-1-(1,3-benzodioxol-5-ylsulfonyl)-4-{[3,4-bis(methyloxy)phenyl]sulfonyl}-2,5-dimethylpiperazine;
(2S,5S)-1-{[3,4-bis(methyloxy)phenyl]sulfonyl}-4-(2,3-dihydro-1,4-benzodioxin-6-ylsulfonyl)-2,5-dimethylpiperazine;
(2S,5S)-1-{[3,4-bis(methyloxy)phenyl]sulfonyl}-2,5-dimethyl-4-{[4-(methyloxy)phenyl]sulfonyl}piperazine;
N-{2-[{[3,4-bis(methyloxy)phenyl]sulfonyl}(methyl)amino]ethyl}-N-methyl-3,4-dihydro-2H-1,5-benzodioxepin-7-sulfonamide;
N,N′-(2S)-1,2-propanediylbis[N-ethyl-3,4-bis(methyloxy)benzenesulfonamide];
N-{2-[{[3,4-bis(methyloxy)phenyl]sulfonyl}(2-buten-1-yl)amino]ethyl}-N-methyl-3,4-bis(methyloxy)benzenesulfonamide;
or a salt or solvate thereof.

16: A method of treating conditions or disorders in a mammal, wherein said condition or disorder is affected by AXOR 109, comprising administering a compound as claimed in claim 15.

17: A method of treating conditions or disorders in a mammal, wherein said condition or disorder is affected by GLP-1, comprising administering a compound as claimed in claim 15.

18: The method of claim 16, wherein said condition or disorder is one or more of type I diabetes, type II diabetes, obesity, appetite control, satiety, glucose intolerance, insulin resistance, metabolic syndrome, hyperlipidemia, hypercholesterolemia, atherosclerosis, inflammation, neurodegenerative diseases, alzheimers, stress disorders, and cerebrovascular conditions.

19: The method of claim 16, wherein said condition or disorder is one or more of type I diabetes, type II diabetes, glucose intolerance, insulin resistance, and metabolic syndrome.

20: The method of claim 16, wherein said administering is by a targeted drug delivery system.

21: The method of claim 20 wherein said targeted drug delivery system is a colon targeted drug delivery system.

22: The method of claim 3, wherein said condition or disorder is one or more of type I diabetes, type II diabetes, obesity, appetite control, satiety, glucose intolerance, insulin resistance, metabolic syndrome, hyperlipidemia, hypercholesterolemia, atherosclerosis, inflammation, neurodegenerative diseases, alzheimers, stress disorders, and cerebrovascular conditions.

23: The method of claim 3, wherein said condition or disorder is one or more of type I diabetes, type II diabetes, glucose intolerance, insulin resistance, and metabolic syndrome.

24: The method of claim 3, wherein said administering is by a targeted drug delivery system.

25: The method of claim 24 wherein said targeted drug delivery system is a colon targeted drug delivery system.

26: The method of claim 4, wherein said administering is by a targeted drug delivery system.

27: The method of claim 26 wherein said targeted drug delivery system is a colon targeted drug delivery system.

28: The method of claim 5, wherein said administering is by a targeted drug delivery system.

29: The method of claim 28 wherein said targeted drug delivery system is a colon targeted drug delivery system.

30: The method of claim 17, wherein said condition or disorder is one or more of type I diabetes, type II diabetes, obesity, appetite control, satiety, glucose intolerance, insulin resistance, metabolic syndrome, hyperlipidemia, hypercholesterolemia, atherosclerosis, inflammation, neurodegenerative diseases, alzheimers, stress disorders, and cerebrovascular conditions.

31: The method of claim 17, wherein said condition or disorder is one or more of type I diabetes, type II diabetes, glucose intolerance, insulin resistance, and metabolic syndrome.

32: The method of claim 17, wherein said administering is by a targeted drug delivery system.

33: The method of claim 32 wherein said targeted drug delivery system is a colon targeted drug delivery system.

34: The method of claim 18, wherein said administering is by a targeted drug delivery system.

35: The method of claim 34 wherein said targeted drug delivery system is a colon targeted drug delivery system.

36: The method of claim 18, wherein said administering is by a targeted drug delivery system.

37: The method of claim 36 wherein said targeted drug delivery system is a colon targeted drug delivery system.
